(19) United States
(12) Patent Application Publication
(10) Pub. No.: US 2024/0209005 A1
(43) Pub. Date: Jun. 27, 2024

(54) BICYCLIC HETEROARYL BORONATE DERIVATIVES AS ECTONUCLEOTIDE PYROPHOSPHATASE PHOSPHODIESTERASE 1 INHIBITORS

(71) Applicant: RIBOSCIENCE LLC, Palo Alto, CA (US)

(72) Inventor: Ronald HAWLEY, Palo Alto, CA (US)

(21) Appl. No.: 18/550,601

(22) PCT Filed: Mar. 15, 2022
(86) PCT No.: PCT/US22/20429

§ 371 Date: Sep. 14, 2023

Related U.S. Application Data
(60) Provisional application No. 63/161,652, filed on Mar. 16, 2021.

(51) Int. Cl. C07F 5/02; A61K 31/69; A61K 45/06

(52) U.S. Cl. CPC C07F 5/025; A61K 31/69; A61K 45/06

(57) ABSTRACT

The present disclosure provides certain bicyclic heteroaryl boronate compounds that inhibit ectonucleotide pyrophosphatase/phosphodiesterase 1 (ENPP1) enzymatic activity and are therefore useful for the treatment of diseases and conditions modulated at least in part by ENPP1. In some embodiments, the bicyclic heteroaryl compounds includes those of Formula (I). Also provided herein are pharmaceutical compositions containing such compounds and processes for preparing such compounds.

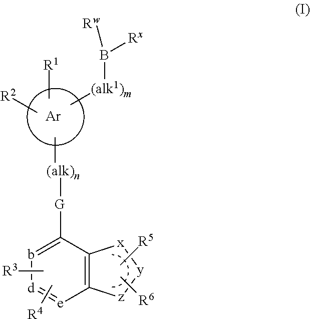

### CROSS-REFERENCES TO RELATED APPLICATIONS

This application claims the benefit of priority under 35 U.S.C. § 119(e) to U.S. Provisional Application Ser. No. 63/161,652 filed Mar. 16, 2021, the disclosure of which is incorporated herein by reference in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF THE DISCLOSURE

The present disclosure provides certain bicyclic heteroaryl boronate compounds that inhibit ectonucleotide pyrophosphatase/phosphodiesterase 1 (ENPP1) enzymatic activity and are therefore useful for the treatment of diseases treatable by inhibition of ENPP1. Also provided are pharmaceutical compositions containing such compounds and processes for preparing such compounds.

### BACKGROUND

ENPP1 enzyme is present in a wide range of tissues and cell types, such as lymphocytes, macrophages, liver, brain, heart, kidney, vascular smooth muscle cells, and chondrocytes. ENPP1 hydrolyzes ATP and other nucleoside triphosphates and releases AMP or other nucleoside monophosphates as well as pyrophosphate (PPi) (Kato K et al. 2012 PNAS 109:16876-16881; Hessle L et al. 2002 PNAS 99:9445-9449). The enzyme can also hydrolyze other nucleoside monophosphate esters (Kato K et al. 2012 PNAS 109:16876-16881). ENPP1 has been identified as the dominant 2′-3′-cGAMP hydrolase in cultured cells, tissue extracts and blood (Li L et al. 2014 Nat Chem Biol 10:1043-1048). Tissues and blood from ENPP1 knockout mice lack 2′-3′-cGAMP hydrolase activity. Elevated levels of ENPP1 have been associated with calcific aortic valve disease (CAVD) and calcium pyrophosphate dihydrate (CPPD) disease, an inflammatory disease resulting from CPPD crystal deposits in the joint and surrounding tissues (Cote N et al. 2012 Eur J Pharmacol 689:139-146; Johnson K et al. 2001 Arthritis Rheum 44:1071). ENPP1 expression is upregulated in certain hepatocellular carcinomas, glioblastomas, melanomas, testicular, pancreatic and thyroid and breast cancers and has been associated with resistance to chemotherapy (see Lau W M et al. 2013 PLoS One 8:5; Bageritz J et al. 2014 Mol Cell Oncology 1:3; Bageritz J et al. 2014 Cell Death, Differentiation 21:929-940; Umar A et al. 2009 Mol Cell Proteomics 8:1278-1294). ENPP1 upregulation and variants of ENPP1 are also associated with insulin resistance and type 2 diabetes (Meyre D et al. 2005 Nat Genet 37:863-867; Maddux B A et al. 1995 Nature 373:448-451; Rey D et al. 2012 Mol Biol Rep 39:7687-7693) and enzyme activity of ENPP1 was reported to be required for the inhibition of insulin receptor signaling (Chin C N et al. 2009 Eur J Pharmacol 606:17-24).

Cyclic GMP-AMP synthase (cGAS) is a pattern recognition receptor that synthesizes the endogenous messenger molecule cGAMP from ATP and GTP in response to the presence of DNA derived from viruses, bacteria, damaged mitochondria or cancer cells. The cGAMP molecule then binds to the stimulator of interferon genes (STING) protein, which initiates a signaling response that activates innate immunity and results in the production of type I interferon, antiviral and immune-stimulatory cytokines (Sun L et al. 2013 Science 339:786-791; Wu J et al. 2013 Science 339:826-830; Gao D et al. 2013 Science 341:903-906; Li X et al. 2013 Science 341:1390-1394; Schoggins J W et al. 2014 Nature 505:691-695; Wassermann R et al. 2015 Cell Host Microbe 17:799-810; Watson R O et al. 2015 Cell Host Microbe 17:811-819; Collins A et al. 2015 Cell Host Microbe 17:820-828; West A et al. 2015 Nature 520:533-557; Woo S R et al. 2014 Immunity 41:830-842; Deng L et al. 2014 Immunity 41:843-852; Chen Q et al. 2016 Nat Immunol 17:1142-1148). The cGAS enzyme, cGAMP messenger and STING are is also involved in host defense against RNA viruses and the immune control of tumor development (Aguirre S et al. 2012 PLoS Pathog 8: e1002934; Barber GN 2015 Nat Rev Immunol 15:760-770). ENPP1 has been identified as the enzyme that naturally hydrolyzes cGAMP and therefore counteracts the innate immune response against infectious agents, damaged cells and cancer cells (Li L et al. 2014 Nat Chem Biol 10:1043-1048). The efficacy of non-hydrolyzable cGAMP analogs in inducing functional immune responses is higher than that of natural, hydrolysable cGAMP (Li L et al. 2014 Nat Chem Biol 10:1043-1048; Corrales L et al. 2015 Cell Rep 11:1018-1030). Virus infection has been demonstrated to be facilitated by ENPP1 overexpression and is attenuated by silencing of ENPP1 (Wang J et al. 2018 Mol Immunol 95:56-63).

Inhibitors of cGAMP hydrolysis may therefore be used to increase the effectiveness of immune responses against cancer cells and tumors and against infections by RNA or DNA viruses or bacteria. Inhibitors of ENPP1 and of cGAMP or nucleoside triphosphate hydrolysis may also be used for the treatment of inflammatory diseases that are associated with elevated nucleotidase levels, reduced nucleoside triphosphate, reduced cGAMP or reduced nucleoside monophosphate ester levels or diseases associated with elevated nucleoside or nucleoside monophosphate levels. For these reasons, ENPP1 is an attractive therapeutic target for the treatment of diseases, including cancer.

The present disclosure addresses these needs and provides related advantages as well.

### SUMMARY

In a first aspect, provided is a compound of Formula (I):

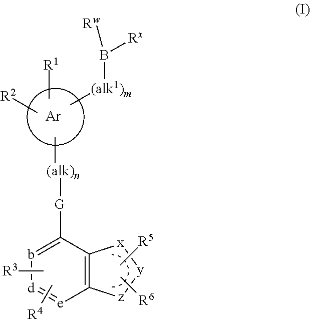

wherein:
   [symbol] dashed line is a bond between x and y or y and z;
   b, d, and e are CH; or one or two of b, d, and e are N and remaining of b, d, and e are CH;
   one of x and z is NH, O, or S and the other of x and z is CH or N; and y is CH or N; provided that, at least one of y and the x or z that is CH or N, is CH;
   G is a bond, NR (where R is hydrogen or alkyl), O, S, or SO₂;
   alk is alkylene optionally substituted with one, two, or three halo or alkynylene, provided that when alk is alkynylene G is a bond and n is 1;
   alk¹ is alkylene optionally substituted with one, two, or three halo;
   m and n are independently 0 or 1; provided that at least one of m and n is 1;
   Ar is aryl or heteroaryl;
   R^{w} and R^{x} are independently selected from hydroxy, alkoxy, —Oaryl (where aryl is optionally substituted with one to three substituents independently selected from alkyl, alkenyl, alkoxy, halo, haloalkyl, amino, alkylamino, dialkylamino, cyano, and nitro), —O—(CH₂)OCOR^{a}(where R^{a} is alkyl), —O-(alk²)OR (where alk² is alkylene and R^{b} is alkyl), —S—(CH₂)₂SCOR^{c} (where R^{c} is alkyl), or —NR^{c}—(CHR^{d})OCOR^{f} (where R^{d} is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl either alone or as part of phenylalkyl is optionally substituted with one to three substituents independently selected from alkyl, alkoxy, halo, hydroxy, cyano and nitro), R^{f} is alkyl or benzyl and R^{e} is hydrogen or R^{e} together with R^{d} forms —(CH₂)₃—); or
      R^{w} and R^{x} together with the boron atom to which they are attached can form a ring selected from:

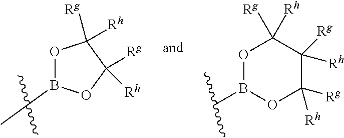

wherein each R^{g} and R^{h} is independently hydrogen or methyl;
R¹ and R² are independently absent, alkyl, alkoxy, halo, haloalkyl, haloalkoxy, or cyano;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl, either alone or part of heterocyclyloxy, and heterocyclylamino is optionally substituted with Rⁱ, R^{j}, or R^{k} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyl, heteroaryl, phenylalkenyl, heteroarylalkenyl, phenyloxy, or heteroaryloxy (where phenyl, by itself or as part of phenylalkenyl and phenyloxy and heteroaryl, by itself or as part of heteroarylalkenyl and heteroaryloxy, are optionally substituted with one, two, or three substituents where two of the optional substituents are independently selected from alkyl, hydroxy, alkoxy, halo, haloalkyl, haloalkoxy, and cyano); provided that (i) when two of b, d, and e are N, then at least one of R³ and R⁴ is absent;
R⁵ is absent, alkyl, hydroxy, alkoxy, alkoxycarbonyl, halo, acylamino, hydroxyalkylamino, alkoxyalkylamino, cyano, aminocarbonyl, alkylaminocarbonyl, dialkylaminocarbonyl, aminoalkyloxycarbonyl, alkoxyalkyloxycarbonyl, or hydroxyalkylaminocarbonyl; and
R⁶ is absent or alkyl; provided that one of R⁵ and R⁶ is absent when two of x, y, and z are other than CH; or
a pharmaceutically acceptable salt thereof,
provided that: (i) when b, e, x, and z are N, d and y are CH, G is S, n is 1, m is 0, R⁵ and R⁶ are absent, and one of R³ and R⁴ is absent, then the other of R³ and R⁴ is present; and (ii) the compound of Formula (I) is not (3-(((2-amino-7H-purin-6-yl)oxy)methyl)phenyl)boronic acid or 7-((4-(4,4,5,5-tetramethyl-1,3,2-dioxaborolan-2-yl)-1H-pyrazol-1-yl)methyl)-1H-indazole.

It is understood in Formula (I) and all of the subembodiments described herein that when one or more of R³, R⁴, R⁵, and R⁶ is present, these variable groups replace a hydrogen atom at the ring vertex to which each is attached. Additionally, in Formula (I) and all of the relevant subembodiments described herein, it is understood that the dashed line in the fused five membered ring comprising ring vertices x, y, and z indicates the presence of one double bond between either x and y or y and z.

In a second aspect, provided is a compound of Formula (IA):

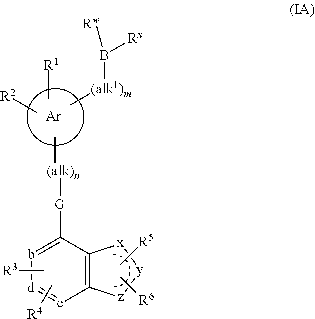

wherein:
   [symbol] dashed line is a bond between x and y or y and z;
   b, d, and e are CH; or one or two of b, d, and e are N and remaining of b, d, and e are CH;
   x is NH, O, or S;
   y and z are independently CH or N; provided that at least one of y and z is CH;
   G is NR (where R is hydrogen or alkyl), O, or S;
   alk is alkylene optionally substituted with one, two, or three halo;
   alk¹ is alkylene optionally substituted with one, two, or three halo;
   m and n are independently 0 or 1; provided that at least one of m and n is 1;
   Ar is aryl or heteroaryl;
   R^{w} and R^{x} are independently selected from hydroxy, alkoxy, —Oaryl (where aryl is optionally substituted with one to three substituents independently selected from alkyl, alkenyl, alkoxy, halo, haloalkyl, amino, alkylamino, dialkylamino, cyano, and nitro), —O—(CH₂)OCOR^{a}(where R^{a} is alkyl), —O-(alk²)OR (where alk² is alkylene and R^{b} is alkyl), —S—(CH₂)₂SCOR^{c} (where R^{c} is alkyl), or —NR^{e}—(CHR^{d})OCOR^{f} (where R^{d} is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl either alone or as part of phenylalkyl is optionally substituted with one to three substituents independently selected from alkyl, alkoxy, halo, hydroxy, cyano, and nitro), R^{f} is alkyl or benzyl and R^{e} is hydrogen or R^{e} together with R^{d} forms —(CH₂)₃—); or
   R^{w} and R^{x} together with the boron atom to which they are attached can form a ring selected from:

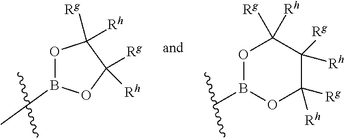

wherein each R^{g} and R^{h} is independently hydrogen or methyl;
R¹ and R² are independently absent, alkyl, alkoxy, halo, haloalkyl, haloalkoxy, or cyano;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with Rⁱ, R^{j}, or R^{k} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents where two of the optional substituents are independently selected from alkyl, hydroxy, alkoxy, halo, haloalkyl, haloalkoxy, and cyano); provided that (i) when two of b, d, and e are N, then one of R³ and R⁴ is absent and (ii) when G is S and one of R³ and R⁴ is hydrogen, then the other of R³ and R⁴ is other than hydrogen or amino;
R⁵ is absent, alkyl, hydroxy, halo, acylamino, hydroxyalkylamino, alkoxyalkylamino, aminocarbonyl, alkylaminocarbonyl, or dialkylaminocarbonyl; and
R⁶ is absent or alkyl; or
a pharmaceutically acceptable salt thereof.

In a third aspect, provided is a compound of Formula (IB):

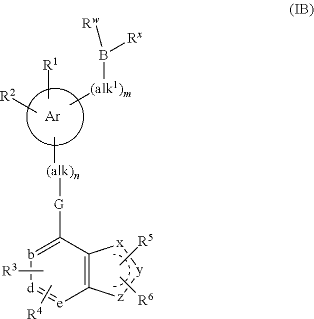

wherein:
   [symbol] dashed line is a bond between x and y or y and z;
   b, d, and e are CH; or one or two of b, d, and e are N and remaining of b, d, and e are CH;
   x is NH, O, or S;
   y and z are independently CH or N; provided that at least one of y and z is CH;
   G is NR (where R is hydrogen or alkyl) or 0;
   alk is alkylene optionally substituted with one, two, or three halo;
   alk¹ is alkylene optionally substituted with one, two, or three halo;
   m and n are independently 0 or 1; provided that at least one of m and n is 1;
   Ar is aryl or heteroaryl;
   R^{w} and R^{x} are independently selected from hydroxy, alkoxy, -Oaryl (where aryl is optionally substituted with one to three substituents independently selected from alkyl, alkenyl, alkoxy, halo, haloalkyl, amino, alkylamino, dialkylamino, cyano, and nitro), —O—(CH₂)OCOR^{a}(where R^{a} is alkyl), —O-(alk²)OR (where alk² is alkylene and R^{b} is alkyl), —S—(CH₂)₂SCOR^{c} (where R^{c} is alkyl), or —NR^{e}—(CHR^{d})OCOR^{f} (where R^{d} is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl either alone or as part of phenylalkyl is optionally substituted with one to three substituents independently selected from alkyl, alkoxy, halo, hydroxy, cyano, and nitro), R^{f} is alkyl or benzyl and R^{e} is hydrogen or R^{e} together with R^{d} forms —(CH₂)₃—); or
   R^{w} and R^{x} together with the boron atom to which they are attached can form a ring selected from:

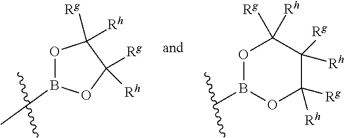

wherein each R^{g} and R^{h} is independently hydrogen or methyl;
R¹ and R² are independently absent, alkyl, alkoxy, halo, haloalkyl, haloalkoxy, or cyano;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with Rⁱ, R^{j}, or R^{k} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents where two of the optional substituents are independently selected from alkyl, hydroxy, alkoxy, halo, haloalkyl, haloalkoxy, and cyano);
R⁵ is absent, alkyl, hydroxy, halo, acylamino, hydroxyalkylamino, alkoxyalkylamino, aminocarbonyl, alkylaminocarbonyl, or dialkylaminocarbonyl; and
R⁶ is absent or alkyl; or
a pharmaceutically acceptable salt thereof.

In a fourth aspect, provided is a pharmaceutical composition comprising a compound of Formula (I), (IA), or (IB) (or any of the embodiments thereof described herein) or a pharmaceutically acceptable salt thereof and a pharmaceutically acceptable excipient.

In a fifth aspect, provided are methods of treating a disease or mediated by ENPP1 in a patient, preferably in a patient recognized as needing such a treatment, comprising administering to the patient (i) a compound of Formula (I), (IA), or (IB) (or any of the embodiments thereof described herein) or a pharmaceutically acceptable salt thereof in a therapeutically effective amount or (ii) a pharmaceutical composition comprising a compound of Formula (I), (IA), or (IB) (or any of the embodiments thereof described herein) or a pharmaceutically acceptable salt thereof and a pharmaceutically acceptable excipient in a therapeutically effective amount. In one embodiment, the disease is cancer such as hepatocellular carcinomas, glioblastomas, melanomas, testicular, pancreatic, thyroid and breast cancer. In another embodiment, the disease is an inflammatory disease e.g., calcific aortic valve disease and calcium pyrophosphate dihydrate. In yet another embodiment the disease metabolic disease e.g., type 2 diabetes or a viral infection.

In a sixth aspect, provided is a compound of Formula (I), (IA), or (IB) (or any embodiments thereof described herein) or a pharmaceutically acceptable salt thereof for use as a medicament. In one embodiment, the medicament is for use in the treatment of cancer such as hepatocellular carcinomas, glioblastomas, melanomas, testicular, pancreatic, thyroid and breast cancer. In another embodiment, the medicament is for use in the treatment of an inflammatory disease e.g., calcific aortic valve disease and calcium pyrophosphate dihydrate. In yet another embodiment, the medicament is for use in the treatment of a metabolic disease e.g., type 2 diabetes or a viral infection.

In a seventh aspect provided is a compound of Formula (I), (IA), or (IB) or a pharmaceutically acceptable salt thereof (and any embodiments thereof disclosed herein) for use in treating a disease in a patient in which the activity of ENPP1 contributes to the pathology and/or symptoms of the disease. In one embodiment, the disease is cancer such as hepatocellular carcinomas, glioblastomas, melanomas, testicular, pancreatic, thyroid and breast cancer. In another embodiment, the disease is an inflammatory disease e.g., calcific aortic valve disease and calcium pyrophosphate dihydrate. In yet another embodiment, the disease metabolic disease e.g., type 2 diabetes or a viral disease.

Unless noted otherwise, reference to an embodiment above includes subembodiments contained within such embodiments.

In any of the aforementioned aspects involving the treatment of cancer, are further embodiments comprising administering the compound of Formula (I), (IA), or (IB) or a pharmaceutically acceptable salt thereof (or any embodiments thereof disclosed herein) in combination with at least one additional anticancer. When combination therapy is used, the agents can be administered simultaneously or sequentially.

### DETAILED DESCRIPTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenyl” means a linear or branched monovalent hydrocarbon radical of two to six carbon atoms containing a double bond, e.g., ethenyl, propenyl, 2-propenyl, and the like.

“Alkenylene” means a linear or branched divalent hydrocarbon radical of two to six carbon atoms containing a double bond, e.g., ethenylene, propenylene, and the like.

“Alkynylene” means a linear or branched divalent hydrocarbon radical of two to six carbon atoms containing a tiple bond, e.g., ethynylene, propynylene, and the like.

“Alkylsulfonyl” means —SO₂R radical where R is alkyl as defined above, e.g., methylsulfonyl, ethylsulfonyl, and the like.

“Amino” means a —NH₂.

“Aminocarbonyl” means —CONH₂.

“Aminocarbonylmethyl” means —CH₂CONH₂.

“Aminocarbonylethyl” means —(CH₂)₂CONH₂.

“Alkylaminocarbonyl” means —CONHR radical where R is alkyl as defined above, e.g., methylaminocarbonyl, ethylaminocarbonyl, and the like.

“Acylamino” means —NHCOR radical where R is hydrogen, alkyl, phenyl, or heterocyclyl as defined above, e.g., formylamino, acetylamino, ethylcarbonylamino, benzoylamino, azetidin-1-ylcarbonylamino, and the like.

“Alkylamino” means a —NHR radical where R is alkyl as defined above, e.g., methylamino, ethylamino, propylamino, or 2-propylamino, and the like.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen or alkyl as defined above, e.g., aminomethyl, aminoethyl, methylaminomethyl, dimethylaminomethyl, and the like.

“Aminoalkylamino” means a —NR^{a}R^{b} radical where R^{a} is hydrogen or alkyl and R^{b} is aminoalkyl as defined above, e.g., aminoethylamino, dimethylaminoethylamino, diethylaminoethylamino, dimethylaminopropylamino, diethylaminopropylamino, and the like.

“Aminoalkyloxy” or “aminoalkoxy” means a —OR^{a} radical where R^{a} is aminoalkyl as defined above, e.g., aminoethyloxy, dimethylaminoethyloxy, diethylaminoethyloxy, dimethylaminopropyloxy, diethylaminopropyloxy, and the like.

“Aminoalkyloxycarbonyl” means a —COOR radical where R is aminoalkyl as defined above, e.g., aminoethyloxycarbonyl, dimethylaminomethyloxycarbonyl, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, such as one or two alkoxy groups, as defined above, e.g., 2-methoxyethyl, 1-, 2-, or 3-methoxypropyl, 2-ethoxyethyl, and the like.

“Alkoxyalkyloxycarbonyl” means a —COOR radical where R is alkoxyalkyl as defined above, e.g., methoxyethyloxycarbonyl, methoxymethyloxycarbonyl, and the like.

“Alkoxycarbonyl” means a —COOR radical where R is alkyl as defined above, e.g., methoxycarbonyl, ethoxycarbonyl, propoxycarbonyl, or 2-propoxycarbonyl, n-, iso-, or tert-butoxycarbonyl, and the like.

“Alkoxyalkylamino” means a —NRR′ radical where R is hydrogen or alkyl and R′ is alkoxyalkyl as defined above, e.g., methoxyethylamino, ethoxyethylamino, propoxypropylamino, ethoxypropylamino, and the like.

“Alkoxyalkyloxy” or “alkoxyalkoxy” means a —(O)R radical where R is alkoxyalkyl as defined above, e.g., methoxyethoxy, ethoxyethoxy, and the like.

“Amidinopropyl” refers to —(CH₂)₃NHC(═NH)NH₂ radical.

“Aryl” means a monovalent monocyclic or bicyclic aromatic hydrocarbon radical of 6 to ring atoms e.g., phenyl or naphthyl.

“Cycloalkyl” means a cyclic saturated monovalent hydrocarbon radical of three to ten carbon atoms, e.g., cyclopropyl, cyclobutyl, cyclopentyl, or cyclohexyl, and the like.

“Cycloalkyloxy” means a —OR radical where R is cycloalkyl (including specific cycloalkyl rings) as defined above e.g., cyclopropyloxy, and the like.

“Carboxy” means —COOH; “Carboxymethyl” means —CH₂COOH; and “Carboxyethyl” means —(CH₂)₂COOH.

“Dialkylaminocarbonyl” means —CONHRR′ where R and R′ are independently alkyl as defined above, e.g., dimethylaminocarbonyl, methylethylaminocarbonyl, and the like.

“Dialkylamino” means a —NRR′ radical where R and R′ are alkyl as defined above, e.g., dimethylamino, methylethylamino, and the like.

“Halo” means fluoro, chloro, bromo, or iodo, preferably fluoro or chloro.

“Haloalkyl” means alkyl radical as defined above, which is substituted with one or more halogen atoms, such as one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl. When hydroxyalkyl is —CH₂OH it is referred to herein as hydroxymethyl.

“Hydroxyalkylamino” means a —NR^{a}R^{b} radical where R^{a} is hydrogen or alkyl and R^{b} is hydroxyalkyl as defined above, e.g., hydroxyethylamino, hydroxypropylamino, and the like.

“Hydroxyalkylaminocarbonyl” means a —CONR^{a}R^{b} radical where R^{a} is hydrogen or alkyl and R^{b} is hydroxyalkyl as defined above, e.g., hydroxyethylaminocarbonyl, hydroxypropylaminocarbonyl, and the like.

“Hydroxyalkyloxy” or “hydroxyalkoxy” means a —OR^{a} radical where R^{a} is hydroxyalkyl as defined above, e.g., hydroxyethyloxy, hydroxypropyloxy, and the like.

“Heterocyclyl” means a saturated or unsaturated monovalent monocyclic group of 4 to 8 ring atoms in which one or two ring atoms are heteroatom selected from N, O, and S(O)ₙ, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, pyrrolidinyl, piperidinyl, homopiperidinyl, 2-oxopyrrolidinyl, 2-oxopiperidinyl, morpholinyl, piperazinyl, tetrahydro-pyranyl, thiomorpholinyl, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group.

“Heterocyclylalkyl” or “heterocycloalkyl” means a -(alkylene)-R radical where R is heterocyclyl ring (including specific heterocyclyl rings) as defined above e.g., tetraydrofuranylmethyl, piperazinylmethyl, morpholinylethyl, and the like.

“Heterocyclylamino” means a —NRR′ radical where R is hydrogen or alkyl and R′ is heterocyclyl (including specific heterocyclyl rings) as defined above.

“Heterocyclylalkylamino” or “heterocycloalkylamino” means a —NRR′ radical where R is hydrogen or alkyl and R′ is heterocyclylalkyl ring (including specific heterocyclyl rings) as defined above e.g., tetraydrofuranylmethylamino, piperazinylethylamino, morpholinylethylamino, piperidinylmethylamino, and the like.

“Heterocyclyloxy” means a —OR radical where R is heterocyclyl (including specific heterocyclyl rings) as defined above e.g., piperidinyloxy, pyrrolidinyloxy, and the like.

“Heterocyclylalkyloxy” or “heterocycloalkyloxy” means a —OR radical where R is heterocyclylalkyl ring (including specific heterocyclyl rings) as defined above e.g., tetraydrofuranylmethyloxy, piperazinylethyloxy, morpholinylethyloxy, piperidinylmethyloxy, and the like.

“Heteroaryl” means a monovalent monocyclic or fused bicyclic aromatic radical of 5 to ring atoms, unless otherwise stated, where one or more, (in one embodiment, one, two, or three), ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heteroaryloxy” means a —OR radical where R is heteroaryl (including specific heteroaryl rings) as defined above.

“Heteroarylalkenyl” means -(alkenylene)-R radical where R is heteroaryl and alkenylene are as defined above, e.g., 2-pyridinylethenylene, and the like.

“Methylthiomethyl” refers to —CH₂SCH₃ radical.

“Phenyloxy” means a —OR radical where R is phenyl.

“Phenylalkyl” means -(alkylene)-R radical where R is phenyl and alkylene is as defined above, e.g., benzyl, phenethyl, and the like.

“Phenylalkenyl” means -(alkenylene)-R radical where R is phenyl and alkenylene is as defined above, e.g., 2-phenylethenylene, and the like.

The present disclosure also includes protected derivatives of compounds of the present disclosure (I). For example, when compounds of the present disclosure contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of the present disclosure can be prepared by methods well known in the art.

The present disclosure also includes polymorphic forms and deuterated forms of the compound of the present disclosure and/or a pharmaceutically acceptable salt thereof.

A “pharmaceutically acceptable salt” of a compound means a salt that is pharmaceutically acceptable and that possesses the desired pharmacological activity of the parent compound. Such salts include: acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference in its entirety.

The compounds of the present disclosure may have asymmetric centers. Compounds of the present disclosure containing an asymmetrically substituted atom may be isolated in optically active or racemic forms. It is well known in the art how to prepare optically active forms, such as by resolution of materials. All chiral, diastereomeric, all mixtures of chiral or diasteromeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding (S) stereoisomer as an impurity i.e., the (S) stereoisomer in less than about 5%, preferably 2% by wt and then it is denoted as a mixture of R and S isomers, the amounts of R or S isomer in the mixture is greater than about 5%, preferably 2% w/w.

Certain compounds of the present disclosure can exist as tautomers and/or geometric isomers. All possible tautomers and cis and trans isomers, as individual forms and mixtures thereof are within the scope of this disclosure.

Additionally, as used herein the term alkyl includes all the possible isomeric forms of said alkyl group. Furthermore, when the cyclic groups such as aryl, heteroaryl, heterocyclyl are substituted, they include all the positional isomers. Furthermore, all hydrates of a compound of the present disclosure are within the scope of this disclosure.

Certain structures provided herein are drawn with one or more floating substituents e.g., in the structure:

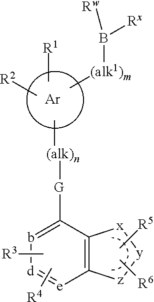

of Formula (I), (IA), and (IB), R¹, R², R³, R⁴, R⁵, and R⁶ are floating substituents. Unless provided otherwise or otherwise clear from the context, the floating substituent(s) may be present on any atom of the ring through which the substituent is drawn, where chemically feasible and valency rules permitting. For example, in the structure:

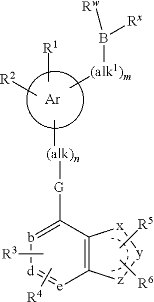

of Formula (I), (IB), and (IC), the R³ substituent can replace any hydrogen on the six membered aromatic ring portion of the bicyclic ring system when any of b, d, and e is CH and is not already substituted by the R⁴ substituent.

The compounds of the present disclosure may also contain unnatural amounts of isotopes at one or more of the atoms that constitute such compounds. Unnatural amounts of an isotope may be defined as ranging from the amount found in nature to an amount 100% of the atom in question. that differ only in the presence of one or more isotopically enriched atoms. Exemplary isotopes that can be incorporated into compounds of the present invention, such as a compound of Formula (I) (and any embodiemtn thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I, respectively. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds disclosed herein, including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

“Oxo” or “carbonyl” means ═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “patient” is generally synonymous with the term “subject” and includes all mammals including humans. Examples of patients include humans, livestock such as cows, goats, sheep, pigs, and rabbits, and companion animals such as dogs, cats, and horses. Preferably, the patient is a human.

The terms “inhibiting” and “reducing,” or any variation of these terms in relation of EPPI, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of EPP1 activity compared to normal.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

In one embodiment, treating or treatment includes inhibiting or relieving the disease.

A “therapeutically effective amount” means the amount of a compound of the present disclosure and/or a pharmaceutically acceptable salt thereof that, when administered to a patient for treating a disease, is sufficient to effect such treatment for the disease. The “therapeutically effective amount” will vary depending on the compound, the disease and its severity and the age, weight, etc., of the mammal to be treated.

“Thiomethyl” refers to —CH₂SH radical.

Representative compound of Formula (I) are disclosed in Table 1 below:

| Cpd # | Structure | Name |
|---|---|---|
| 1 | 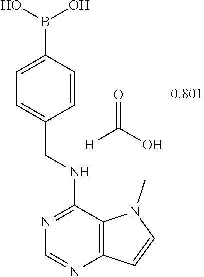 | 4-[([5-methylpyrrolo[3,2-d]pyrimidin-4- yl]amino)- methyl]phenylboronic acid; formic acid |
| 2 | 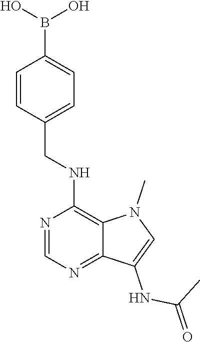 [SMILES: CC(=O)Nc1cn(C)c2c(NCc3ccc(B(O)O)cc3)ncnc12] | 4-[([7-acetamido-5-methylpyrrolo[3,2- d]pyrimidin-4-yl]- amino)methyl]phenylboronic acid |
| 3 | 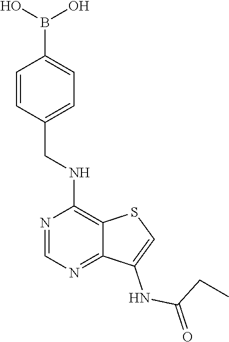 [SMILES: CCC(=O)Nc1csc2c(NCc3ccc(B(O)O)cc3)ncnc12] | 4-[([7-propanamidothieno[3,2-d]pyrimidin-4- yl]amino)-methyl]phenylboronic acid |
| 4 | 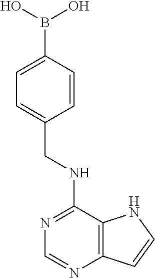 [SMILES: OB(O)c1ccc(CNc2ncnc3cc[nH]c23)cc1] | 4-([5H-pyrrolo[3,2-d]pyrimidin-4- ylamino]methyl)phenyl-boronic acid |
| 5 | 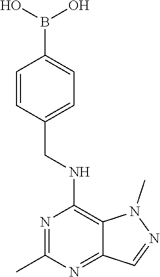 [SMILES: Cc1nc(NCc2ccc(B(O)O)cc2)c2c(cnn2C)n1] | 4-[([1,5-dimethylpyrazolo[4,3-d]pyrimidin-7- yl]amino)-methyl]phenylboronic acid |
| 6 | 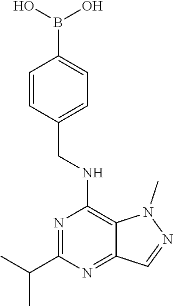 [SMILES: CC(C)c1nc(NCc2ccc(B(O)O)cc2)c2c(cnn2C)n1] | 4-[([5-isopropyl-1-methylpyrazolo[4,3- d]pyrimidin-7-yl]- amino)methyl]phenylboronic acid |
| 7 | 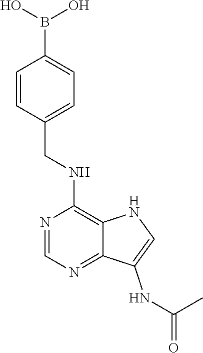 [SMILES: CC(=O)Nc1c[nH]c2c(NCc3ccc(B(O)O)cc3)ncnc12] | 4-[([7-acetamido-5H-pyrrolo[3,2- d]pyrimidin-4-yl]amino)- methyl]phenylboronic acid |
| 8 | 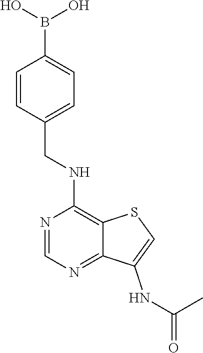 [SMILES: CC(=O)Nc1csc2c(NCc3ccc(B(O)O)cc3)ncnc12] | 4-[([7-acetamidothieno[3,2-d]pyrimidin-4- yl]amino)-methyl]phenylboronic acid |
| 9 | 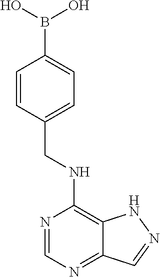 [SMILES: OB(O)c1ccc(CNc2ncnc3cn[nH]c23)cc1] | 4-([1H-pyrazolo[4,3-d]pyrimidin-7- ylamino]methyl)-phenylboronic acid |
| 10 | 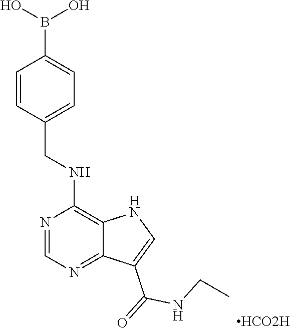 [SMILES: CCNC(=O)c1c[nH]c2c(NCc3ccc(B(O)O)cc3)ncnc12 ; "·HCO2H"] | 4-([[7-(ethylcarbamoyl)-5H-pyrrolo[3,2- d]pyrimidin-4-yl]- amino]methyl)phenylboronic acid; formic acid |
| 11 | 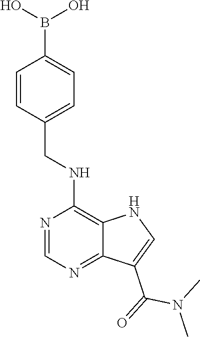 [SMILES: CN(C)C(=O)c1c[nH]c2c(NCc3ccc(B(O)O)cc3)ncnc12] | 4-([[7-(dimethylcarbamoyl)-5H-pyrrolo[3,2- d]pyrimidin-4-yl]- amino]methyl)phenylboronic acid |
| 12 | 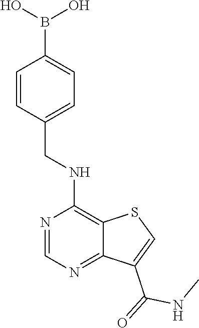 [SMILES: CNC(=O)c1csc2c(NCc3ccc(B(O)O)cc3)ncnc12] | 4-([[7-(methylcarbamoyl)thieno[3,2- d]pyrimidin-4-yl]- amino]methyl)phenylboronic acid |
| 13 | 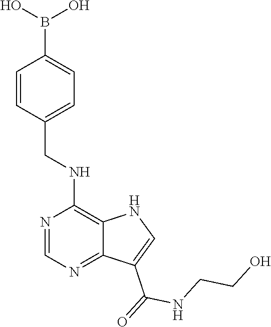 [SMILES: O=C(NCCO)c1c[nH]c2c(NCc3ccc(B(O)O)cc3)ncnc12] | 4-[([7-[(2-hydroxyethyl)carbamoyl]-5H- pyrrolo[3,2-d]-pyrimidin-4- yl]amino)methyl]phenylboronic acid |
| 14 | 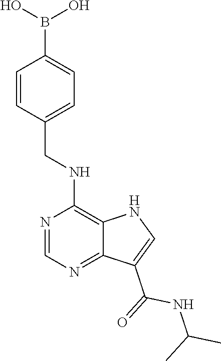 [SMILES: CC(C)NC(=O)c1c[nH]c2c(NCc3ccc(B(O)O)cc3)ncnc12] | 4-([[7-(isopropylcarbamoyl)-5H-pyrrolo[3,2- d]pyrimidin-4- yl]amino]methyl)phenylboronic acid |
| 15 | 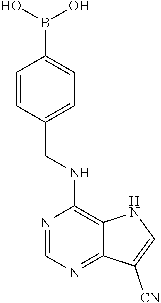 [SMILES: N#Cc1c[nH]c2c(NCc3ccc(B(O)O)cc3)ncnc12] | 4-[([7-cyano-5H-pyrrolo[3,2-d]pyrimidin-4- yl]amino)methyl]phenylboronic acid |
| 16 | 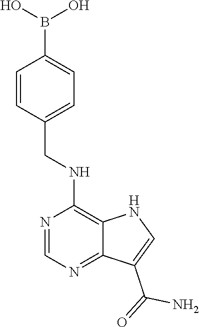 [SMILES: NC(=O)c1c[nH]c2c(NCc3ccc(B(O)O)cc3)ncnc12] | 4-[([7-carbamoyl-5H-pyrrolo[3,2- d]pyrimidin-4-yl]amino)- methyl]phenylboronic acid |
| 17 | 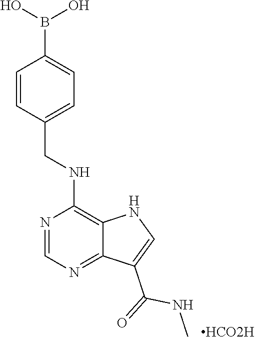 [SMILES: CNC(=O)c1c[nH]c2c(NCc3ccc(B(O)O)cc3)ncnc12 ; "·HCO2H"] | 4-([[7-(methylcarbamoyl)-5H-pyrrolo[3,2- d]pyrimidin-4- yl]amino]methyl)phenylboronic acid; formic acid |
| 18 | 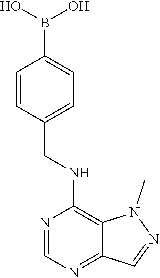 [SMILES: Cn1ncc2ncnc(NCc3ccc(B(O)O)cc3)c21] | 4-[([1-methylpyrazolo[4,3-d]pyrimidin-7- yl]amino)methyl]-phenylboronic acid |
| 19 | 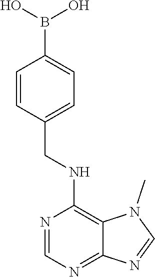 [SMILES: Cn1cnc2ncnc(NCc3ccc(B(O)O)cc3)c21] | 4-[[(7-methylpurin-6- yl)amino]methyl]phenylboronic acid |
| 20 | 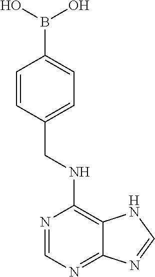 [SMILES: OB(O)c1ccc(CNc2ncnc3nc[nH]c23)cc1] | 4-[(7H-purin-6- ylamino)methyl]phenylboronic acid |
| 21 | 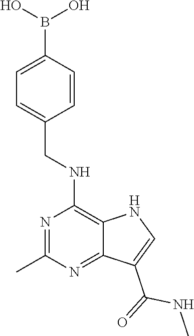 [SMILES: CNC(=O)c1c[nH]c2c(NCc3ccc(B(O)O)cc3)nc(C)nc12] | 4-([[2-methyl-7-(methylcarbamoyl)-5H- pyrrolo[3,2-d]-pyrimidin-4- yl]amino methyl)phenylboronic acid |
| 22 | 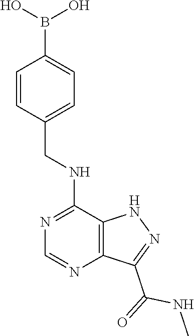 [SMILES: CNC(=O)c1n[nH]c2c(NCc3ccc(B(O)O)cc3)ncnc12] | 4-([3-(methylcarbamoyl)-1H-pyrazolo[4,3- d]pyrimidin-7- yl]amino]methyl)phenylboronic acid |
| 23 | 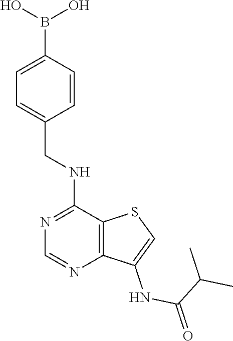 [SMILES: CC(C)C(=O)Nc1csc2c(NCc3ccc(B(O)O)cc3)ncnc12] | 4-([[7-(2-methylpropanamido)thieno[3,2- d]pyrimidin-4-yl]- amino]methyl)phenylboronic acid |
| 24 | 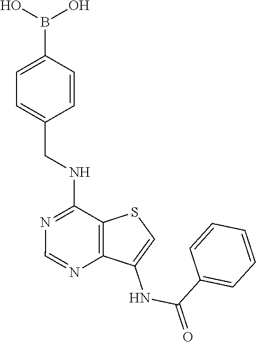 [SMILES: O=C(Nc1csc2c(NCc3ccc(B(O)O)cc3)ncnc12)c1ccccc1] | 4-[([7-benzamidothieno[3,2-d]pyrimidin-4- yl]amino)-methyl]phenylboronic acid |
| 25 | 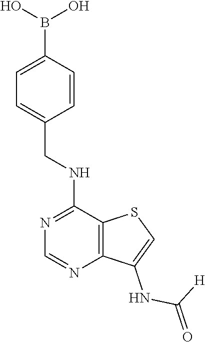 [SMILES: O=CNc1csc2c(NCc3ccc(B(O)O)cc3)ncnc12] | 4-[([7-formamidothieno[3,2-d]pyrimidin-4- yl]amino)-methyl]phenylboronic acid |
| 26 | 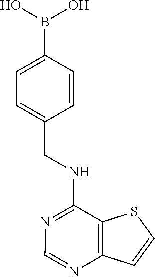 [SMILES: OB(O)c1ccc(CNc2ncnc3ccsc23)cc1] | 4-([thieno[3,2-d]pyrimidin-4- ylamino]methyl)phenyl-boronic acid. |
| 27 | 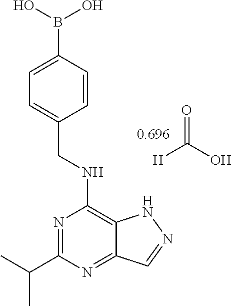 | 4-[([5-isopropyl-1H-pyrazolo[4,3- d]pyrimidin-7-yl]- amino)methyl]phenylboronic acid formic acid salt |
| 28 | 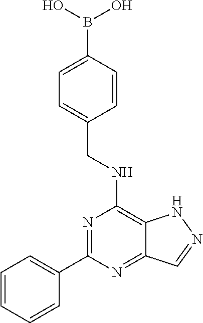 [SMILES: OB(O)c1ccc(CNc2nc(-c3ccccc3)nc3cn[nH]c23)cc1] | 4-[([5-phenyl-1H-pyrazolo[4,3-d]pyrimidin- 7-yl]amino)-methyl]phenylboronic acid |
| 29 | 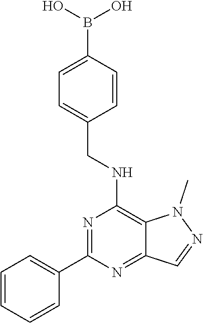 [SMILES: Cn1ncc2nc(-c3ccccc3)nc(NCc3ccc(B(O)O)cc3)c21] | 4-[([1-methyl-5-phenylpyrazolo[4,3- d]pyrimidin-7-yl]-amino)- methyl]phenylboronic acid |
| 30 | 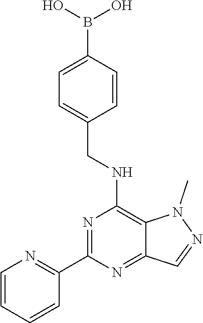 [SMILES: Cn1ncc2nc(-c3ccccn3)nc(NCc3ccc(B(O)O)cc3)c21] | 4-([[1-methyl-5-(pyridin-2-yl)pyrazolo[4,3- d]pyrimidin-7-yl]amino]- methyl)phenylboronic acid |
| 31 | 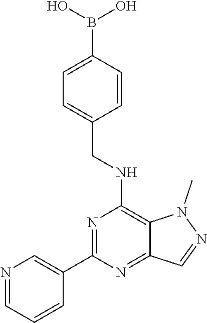 [SMILES: Cn1ncc2nc(-c3cccnc3)nc(NCc3ccc(B(O)O)cc3)c21] | 4-([[1-methyl-5-(pyridin-3-yl)pyrazolo[4,3- d]pyrimidin-7-yl]amino]methyl)- phenylboronic acid |
| 32 | 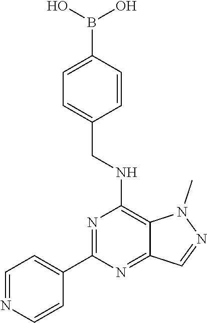 [SMILES: Cn1ncc2nc(-c3ccncc3)nc(NCc3ccc(B(O)O)cc3)c21] | 4-([[1-methyl-5-(pyridin-4-yl)pyrazolo[4,3- d]pyrimidin-7- yl]amino]methyl)phenylboronic acid |
| 33 | 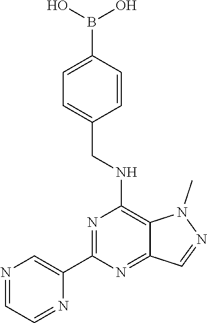 [SMILES: Cn1ncc2nc(-c3cnccn3)nc(NCc3ccc(B(O)O)cc3)c21] | 4-([[1-methyl-5-(pyrazin-2-yl)pyrazolo[4,3- d]pyrimidin-7-yl]amino]methyl)- phenylboronic acid |
| 34 | 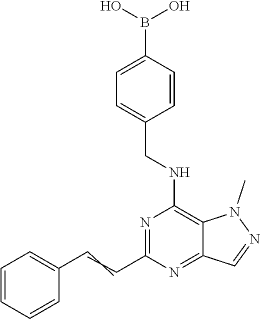 [SMILES: Cn1ncc2nc(C=Cc3ccccc3)nc(NCc3ccc(B(O)O)cc3)c21] | 4-[([1-methyl-5-[2- phenylethenyl]pyrazolo[4,3-d]pyrimidin-7- yl]amino)-methyl]phenylboronic acid |
| 35 | 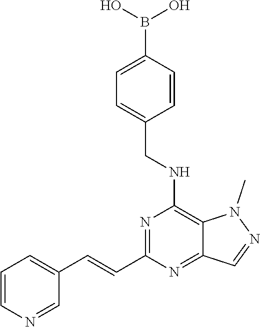 [SMILES: Cn1ncc2nc(/C=C/c3cccnc3)nc(NCc3ccc(B(O)O)cc3)c21] | 4-[([1-methyl-5-[(E)-2-(pyridin-3- yl)ethenyl]pyrazolo[4,3-d]pyrimidin-7- yl]amino)methyl]phenylboronic acid |
| 36 | 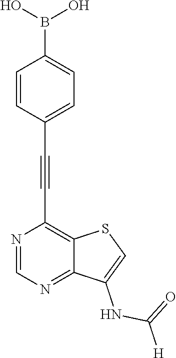 [SMILES: O=CNc1csc2c(C#Cc3ccc(B(O)O)cc3)ncnc12] | 4-(2-[7-formamidothieno[3,2-d]pyrimidin-4- yl]ethynyl)-phenylboronic acid |
| 37 | 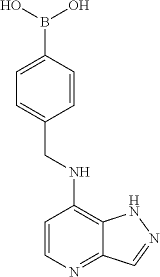 [SMILES: OB(O)c1ccc(CNc2ccnc3cn[nH]c23)cc1] | 4-([1H-pyrazolo[4,3-b]pyridin-7- ylamino]methyl)phenyl-boronic acid |
| 38 | 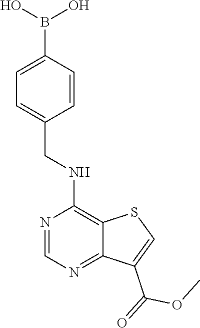 [SMILES: COC(=O)c1csc2c(NCc3ccc(B(O)O)cc3)ncnc12] | 4-([[7-(methoxycarbonyl)thieno[3,2- d]pyrimidin-4-yl]-amino]methyl)- phenylboronic acid |
| 39 | 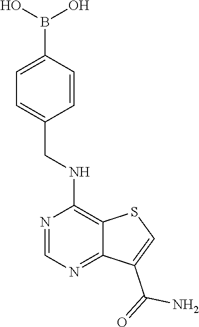 [SMILES: NC(=O)c1csc2c(NCc3ccc(B(O)O)cc3)ncnc12] | 4-[([7-carbamoylthieno[3,2-d]pyrimidin-4- yl]amino)-methyl]phenylboronic acid |
| 40 | 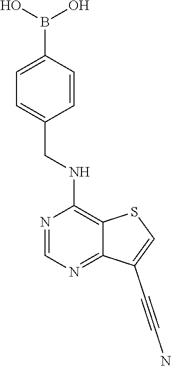 [SMILES: NC#Cc1csc2c(NCc3ccc(B(O)O)cc3)ncnc12] | 4-[([7-cyanothieno[3,2-d]pyrimidin-4- yl]amino)-methyl]phenylboronic acid |

### EMBODIMENTS

In embodiments A to R, A1 to R1, and subembodiments contained therein, the present disclosure includes:

### Embodiment A

In embodiment A, provided is a compound of Formula (I) or a pharmaceutically acceptable salt thereof, where:
alk is alkylene optionally substituted with one to three halo;
one or both of R³ and R⁴ are other than phenyl, heteroaryl, phenylalkenyl, and heteroarylalkenyl (where phenyl, by itself or as part of phenylalkenyl and heteroaryl, by itself or as part of heteroarylalkenyl are optionally substituted with one, two, or three substituents where two of the optional substituents are independently selected from alkyl, hydroxy, alkoxy, halo, haloalkyl, haloalkoxy, and cyano); and
R⁵ is absent, alkyl, hydroxy, halo, acylamino, hydroxyalkylamino, alkoxyalkylamino, cyano, aminocarbonyl, alkylaminocarbonyl, or dialkylaminocarbonyl;
and other groups are as defined in the Summary; or a compound of Formula (IA) or (IB), or a pharmaceutically acceptable salt thereof, as defined in the Summary above.

(Ai) In subembodiment (Ai) of embodiment A, the compounds, or a pharmaceutically acceptable salt thereof, have a structure of Formula (I) as defined in the Embodiment A.

(Aii) In subembodiment (Aii) of embodiment A, the compounds, or a pharmaceutically acceptable salt thereof, have a structure of Formula (IA) as defined in the Summary above.

(Aiii) In subembodiment (Aiii) of embodiment A, the compounds, or a pharmaceutically acceptable salt thereof, have a structure of Formula (IB) as defined in the Summary above.

### Embodiment B

In embodiment B, the compounds of any one of embodiment A and subembodiments contained therein (i.e., (Ai), (Aii) and (Aiii) and subembodiment contained in (Ai)), or a pharmaceutically acceptable salt thereof, have a structure of formula (Ia) or (Ib):

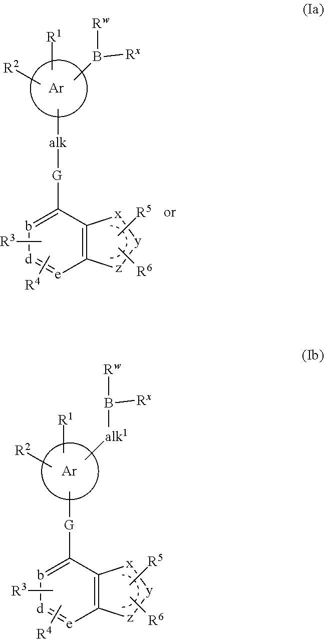

(Bi) In subembodiment (Bi) of embodiment B, the compounds of embodiment B, or a pharmaceutically acceptable salt thereof, have a structure of formula (Ia).

(Bii) In subembodiment (Bii) of embodiment B, the compounds of embodiment B, or a pharmaceutically acceptable salt thereof, have a structure of formula (Ib).

### Embodiment C

In embodiment C, the compounds of any one of embodiment A and subembodiments contained therein (i.e., (Ai), (Aii) and (Aiii) and subembodiment contained in (Ai)), or a pharmaceutically acceptable salt thereof, have a structure of formula (Ic) or (Id):

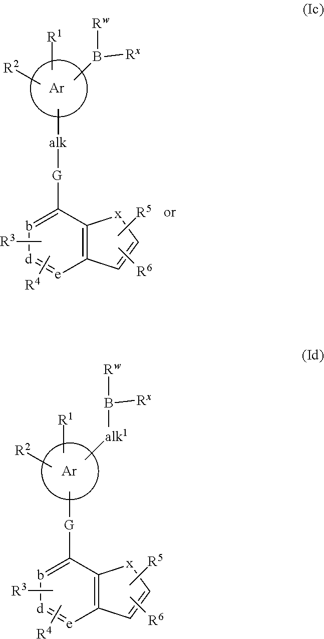

wherein x is NH, O, or S.

(Ci) In subembodiment (Ci) of embodiment C, the compounds of embodiment C, or a pharmaceutically acceptable salt thereof, have a structure of formula (Ic).

(Cii) In subembodiment (Cii) of embodiment C, the compounds of embodiment C, or a pharmaceutically acceptable salt thereof have a structure of formula (Id).

(Ciii) In subembodiment (Ciii) of embodiment C, the compounds of any one of embodiment C and subembodiments (Ci), and (Cii) contained therein, or a pharmaceutically acceptable salt thereof, are wherein x is NH.

(Civ) In subembodiment (Civ) of embodiment C, the compounds of any one of embodiment C and subembodiments (Ci), and (Cii) contained therein, or a pharmaceutically acceptable salt thereof, are wherein x is O.

(Cv) In subembodiment (Cv) of embodiment C, the compounds of any one of embodiment C and subembodiments (Ci), and (Cii) contained therein, or a pharmaceutically acceptable salt thereof, are wherein x is S.

(Cvi) In subembodiment (Cvi) of embodiment C, the compounds of any one of embodiment C and subembodiments (Ci) to (Ciii) contained therein, or a pharmaceutically acceptable salt thereof, are where, when x is NH, then R⁶ is either attached to the nitrogen of NH or the carbon of the 5-membering ring that is adjacent to x, and R⁵ is attached to carbon of the 5-membered ring that is adjacent to a bridgehead carbon, i.e.

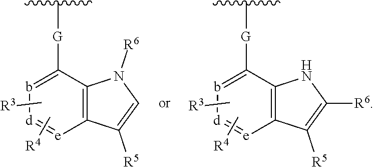

(Cvii) In subembodiment (Cvii) of embodiment C, the compounds of any one of embodiment C and subembodiments (Ci), (Cii), (Civ) and (Cv) contained therein, or a pharmaceutically acceptable salt thereof, are where, when, x is O or S, R⁶ is attached to the carbon of the 5-membered ring that is adjacent to x and R⁵ when present is attached to carbon of the 5-membered ring that is adjacent to a bridgehead carbon, i.e.

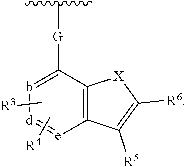

### Embodiment D

In embodiment D, the compound of any one of embodiment A and subembodiments contained therein (i.e., (Ai), (Aii), and (Aiii) and subembodiment contained in (Ai)), or a pharmaceutically acceptable salt thereof, has a structure of formula (Ie) or (If):

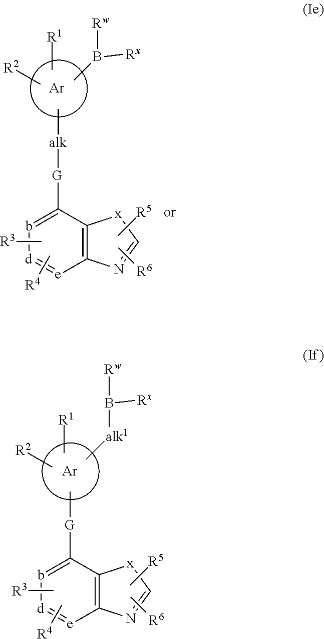

wherein x is NH, O, or S.

(Di) In subembodiment (Di) of embodiment D, the compounds of embodiment D, or a pharmaceutically acceptable salt thereof, have a structure of formula (Ie).

(Dii) In subembodiment (Dii) of embodiment D, the compounds of embodiment D, or a pharmaceutically acceptable salt thereof, have a structure of formula (If).

(Diii) In subembodiment (Diii) of embodiment D, the compounds of any one of embodiment D and subembodiments (Di), and (Dii) contained therein, or a pharmaceutically acceptable salt thereof, are wherein x is NH.

(Div) In subembodiment (Div) of embodiment D, the compounds of any one of embodiment D and subembodiments (Di), and (Dii) contained therein, or a pharmaceutically acceptable salt thereof, are wherein x is O.

(Dv) In subembodiment (Dv) of embodiment D, the compounds of any one of embodiment D and subembodiments (Di), and (Dii) contained therein, or a pharmaceutically acceptable salt thereof, are wherein x is S.

(Dvi) In subembodiment (Dvi) of embodiment D, the compounds of any one of embodiment D and subembodiments (Di) to (Diii) contained therein, or a pharmaceutically acceptable salt thereof, are where, when x is NH, then R⁶ is attached to the nitrogen of NH and R⁵ is attached to carbon of the 5-membered ring that is adjacent to x, i.e.

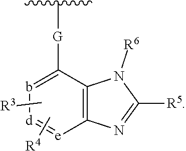

(Dvii) In subembodiment (Dvii) of embodiment D, the compounds of any one of embodiment D and subembodiments (Di), (Dii), (Div) and (Dv) contained therein, or a pharmaceutically acceptable salt thereof, are where, when, x is O or S, R⁶ is absent and R⁵ is attached to carbon of the 5-membered ring that is adjacent to x, i.e.

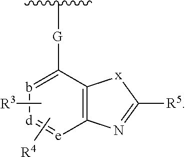

### Embodiment E

In embodiment E, the compounds of any one of embodiment A and subembodiments contained therein (i.e., (Ai), (Aii), and (Aiii) and subembodiment contained in (Ai)), or a pharmaceutically acceptable salt thereof, have a structure of formula (Ig) or (Ih):

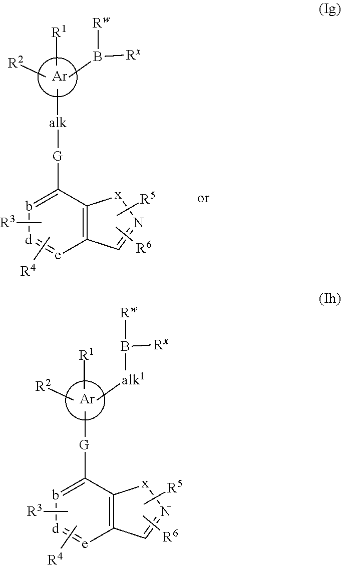

wherein x is NH, O, or S.

(Ei) In subembodiment (Ei) of embodiment E, the compounds of embodiment E, or a pharmaceutically acceptable salt thereof, have a structure of formula (Ig).

(Eii) In subembodiment (Eii) of embodiment E, the compounds of embodiment E, or a pharmaceutically acceptable salt thereof, have as structure of formula (Ih).

(Eiii) In subembodiment (Eiii) of embodiment E, the compounds of any one of embodiment E and subembodiments (Ei), and (Eii) contained therein, or a pharmaceutically acceptable salt thereof, are wherein x is NH.

(Eiv) In subembodiment (Eiv) of embodiment E, the compounds of any one of embodiment E and subembodiments (Ei) and (Eii) contained therein, or a pharmaceutically acceptable salt thereof, are wherein x is O.

(Ev) In subembodiment (Ev) of embodiment E, the compounds of any one of embodiment E and subembodiments (Ei) and (Eii) contained therein, or a pharmaceutically acceptable salt thereof, are wherein x is S.

(Evi) In subembodiment (Evi) of embodiment E, the compounds of any one of embodiment E and subembodiments (Ei) to (Eiii) contained therein, or a pharmaceutically acceptable salt thereof, are where, when x is NH, then R⁶, when present, is attached to the nitrogen of NH and R⁵ is attached to carbon of the 5-membered ring that is adjacent to a bridgehead carbon, i.e.

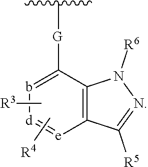

(Evii) In subembodiment (Evii) of embodiment E, the compounds of any one of embodiment E and subembodiments (Ei), (Eii), (Eiv) and (Ev) contained therein, or a pharmaceutically acceptable salt thereof, are where, when, x is O or S, R⁶ is absent and R⁵ is attached to carbon of the 5-membered ring that is adjacent to a bridgehead carbon, i.e.

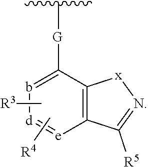

### Embodiment E1

In embodiment E1, the compounds of any one of embodiments A, B, C, D, and E and subembodiments contained therein, or a pharmaceutically acceptable salt thereof, are wherein G is NR, preferably NH.

### Embodiment E2

In embodiment E2, the compounds of any one of embodiments A, B, C, D, and E and subembodiments contained therein, or a pharmaceutically acceptable salt thereof, are wherein G is O.

### Embodiment E3

In embodiment E3, the compounds of any one of embodiments A, B, C, D, and E and subembodiments contained therein, or a pharmaceutically acceptable salt thereof, are wherein G is bond.

### Embodiment F

In embodiment F, the compounds of any one of embodiments A, B, C, D, E, E1, E2, and E3 and subembodiments contained therein, or a pharmaceutically acceptable salt thereof, are wherein b, d, and e are CH or C when attached to any one of R³ and R⁴.

### Embodiment G

In embodiment G, the compounds of any one of embodiments A, B, C, D, E, E1, E2, and E3 and subembodiments contained therein, or a pharmaceutically acceptable salt thereof, are wherein b is N and d, and e are CH or C when attached to any one of R³ and R⁴.

### Embodiment H

In embodiment H, the compounds of any one of embodiments A, B, C, D, E, E1 E2, and E3 and subembodiments contained therein, or a pharmaceutically acceptable salt thereof, are wherein d is N and b and e are CH or C when attached to any one of R³ and R⁴.

### Embodiment I

In embodiment I, the compounds of any one of embodiments A, B, C, D, E, E1, E2, and E3 and subembodiments contained therein, or a pharmaceutically acceptable salt thereof, are wherein e is N and b and d are CH or C when attached to any one of R³ and R⁴.

### Embodiment J

In embodiment J, the compounds of any one of embodiments A, B, C, D, E, E1, E2, and E3 and subembodiments contained therein, or a pharmaceutically acceptable salt thereof, are wherein b and e are N and d is CH or C when attached to any one of R³ and R⁴.

### Embodiment K

In embodiment K, the compounds of any one of embodiments A, B, C, D, E, E1, E2, E3, F, G, H, I, and J and subembodiment contained therein, or a pharmaceutically acceptable salt thereof, are wherein —B(R^{x})(R^{w}) is —B(OH)₂.

### Embodiment L

In embodiment L, the compounds of any one of embodiments A, B, C, D, E, E1, E2, E3, F, G, H, I, and J and subembodiment contained therein, or a pharmaceutically acceptable salt thereof, are wherein R^{w} and R^{x} are independently selected from hydroxy, alkoxy, —Oaryl (where aryl is optionally substituted with one to three substituents independently selected from alkyl, halo, haloalkyl, cyano, and nitro), —O—(CH₂)OCOR^{a} (where R^{a} is alkyl), or —O-(alk²)OR (where alk² is alkylene and R^{b} is alkyl). Preferably, R^{w} and R^{x} are independently selected from alkoxy, —Oaryl (where aryl is optionally substituted with one to three substituents independently selected from alkyl, halo, haloalkyl, cyano, and nitro), —O—(CH₂)OCOR^{a} (where R^{a} is alkyl), and —O-(alk²)OR (where alk² is alkylene and R^{b} is alkyl, such as methyl, isopropyl, n-propyl, isobutyl, or n-butyl). Preferably, R^{w} and R^{x} are independently hydroxy, alkoxy, or —Ophenyl (where phenyl is optionally substituted with one to three substituents independently selected from alkoxy, halo, haloalkyl, cyano, and nitro); or R^{w} and R^{x} together with the boron atom to which they are attached form a ring of formula (a) or (b):

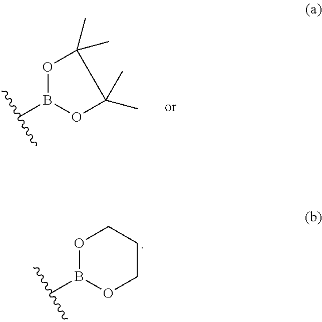

(Li) In subembodiment (Li) of embodiment L, the compounds of embodiment L, or a pharmaceutically acceptable salt thereof, are wherein R^{w} and R^{x} are independently selected from hydroxy and alkoxy.

(Lii) In subembodiment (Lii) of embodiment L, the compounds of embodiment L, or a pharmaceutically acceptable salt thereof, are wherein R^{w} and R^{x} together with the boron atom to which they are attached form a ring of formula (a) or (b):

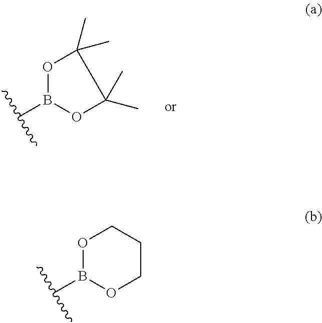

### Embodiment M

In embodiment M, the compounds of any one of embodiments A, B, C, D, E, E1, E2, E3, F, G, H, I, J, K, and L, and subembodiments contained therein or a pharmaceutically acceptable salt thereof, are wherein Ar is aryl or heteroaryl.

(Mi). In subembodiment (Mi) of embodiment M, the compounds of embodiment M, or a pharmaceutically acceptable salt thereof, are wherein Ar is phenyl.

(Mii). In subembodiment (Mii) of embodiment M, the compounds of embodiment M, or a pharmaceutically acceptable salt thereof, are wherein Ar is phenyl and —B(R^{w})(R^{x}) is attached to carbon of the phenyl ring that is meta to the carbon attaching the phenyl ring to remaining compound of Formula (I), (IA), (IB), and (Ia) to (Ih).

(Miii). In subembodiment (Miii) of embodiment M, the compounds of embodiment M, or a pharmaceutically acceptable salt thereof, are wherein Ar is phenyl and —B(R^{w})(R^{x}) is attached to carbon on the phenyl ring that is para to the carbon attaching the phenyl ring to remaining compound of Formula (I), (IA), (IB), and (Ia) to (Ih), respectively.

(Miv). In subembodiment (Miv) of embodiment M, the compounds of embodiment M, or a pharmaceutically acceptable salt thereof, are wherein Ar is heteroaryl.

(Mv). In subembodiment (Mv) of embodiment M, the compounds of embodiment M, or a pharmaceutically acceptable salt thereof, are wherein Ar is pyridinyl, pyrimidinyl, pyridazinyl, thienyl, furanyl, thiazolyl, oxazolyl, isoxazolyl, pyrazolyl, triazolyl, oxadiazolyl, or imidazolyl.

(Mvi). In subembodiment (Mvi) of embodiment M, the compounds of embodiment M, or a pharmaceutically acceptable salt thereof, are wherein Ar is a six-membered ring such as pyridinyl, pyrimidinyl, or pyridazinyl wherein Q is attached to carbon on the pyridinyl, pyrimidinyl, or pyridazinyl ring that is meta to the carbon attaching the pyridinyl, pyrimidinyl, or pyridazinyl ring to remaining compound of Formula (I), (IA), (IB), and (Ia) to (Ih), respectively.

(Mvii). In subembodiment (Mvii) of embodiment M, the compounds of embodiment M, or a pharmaceutically acceptable salt thereof, are those wherein Ar is benzofuranyl, quinolinyl, quinazolinyl, benzimidazolyl, indazolyl, benzotriazolyl, or benzoxazolyl.

### Embodiment N

In embodiment N, the compounds of any one of embodiments A, B, C, D, E, E1, E2, E3, F, G, H, I, J, K, L, and M, and subembodiments contained therein, or a pharmaceutically acceptable salt thereof, are wherein alk and alkⁱ are independently methylene, ethylene, or propylene.

(Ni) In subembodiment (Ni) of embodiment N, the compounds of embodiment N, or a pharmaceutically acceptable salt thereof, are those wherein alk and alkⁱ are methylene.

### Embodiment O

In embodiment O, the compounds of any one of embodiments A, B, D, E, E1, E2, E3, F, G, H, J, K, L, M, and N and subembodiments contained therein, or a pharmaceutically acceptable thereof, are wherein R⁶ is absent, methyl, or isopropyl.

(Oi). In subembodiment (Oi) of embodiment O, the compounds of embodiment O, or a pharmaceutically acceptable salt thereof, are those wherein R⁶ is absent.

(Oii). In subembodiment (Oii) of embodiment O, the compounds of embodiment O, or a pharmaceutically acceptable salt thereof, are wherein R⁶ is methyl or isopropyl.

### Embodiment P

In embodiment P, the compounds of any one of embodiments A, B, D, E, E1, E2, E3, F, G, H, J, K, L, M, N, and O, and subembodiments contained therein, or a pharmaceutically acceptable thereof, are wherein R⁵ is absent, alkyl, hydroxy, halo, acylamino, alkoxyalkylamino, cyano, aminocarbonyl, alkylaminocarbonyl, or dialkylaminocarbonyl.

(Pi). In subembodiment (Pi) of embodiment P, the compounds of embodiment P, or a pharmaceutically acceptable salt thereof, are wherein R⁵ is absent or alkyl.

(Pii). In subembodiment (Pii) of embodiment P, the compounds of embodiment P, or a pharmaceutically acceptable salt thereof, are wherein R⁵ is cyano, acylamino, aminocarbonyl, alkylaminocarbonyl, or dialkylaminocarbonyl. Preferably R⁵ is acylamino, aminocarbonyl, methylaminocarbonyl, ethylaminocarbonyl, dimethylaminocarbonyl, or isopropylcarbonyl.

### Embodiment Q

In embodiment Q, the compounds of any one of embodiments A, B, C, D, E, E1, E2, E3, F, G, H, I, J, K, L, M, N, O, and P, and subembodiments contained therein, or a pharmaceutically acceptable thereof, are wherein R¹ and R² are independently absent, methyl, ethyl, methoxy, fluoro, trifluoromethyl, trifluoromethoxy, or cyano.

(Qi). In subembodiment (Qi) of embodiment, Q, the compounds of embodiment Q, or a pharmaceutically acceptable salt thereof, are wherein R¹ and R² are absent.

### Embodiment R

(Ri) In embodiment Ri, the compounds of any one of embodiments A, B, C, D, E, E1, E2, E3, F, G, H, I, J, K, L, M, N, O, P, an Q and subembodiments contained therein, or a pharmaceutically acceptable thereof, are wherein R³ and R⁴ are independently absent, alkyl, alkoxy, hydroxy, amino, halo, haloalkyl, or haloalkoxy.

(Ri1). In subembodiment (Ri1) of embodiment (Ri), the compounds of embodiment (Ri), or a pharmaceutically acceptable thereof, are wherein R³ and R⁴ are independently absent, alkoxy, amino, or hydroxy.

(Ri2). In subembodiment (Ri2) of embodiment (Ri), the compounds of embodiment (Ri), or pharmaceutically acceptable thereof, are wherein R³ and R⁴ are independently alkoxy such as methoxy, ethoxy, or propoxy and are attached to to the six membered ring comprising b, d, and e of Formula (I), (IA), (IB). and (Ia) to (Ih) as shown below

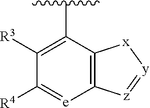

wherein the wavy line denotes the attachment point to the remainder of the molecule.

(Ri3). In subembodiment (Ri3) of embodiment (Ri), the compounds of embodiment (Ri), or pharmaceutically acceptable thereof, are wherein R³ and R⁴ are absent.

(Ri4). In subembodiment (Ri4) of embodiment (Ri), the compounds of embodiment (Ri), or pharmaceutically acceptable thereof, are those wherein when R⁴ is present, R⁴ is attached to the six membered ring comprising b, d, and e of Formula (I), (IA), (IB), and (Ia) to (Ih) as shown below:

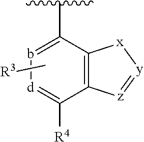

wherein the wavy line denotes the attachment point to the remainder of the molecule.

(Rii). In embodiment (Rii), the compounds of any one of embodiments A, B, C, D, E, E1, E2, E3, F, G, H, I, J, K, L, M, N, O, P, an Q and subembodiments contained therein, or a pharmaceutically acceptable thereof, are wherein:
R³ is absent, alkyl, alkoxy, hydroxy, halo, haloalkyl, or haloalkoxy; and
R⁴ is hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents, preferably one or two optional substituents, where two of the optional substituents are independently selected from alkyl, hydroxy, alkoxy, halo, haloalkyl, haloalkoxy, and cyano).

In a first subembodiment of embodiment (Rii), the compounds of embodiment (Rii), or pharmaceutically acceptable thereof, are wherein R³ is absent, methoxy, ethoxy, or hydroxy, preferably R³ is methoxy or ethoxy; and R⁴ is 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, or methoxy). Preferably, when R³ and R⁴ are present, R³ and R⁴ are attached to the six membered ring comprising b, d, and e of Formula (I), (A), (IB), and (Ia) to (Ih) as shown below

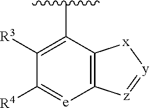

wherein the wavy line denotes the attachment point to the remainder of the molecule.

(Riii). In embodiment (Riii), the compounds of any one of embodiments A, B, C, D, E, E1, E2, E3, F, G, H, I, J, K, L, M, N, O, P, an Q and subembodiments contained therein, or a pharmaceutically acceptable thereof, are wherein R³ and R⁴ are independently hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl of phenyloxy and heteroaryl of heteroaryloxy are optionally substituted with one, two, or three substituents, preferably one or two optional substituents, where two of the optional substituents are independently selected from alkyl, hydroxy, alkoxy, halo, haloalkyl, haloalkoxy, and cyano).

In a first subembodiment of embodiment (Riii), the compounds of embodiment (Riii), or pharmaceutically acceptable thereof, are wherein R³ and R⁴ are independently 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, or methoxy). Preferably, R³ and R⁴ are attached to the six membered ring comprising b, d, and e of Formula (I), (IA), (IB), and (Ia) to (Ih) as shown below:

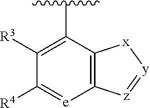

wherein the wavy line denotes the attachment point to the remainder of the molecule.

Further embodiments of the present disclosure include:

### Embodiment A1

In embodiment A1, provided is a compound of Formula (I) or a pharmaceutically acceptable salt thereof, where alk is alkynylene; and other groups are as defined in the Summary.

### Embodiment B1

In embodiment B1, the compounds of embodiment A1, or a pharmaceutically acceptable salt thereof, have a structure of formula (Ia1):

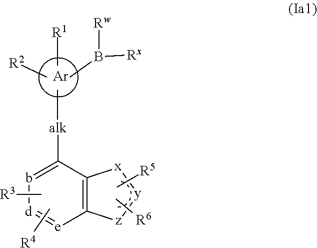

where x is N, O, or S.

### Embodiment C1

In embodiment C1, the compounds of embodiment A1 or B1, or a pharmaceutically acceptable salt thereof, have a structure of formula (Ic1):

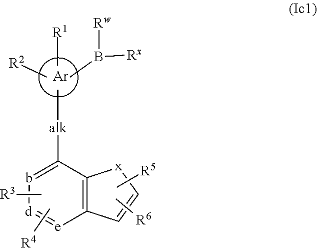

wherein x is NH, O, or S.

(C1i) In subembodiment (C1i) of embodiment C1, the compounds of embodiment C1, or a pharmaceutically acceptable salt thereof, are wherein x is NH.

(C1ii) In subembodiment (C1ii) of embodiment C1, or a pharmaceutically acceptable salt thereof, are wherein x is O.

(C1iii) In subembodiment (C1iii) of embodiment C1, or a pharmaceutically acceptable salt thereof, are wherein x is S.

(C1iv) In subembodiment (C1iv) of embodiment C1, the compounds of any one of embodiment C1 and subembodiment (C1i) contained therein, or a pharmaceutically acceptable salt thereof, are where, when x is NH, then R⁶ is either attached to the nitrogen of NH or the carbon of the 5-membering ring that is adjacent to x, and R⁵ is attached to carbon of the 5-membered ring that is adjacent to a bridgehead carbon, i.e.

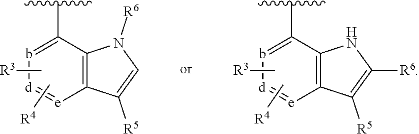

(C1v) In subembodiment (C1v) of embodiment C1, the compounds of any one of embodiment C1 and subembodiment ((C1ii) and (C1iii) contained therein, or a pharmaceutically acceptable salt thereof, are where, when, x is O or S, R⁶ is attached to the carbon of the 5-membered ring that is adjacent to x and R⁵ when present is attached to carbon of the 5-membered ring that is adjacent to a bridgehead carbon, i.e.

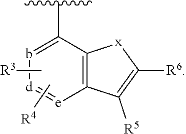

### Embodiment D1

In embodiment D1, the compound of embodiment A1 or Bi, or a pharmaceutically acceptable salt thereof, has a structure of formula (Ie1):

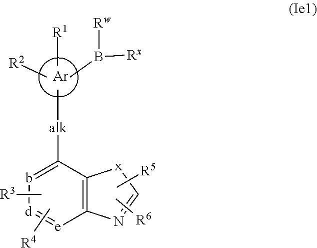

wherein x is NH, O, or S.

(D1i) In subembodiment (D1i) of embodiment D1, the compounds of embodiment D1, or a pharmaceutically acceptable salt thereof, are wherein x is NH.

(D1ii) In subembodiment (D1ii) of embodiment D1, the compounds of embodiment D1, or a pharmaceutically acceptable salt thereof, are wherein x is O.

(D1iii) In subembodiment (D1iii) of embodiment D1, the compounds of embodiment D1, or a pharmaceutically acceptable salt thereof, are wherein x is S.

(D1iv) In subembodiment (D1iv) of embodiment D1, the compounds of any one of embodiment D1 and subembodiment (D1i), or a pharmaceutically acceptable salt thereof, are where, when x is NH, then R⁶ is attached to the nitrogen of NH and R⁵ is attached to carbon of the 5-membered ring that is adjacent to x, i.e.

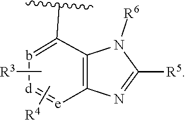

(D1v) In subembodiment (D1v) of embodiment D1, the compounds of any one of embodiment D1 and subembodiments (D1ii) and (D1iii), or a pharmaceutically acceptable salt thereof, are where, when, x is O or S, R⁶ is absent and R⁵ is attached to carbon of the 5-membered ring that is adjacent to x, i.e.

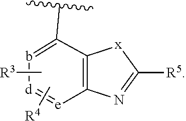

### Embodiment E1

In embodiment E1, the compounds of embodiment A1 or B1, or a pharmaceutically acceptable salt thereof, have a structure of formula (Ig1):

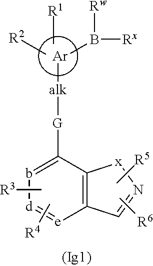

wherein x is NH, O, or S.

(E1i) In subembodiment (E1i) of embodiment E1, the compounds of embodiment E1, or a pharmaceutically acceptable salt thereof, are wherein x is NH.

(E1ii) In subembodiment (E1ii) of embodiment E1, the compounds of embodiment E1, or a pharmaceutically acceptable salt thereof, are wherein x is O.

(E1iii) In subembodiment (E1iii) of embodiment E1, the compounds of embodiment E, or a pharmaceutically acceptable salt thereof, are wherein x is S.

(E1iv) In subembodiment (E1iv) of embodiment E1, the compounds of any one of embodiment E1 and subembodiment (E1i) contained therein, or a pharmaceutically acceptable salt thereof, are where, when x is NH, then R⁶, when present, is attached to the nitrogen of NH and R⁵ is attached to carbon of the 5-membered ring that is adjacent to a bridgehead carbon, i.e.

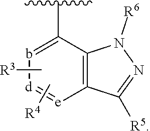

(E1v) In subembodiment (E1v) of embodiment E1, the compounds of any one of embodiment E1 and subembodiments (E1ii), and (E1iii) contained therein, or a pharmaceutically acceptable salt thereof, are where, when, x is O or S, R⁶ is absent and R⁵ is attached to carbon of the 5-membered ring that is adjacent to a bridgehead carbon, i.e.

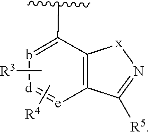

### Embodiment F1

In embodiment F1, the compounds of any one of embodiments A1, B1, C1, D1, and E1 and subembodiments contained therein, or a pharmaceutically acceptable salt thereof, are wherein b, d, and e are CH or C when attached to any one of R³ and R⁴.

### Embodiment G1

In embodiment G1, the compounds of any one of embodiments A1, B1, C1, D1, and E1 and subembodiments contained therein, or a pharmaceutically acceptable salt thereof, are wherein b is N and d, and e are CH or C when attached to any one of R³ and R⁴.

### Embodiment H1

In embodiment H1, the compounds of any one of embodiments A1, B1, C1, D1, and E1 and subembodiments contained therein, or a pharmaceutically acceptable salt thereof, are wherein d is N and b and e are CH or C when attached to any one of R³ and R⁴.

### Embodiment I1

In embodiment I1, the compounds of any one of embodiments A1, B1, C1, D1, and E1 and subembodiments contained therein, or a pharmaceutically acceptable salt thereof, are wherein e is N and b and d are CH or C when attached to any one of R³ and R⁴.

### Embodiment J1

In embodiment J1, the compounds of any one of embodiments A1, B1, C1, D1, and E1 and subembodiments contained therein, or a pharmaceutically acceptable salt thereof, are wherein b and e are N and d is CH or C when attached to any one of R³ and R⁴.

### Embodiment K1

In embodiment K1, the compounds of any one of embodiments A1, B1, C1, D1, E1 F1, G1, H1, I1, and J1 and subembodiment contained therein, or a pharmaceutically acceptable salt thereof, are wherein —B(R^{x})(R^{w}) is —B(OH)₂.

### Embodiment L1

In embodiment L1, the compounds of any one of embodiments A1, B1, C1, D1, E1 F1, G1, H1, I1, and J1 and subembodiment contained therein, or a pharmaceutically acceptable salt thereof, are wherein R^{w} and R^{x} are independently selected from hydroxy, alkoxy, —Oaryl (where aryl is optionally substituted with one to three substituents independently selected from alkyl, halo, haloalkyl, cyano, and nitro), —O—(CH₂)OCOR^{a} (where R^{a} is alkyl), or —O-(alk²)OR (where alk² is alkylene and R^{b} is alkyl). Preferably, R^{w} and R^{x} are independently selected from alkoxy, —Oaryl (where aryl is optionally substituted with one to three substituents independently selected from alkyl, halo, haloalkyl, cyano, and nitro), —O—(CH₂)OCOR^{a} (where R^{a} is alkyl), and —O-(alk²)OR (where alk² is alkylene and R^{b} is alkyl, such as methyl, isopropyl, n-propyl, isobutyl, or n-butyl). Preferably, R^{w} and R^{x} are independently hydroxy, alkoxy, or —Ophenyl (where phenyl is optionally substituted with one to three substituents independently selected from alkoxy, halo, haloalkyl, cyano, and nitro); or R^{w} and R^{x} together with the boron atom to which they are attached form a ring of formula (a) or (b):

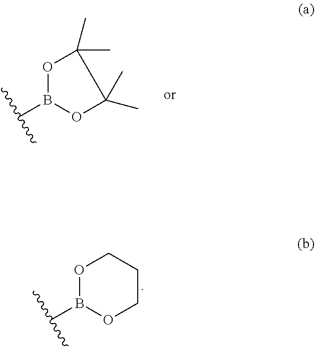

(L1i) In subembodiment (L1i) of embodiment L1, the compounds of embodiment L1, or a pharmaceutically acceptable salt thereof, are wherein R^{w} and R^{x} are independently selected from hydroxy and alkoxy.

(L1ii) In subembodiment (L1ii) of embodiment L1, the compounds of embodiment L1, or a pharmaceutically acceptable salt thereof, are wherein R^{w} and R^{x}together with the boron atom to which they are attached form a ring of formula (a) or (b):

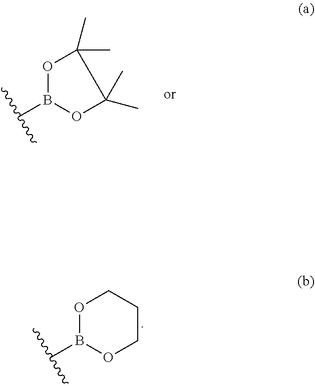

### Embodiment M1

In embodiment M, the compounds of any one of embodiments A1, B1, C1, D1, E1, F1, G1, H1, I1, J1 K1, and L1, and subembodiments contained therein or a pharmaceutically acceptable salt thereof, are wherein Ar is aryl or heteroaryl.

(M1i). In subembodiment (M1i) of embodiment M1, the compounds of embodiment M1, or a pharmaceutically acceptable salt thereof, are wherein Ar is phenyl.

(M1ii). In subembodiment (M1ii) of embodiment M1, the compounds of embodiment M1, or a pharmaceutically acceptable salt thereof, are wherein Ar is phenyl and —B(R^{w})(R^{x}) is attached to carbon of the phenyl ring that is meta to the carbon attaching the phenyl ring to remaining compound of Formula (I) and (Ia1) to (Ig1).

(M1iii). In subembodiment (M1iii) of embodiment M1, the compounds of embodiment M1, or a pharmaceutically acceptable salt thereof, are wherein Ar is phenyl and —B(R^{w})(R^{x}) is attached to carbon on the phenyl ring that is para to the carbon attaching the phenyl ring to remaining compound of Formula (I) and (Ia1) to (Ig1), respectively.

(M1iv). In subembodiment (M1iv) of embodiment M1, the compounds of embodiment M1, or a pharmaceutically acceptable salt thereof, are wherein Ar is heteroaryl.

(M1v). In subembodiment (M1v) of embodiment M1, the compounds of embodiment M1, or a pharmaceutically acceptable salt thereof, are wherein Ar is pyridinyl, pyrimidinyl, pyridazinyl, thienyl, furanyl, thiazolyl, oxazolyl, isoxazolyl, pyrazolyl, triazolyl, oxadiazolyl, or imidazolyl.

(M1vi). In subembodiment (M1vi) of embodiment M1, the compounds of embodiment M1, or a pharmaceutically acceptable salt thereof, are wherein Ar is a six-membered ring such as pyridinyl, pyrimidinyl, or pyridazinyl wherein Q is attached to carbon on the pyridinyl, pyrimidinyl, or pyridazinyl ring that is meta to the carbon attaching the pyridinyl, pyrimidinyl, or pyridazinyl ring to remaining compound of Formula (I), and (Ia1) to (Ig1), respectively.

(M1vii). In subembodiment (M1vii) of embodiment M1, the compounds of embodiment M1, or a pharmaceutically acceptable salt thereof, are those wherein Ar is benzofuranyl, quinolinyl, quinazolinyl, benzimidazolyl, indazolyl, benzotriazolyl, or benzoxazolyl.

### Embodiment N1

In embodiment N1, the compounds of any one of embodiments A1, B1, C1, D1, E1 F1, G1, H1, I1, J1, K1, L1, and M1, and subembodiments contained therein, or a pharmaceutically acceptable salt thereof, are wherein alk is ethnylene.

### Embodiment 01

In embodiment O1, the compounds of any one of embodiments A1, B1, C1, D1, E1 F1, G1, H1, I1, J1, K1, L1, M1, and N1 and subembodiments contained therein, or a pharmaceutically acceptable thereof, are wherein R⁶ is absent, methyl, or isopropyl.

(O1i). In subembodiment (O1i) of embodiment O1, the compounds of embodiment O1, or a pharmaceutically acceptable salt thereof, are those wherein R⁶ is absent.

(O1ii). In subembodiment (O1ii) of embodiment O1, the compounds of embodiment O1, or a pharmaceutically acceptable salt thereof, are wherein R⁶ is methyl or isopropyl.

### Embodiment P1

In embodiment P1, the compounds of any one of embodiments A1, B1, C1, D1, E1 F1, G1, H1, I1, J1, K1, L1, M1, N1, and 01, and subembodiments contained therein, or a pharmaceutically acceptable thereof, are wherein R⁵ is absent, alkyl, hydroxy, halo, acylamino, alkoxyalkylamino, cyano, aminocarbonyl, alkylaminocarbonyl, or dialkylaminocarbonyl.

(P1i). In subembodiment (P1i) of embodiment P, the compounds of embodiment P1, or a pharmaceutically acceptable salt thereof, are wherein R⁵ is absent or alkyl.

(P1ii). In subembodiment (P1ii) of embodiment P1, the compounds of embodiment P1, or a pharmaceutically acceptable salt thereof, are wherein R⁵ is cyano, acylamino, aminocarbonyl, alkylaminocarbonyl, or dialkylaminocarbonyl. Preferably R⁵ is acylamino, aminocarbonyl, methylaminocarbonyl, ethylaminocarbonyl, dimethylaminocarbonyl, or isopropylcarbonyl.

### Embodiment Q1

In embodiment Q1, the compounds of any one of embodiments A, B, C, D, E, E1, E2, E3, F, G, H, I, J, K, L, M, N A1, B1, C1, D1, E1 F1, G1, H1, I1, J1, K1, L1, M1, N1, 01, and P1, and subembodiments contained therein, or a pharmaceutically acceptable thereof, are wherein R¹ and R² are independently absent, methyl, ethyl, methoxy, fluoro, trifluoromethyl, trifluoromethoxy, or cyano.

(Q1i). In subembodiment (Q1i) of embodiment, Q1, the compounds of embodiment Q1, or a pharmaceutically acceptable salt thereof, are wherein R¹ and R² are absent.

### Embodiment R1

(R1i) In embodiment R1i, the compounds of any one of embodiments A1, B1, C1, D1, E1 F1, G1, H1, I1, J1, K1, L1, M1, N1, O1, P1, an Q1 and subembodiments contained therein, or a pharmaceutically acceptable thereof, are wherein R³ and R⁴ are independently absent, alkyl, alkoxy, hydroxy, amino, halo, haloalkyl, or haloalkoxy.

(R1i1). In subembodiment (R1i1) of embodiment (R1i), the compounds of embodiment (R1i), or a pharmaceutically acceptable thereof, are wherein R³ and R⁴ are independently absent, alkoxy, amino, or hydroxy.

(R1i2). In subembodiment (R1i2) of embodiment (R1i), the compounds of embodiment (R1i), or pharmaceutically acceptable thereof, are wherein R³ and R⁴ are independently alkoxy such as methoxy, ethoxy, or propoxy and are attached to to the six membered ring comprising b, d, and e of Formula (I), and (Ia1) to (Ig1) as shown below

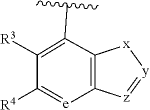

wherein the wavy line denotes the attachment point to the remainder of the molecule.

(R1i3). In subembodiment (R1i3) of embodiment (R1i), the compounds of embodiment (R1i), or pharmaceutically acceptable thereof, are wherein R³ and R⁴ are absent.

(R1i4). In subembodiment (R1i4) of embodiment (R1i), the compounds of embodiment (R1i), or pharmaceutically acceptable thereof, are those wherein when R⁴ is present, R⁴ is attached to the six membered ring comprising b, d, and e of Formula (I), and (Ia1) to (Ig1) as shown below:

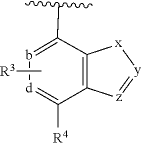

wherein the wavy line denotes the attachment point to the remainder of the molecule.

(R1ii). In embodiment (R1ii), the compounds of any one of embodiments A1, B1, C1, D1, E1 F1, G1, H1, I1, J1, K1, L1, M1, N1, O1, P1, an Q1 and subembodiments contained therein, or a pharmaceutically acceptable thereof, are wherein:
R³ is absent, alkyl, alkoxy, hydroxy, halo, haloalkyl, or haloalkoxy; and
R⁴ is hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one or two substituents where the optional substituents are independently selected from alkyl, hydroxy, alkoxy, halo, haloalkyl, haloalkoxy, and cyano).

In a first subembodiment of embodiment (R1ii), the compounds of embodiment (R1ii), or pharmaceutically acceptable thereof, are wherein R³ is absent, methoxy, ethoxy, or hydroxy, preferably R³ is methoxy or ethoxy; and R⁴ is 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, or methoxy). Preferably, when R³ and R⁴ are present, R³ and R⁴ are attached to the six membered ring comprising b, d, and e of Formula (I), and (Ia1) to (Ig1) as shown below

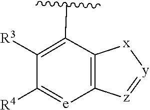

wherein the wavy line denotes the attachment point to the remainder of the molecule.

(R1iii). In embodiment (R1iii), the compounds of any one of embodiments A1, B1, C1, D1, E1 F1, G1, H1, I1, J1, K1, L1, M1, N1, O1, P1, an Q1 and subembodiments contained therein, or a pharmaceutically acceptable thereof, are wherein R³ and R⁴ are independently hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl of phenyloxy and heteroaryl of heteroaryloxy are optionally substituted with one or two substituents, independently selected from alkyl, hydroxy, alkoxy, halo, haloalkyl, haloalkoxy, and cyano).

In a first subembodiment of embodiment (R1iii), the compounds of embodiment (R1iii), or pharmaceutically acceptable thereof, are wherein R³ and R⁴ are independently 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, or methoxy). Preferably, R³ and R⁴ are attached to the six membered ring comprising b, d, and e of Formula (I), and (Ia1) to (Ig1) as shown below:

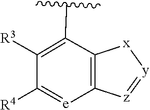

wherein the wavy line denotes the attachment point to the remainder of the molecule.

### General Synthetic Scheme

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (I) where G is NH, O, or S and other groups are as defined in the Summary can be prepared as illustrated and described in Scheme 1 below.

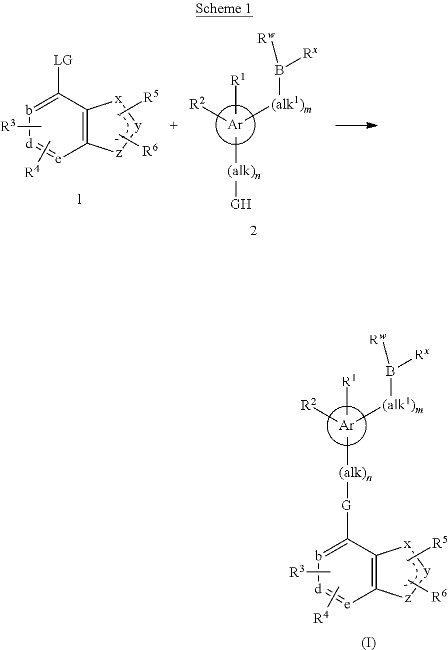

Treatment of a compound of formula 1, where LG is a suitable leaving group such as halo, triflate, and the like and b, d, e, x, y, and z are as defined in the Summary and R³ and R⁴ are as defined in the Summary or a precursor group thereof (e.g., hydroxy is a precursor group of alkoxy etc.), with a boronic acid of the formula 2 where GH is N, O, or S, and Ar, alk, alk1, R¹, R², R^{w} and R^{x} are as defined in the Summary or a precursor group thereof under SN2 reaction conditions provides a compound of Formula (I). The reaction is carried out in the presence of a suitable organic or inorganic base such as potassium carbonate, cesium carbonate, triethylamine, DIEA, and the like, in a suitable organic solvent such as acetonitrile, DMSO, ethanol, and the like, either at room temperature or heating. Compounds of

Compounds of formula 1 and 2 are either commercially available or they can be prepared by methods well known the art. For example, synthesis of a number of compounds of formula 1 are described in Synthetic Examples below.

Additionally, compounds of Formula (I) can be converted to other compounds of Formula (I) by method well known in the art. Some such methods are described in Synthetic Examples below.

### Testing

The ENPP1 inhibitory activity of the compounds of the present disclosure can be tested using the in vitro assays described in Biological Examples 1 and 2 below.

### Administration and Pharmaceutical Composition

In general, the compounds of this disclosure will be administered in a therapeutically effective amount by any of the accepted modes of administration for agents that serve similar utilities. Therapeutically effective amounts of compounds this disclosure may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the patient, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of this disclosure in combination with at least one pharmaceutically acceptable excipient. Acceptable excipients are non-toxic, aid administration, and do not adversely affect the therapeutic benefit of the compound of this disclosure. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this disclosure in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 20th ed., 2000).

The level of the compound in a formulation can vary within the full range employed by those skilled in the art. Typically, the formulation will contain, on a weight percent (wt. %) basis, from about 0.01-99.99 wt. % of a compound of this disclosure based on the total formulation, with the balance being one or more suitable pharmaceutical excipients. For example, the compound is present at a level of about 1-80 wt. %.

The compounds of this disclosure may be used in combination with one or more other drugs in the treatment of diseases or conditions for which compounds of this disclosure or the other drugs may have utility. Such other drug(s) may be administered, by a route and in an amount commonly used therefore, contemporaneously or sequentially with a compound of the present disclosure. When a compound of this disclosure is used contemporaneously with one or more other drugs, a pharmaceutical composition in unit dosage form containing such other drugs and the compound of the present disclosure is preferred. However, the combination therapy may also include therapies in which the compound of this disclosure and one or more other drugs are administered on different overlapping schedules. It is also contemplated that when used in combination with one or more other active ingredients, the compounds of the present disclosure and the other active ingredients may be used in lower doses than when each is used singly. Accordingly, the pharmaceutical compositions of the present disclosure also include those that contain one or more other drugs, in addition to a compound of the present disclosure.

The above combinations include combinations of a compound of this disclosure not only with one other drug, but also with two or more other active drugs. Likewise, a compound of this disclosure may be used in combination with other drugs that are used in the prevention, treatment, control, amelioration, or reduction of risk of the diseases or conditions for which a compound of this disclosure is useful. Such other drugs may be administered, by a route and in an amount commonly used therefore, contemporaneously or sequentially with a compound of the present disclosure. When a compound of this disclosure is used contemporaneously with one or more other drugs, a pharmaceutical composition containing such other drugs in addition to the compound of this disclosure can be used. Accordingly, the pharmaceutical compositions of the present disclosure also include those that also contain one or more other active ingredients, in addition to a compound of this disclosure. The weight ratio of the compound of this disclosure to the second active ingredient may be varied and will depend upon the effective dose of each ingredient. Generally, an effective dose of each will be used.

Where the subject in need is suffering from or at risk of suffering from cancer, the subject can be treated with a compound of this disclosure in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec™), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™., such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents for use in combination with a compound of this disclosure include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; antibodies (e.g., rituxan); MET inhibitor such as foretinib, carbozantinib, or crizotinib; VEGFR inhibitor such as sunitinib, sorafenib, regorafinib, lenvatinib, vandetanib, carbozantinib, axitinib; EGFR inhibitor such as afatinib, brivanib, carbozatinib, erlotinib, gefitinib, neratinib, lapatinib; PI3K inhibitor such as XL147, XL765, BKM120 (buparlisib), GDC-0941, BYL719, IPI145, BAY80-6946. BEX235 (dactolisib), CAL101 (idelalisib), GSK2636771, TG100-115; MTOR inhibitor such as rapamycin (sirolimus), temsirolimus, everolimus, XL388, XL765, AZD2013, PF04691502, PKI-587, BEZ235, GDC0349; MEK inhibitor such as AZD6244, trametinib, PD184352, pimasertinib, GDC-0973, AZD8330; and proteasome inhibitor such as carfilzomib, MLN9708, delanzomib, or bortezomib.

Other anti-cancer agents that can be employed in combination with a compound of this disclosure include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or Ril2), interferon alfa-2a; interferon alfa-2b; interferon alfa-n1; interferon alfa-n3; interferon beta-la; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with a compound of the disclosure such as 8-(3-(4-acryloylpiperazin-1-yl)propyl)-6-(2,6-dichloro-3,5-dimethoxyphenyl)-2-(methylamino)pyrido[2,3-d]pyrimidin-7(8H)-one used to determine the anti-tumor activity in HGS and RT4 tumor models (Example 4 below: In HGS model, vehicle dosed group reached tumor size 645dosing at day 42 after inoculation whereas for animals treated with 20/kg of compound, the tumor size was 55 mm3 showing significant antitumor activity and induced tumor regression), include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; Bfgf inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A; diethylstilbestrol; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R.sub.11 retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a compound of this disclosure include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., cytarabine), purine analogs (e.g., mercaptopurine, thioguanine, pentostatin).

Examples of natural products useful in combination with a compound of this disclosure include but are not limited to vinca alkaloids (e.g., vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination a compound of this disclosure) include, but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxuridine, cytarabine), purine analogs (e.g., mercaptopurine, thioguanine, pentostatin.

Examples of hormones and antagonists useful in combination a compound of this disclosure include, but are not limited to, adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with an irreversible Btk inhibitor compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B. Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Further examples of anti-cancer agents for use in combination with a compound of this disclosure include immune checkpoint inhibitors. Exemplary immune checkpoint inhibitors include inhibitors (small molecules or biologics) against immune checkpoint molecules such as CD27, CD28, CD40, CD122, CD96, CD73, CD39, CD47, OX40, GITR, CSF1R, JAK, PI3K delta, PI3K gamma, TAM kinase, arginase, CD137 (also known as 4-1iB), ICOS, A2AR, A2BR, HIF-2α, B7-H3, B7-H4, BTLA, CTLA-4, LAG3, TIM3, VISTA, CD96, TIGIT, PD-1, PD-L1 and PD-L2. In some embodiments, the immune checkpoint molecule is a stimulatory checkpoint molecule selected from CD27, CD28, CD40, ICOS, OX40, GITR, CD137 and STING. In some embodiments, the immune checkpoint molecule is an inhibitory checkpoint molecule selected from B7-H3, B7-H4, BTLA, CTLA-4, IDO, TDO, Arginase, KIR, LAG3, PD-1, TIM3, CD96, TIGIT and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab).

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of OX40, e.g., an anti-OX40 antibody or OX40L fusion protein. In some embodiments, the anti-OX40 antibody is MED10562 or, INCAGN01949, GSK2831781, GSK-3174998, MOXR-0916, PF-04518600 or LAG525. In some embodiments, the OX40L fusion protein is MEDI6383 EXAMPLES

The following preparations of compounds of Formula (I) are given to enable those skilled in the art to more clearly understand and to practice the present disclosure. They should not be considered as limiting the scope of the disclosure, but merely as being illustrative and representative thereof.

All solvents used were commercially available and were used without further purification. Reactions were typically run using anhydrous solvents under an inert atmosphere of nitrogen.

¹H spectra were recorded at 400 MHz or 300 MHz for proton on a Bruker 400 NMR Spectrometer equipped with a Bruker 400 BBO probe or Bruker BBFO ULTRASHIELD™300 AVANCE III, respectively. All deuterated solvents contained typically 0.03% to 0.05% v/v tetramethylsilane, which was used as the reference signal (set at 6 0.00 for both ¹H and ¹³C).

LCMS analyses were performed on a SHIMADZU LCMS consisting of an UFLC 20-AD and LCMS 2020 MS detector. The Diode Array Detector was scanned from 190-400 nm. The mass spectrometer was equipped with an electrospray ion source (ESI) operated in a positive or negative mode. The mass spectrometer was scanned between m/z 90-900 with a scan time from 0.5 to 3.0 s.

HPLC analyses were performed on a SHIMADZU UFLC with two LC20 AD pump and a SPD-M20A Photodiiode Array Detector. The column used was an XBridge C18, 3.5 μm, 4.6×100 mm. A linear gradient was applied, starting at 90% A (A: 0.05% TFA in water) and ending at 95% B (B: 0.05% TFA in MeCN) over 10 min with a total run time of 15 min. The column temperature was at 40° C. with the flow rate of 1.5 mL/min. The Diode Array Detector was scanned from 200-400 nm.

Thin layer chromatography (TLC) was performed on Alugram® (Silica gel 60 F254) from Mancherey-Nagel and UV was typically used to visualize the spots. Additional visualization methods were also employed in some cases. In these cases the TLC plate was developed with iodine (generated by adding approximately 1 g of I₂ to 10 g silica gel and thoroughly mixing), ninhydrin (available commercially from Aldrich), or Magic Stain (generated by thoroughly mixing 25 g (NH₄)₆Mo₇O₂₄·4H₂O, 5 g (NH₄)₂Ce(IV)(NO₃)₆ in 450 mL water and 50 mL concentrated H₂SO₄) to visualize the compound. Flash chromatography was performed using 40-63 μm (230-400 mesh) silica gel from Silicycle following analogous techniques to those disclosed in Still, W. C.; Kahn, M.; and Mitra, M. Journal of Organic Chemistry, 1978, 43, 2923. Typical solvents used for flash chromatography or thin layer chromatography were mixtures of chloroform/methanol, dichloromethane/methanol, ethyl acetate/methanol and hexanes/ethyl acetate.

### SYNTHETIC EXAMPLES

All solvents used were commercially available and were used without further purification. Reactions were typically run using anhydrous solvents under an inert atmosphere of nitrogen.

¹H spectra were recorded at 400 MHz or 300 MHz for proton on a Bruker 400 NMR Spectrometer equipped with a Bruker 400 BBO probe or Bruker BBFO ULTRASHIELD™300 AVANCE III, respectively. All deuterated solvents contained typically 0.03% to 0.05% v/v tetramethylsilane, which was used as the reference signal (set at d 0.00 for both ¹H and ¹³C).

LCMS analyses were performed on a SHIMADZU LCMS consisting of an UFLC 20-AD and LCMS 2020 MS detector. The Diode Array Detector was scanned from 190-400 nm. The mass spectrometer was equipped with an electrospray ion source (ESI) operated in a positive or negative mode. The mass spectrometer was scanned between m/z 90-900 with a scan time from 0.5 to 3.0 s.

HPLC analyses were performed on a SHIMADZU UFLC with two LC20 AD pump and a SPD-M20A Photodiiode Array Detector. The column used was an XBridge C18, 3.5 μm, 4.6×100 mm. A linear gradient was applied, starting at 90% A (A: 0.05% TFA in water) and ending at 95% B (B: 0.05% TFA in MeCN) over 10 min with a total run time of 15 min. The column temperature was at 40° C. with the flow rate of 1.5 mL/min. The Diode Array Detector was scanned from 200-400 nm.

Thin layer chromatography (TLC) was performed on Alugram® (Silica gel 60 F254) from Mancherey-Nagel and UV was typically used to visualize the spots. Additional visualization methods were also employed in some cases. In these cases the TLC plate was developed with iodine (generated by adding approximately 1 g of I₂ to 10 g silica gel and thoroughly mixing), ninhydrin (available commercially from Aldrich), or Magic Stain (generated by thoroughly mixing 25 g (NH₄)₆Mo₇O₂₄·4H₂O, 5 g (NH₄)₂Ce(IV)(NO₃)₆ in 450 mL water and 50 mL concentrated H₂SO₄) to visualize the compound. Flash chromatography was performed using 40-63 μm (230-400 mesh) silica gel from Silicycle following analogous techniques to those disclosed in Still, W. C.; Kahn, M.; and Mitra, M. Journal of Organic Chemistry, 1978, 43, 2923. Typical solvents used for flash chromatography or thin layer chromatography were mixtures of chloroform/methanol, dichloromethane/methanol, ethyl acetate/methanol and hexanes/ethyl acetate.

### Example 1

### Synthesis of 4-[([7-carbamoyl-5H-pyrrolo[3,2-d]pyrimidin-4-yl]amino)methyl]phenylboronic acid

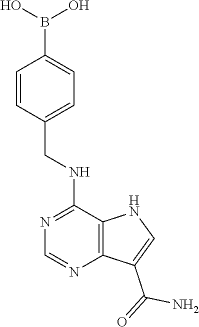 [SMILES: NC(=O)c1c[nH]c2c(NCc3ccc(B(O)O)cc3)ncnc12]

### Step 1: ethyl 4-chloro-5H-pyrrolo[3,2-d]pyrimidine-7-carboxylate

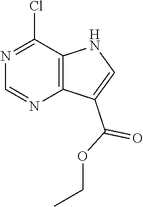

A solution of ethyl 4-hydroxy-5H-pyrrolo[3,2-d]pyrimidine-7-carboxylate (1.00 g) in phosphorus oxychloride (15 mL) was stirred for 3 h at 90° C. After cooling to room temperature and concentration under reduced pressure, the residue was purified by column chromatography on silica gel (eluent: petroleum ether-ethyl acetate 100%, 1:1) to give ethyl 4-chloro-5H-pyrrolo[3,2-d]pyrimidine-7-carboxylate (0.6 g, 55%) as a grey solid.

### Step 2: 4-([[7-(ethoxycarbonyl)-5H-pyrrolo[3,2-d]pyrimidin-4-yl]amino]methyl)-phenylboronic acid

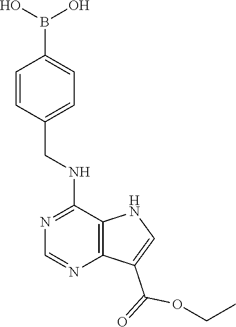 [SMILES: CCOC(=O)c1c[nH]c2c(NCc3ccc(B(O)O)cc3)ncnc12]

To a solution of ethyl 4-chloro-5H-pyrrolo[3,2-d]pyrimidine-7-carboxylate (0.60 g, 2.659 mmol, 1.00 equiv) in ACN (15.00 mL) were added 4-(aminomethyl)phenylboronic acid hydrochloride (0.60 g, 3.191 mmol, 1.20 equiv) and K₂CO₃ (0.74 g, 5.318 mmol, 2.00 equiv). After stirring for 2 h at 80° C., the reaction mixture was concentrated under reduced pressure, poured into water and extracted with EA. The organic layers were combined, dried over anhydrous sodium sulfate, filtered and evaporated under reduced pressure. The residue was purified by column chromatography (silica gel, eluent: DCM/MeOH 98:2) to give the title compound (0.6 g, 66%).

### Step 3: 4-[([7-carbamoyl-5H-pyrrolo[3,2-d]pyrimidin-4-yl]amino)methyl]phenylboronic acid

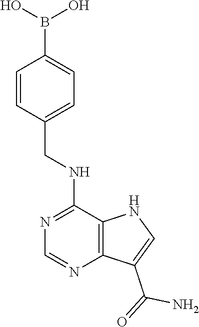 [SMILES: NC(=O)c1c[nH]c2c(NCc3ccc(B(O)O)cc3)ncnc12]

A mixture of 4-([[7-(ethoxycarbonyl)-5H-pyrrolo[3,2-d]pyrimidin-4-yl]amino]methyl)phenylboronic acid (100 mg, 0.294 mmol, 1.00 equiv) in dioxane (3.00 mL, 35.412 mmol, 120.45 equiv) was added to concentrated ammonia (10 mL). After stirring overnight at 80° C., the reaction mixture was concentrated under reduced pressure. The residue was purified by Column: XBridge Shield RP18 OBD Column, 19*250 mm, 10 um; Mobile Phase A: Water (10 mmol/L NH₄HCO₃), Mobile Phase B: ACN; Flow rate: 25 mL/min; Gradient: 18 B to 21 B in 7 min; 254/220 nm; to give the title compound (13.9 mg, 14%) as a white solid. MS (ESI, pos. ion) m/z: 312.3 (M+1). ¹H NMR (300 MHz, DMSO/-d₆/D₂O, ppm) δ 8.31 (s, 1H), 8.00 (s, 1H), 7.78-7.69 (m, 2H), 7.34 (d, J=8.0 Hz, 2H), 4.73 (s, 2H).

### Example 2

### Synthesis of 4-[([7-cyano-5H-pyrrolo[3,2-d]pyrimidin-4-yl]amino)methyl]phenylboronic acid

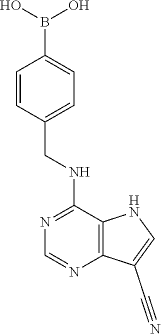 [SMILES: N#Cc1c[nH]c2c(NCc3ccc(B(O)O)cc3)ncnc12]

### Step 1: 4-hydroxy-5H-pyrrolo[3,2-d]pyrimidine-7-carboxamide

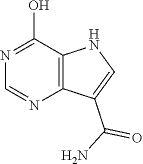 [SMILES: NC(=O)c1c[nH]c2c(O)ncnc12]

A solution of ethyl 4-hydroxy-5H-pyrrolo[3,2-d]pyrimidine-7-carboxylate (200.00 mg, 207 mmol, 1.00 equiv) in concentrated ammonia (10 mL) was stirred overnight at 90° C. After cooling down to room temperature and concentration under reduced pressure, the residue was purified by column chromatography on silica gel (eluent: petroleum ether-ethyl acetate 100%, 1:3) to give the title compound (150 mg, 77%) as an off-white solid.

### Step 2: 4-chloro-5H-pyrrolo[3,2-d]pyrimidine-7-carbonitrile

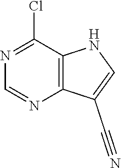 [SMILES: N#Cc1c[nH]c2c(Cl)ncnc12]

A solution of 4-hydroxy-5H-pyrrolo[3,2-d]pyrimidine-7-carboxamide (150 mg, 0.842 mmol, 1.00 equiv) in phosphorus oxychloride (5 mL) was heated at reflux for 3 h. After cooling down to room temperature and concentration under reduced pressure, the residue was purified by column chromatography on silica gel (eluent: petroleum ether-ethyl acetate 100%, 1:1) to give the title compound (130 mg, crude) as an off-white solid.

### Step 3: 4-[([7-cyano-5H-pyrrolo[3,2-d]pyrimidin-4-yl]amino)methyl]phenylboronic acid

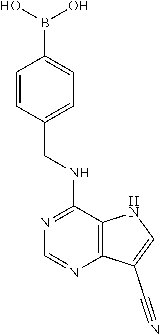 [SMILES: N#Cc1c[nH]c2c(NCc3ccc(B(O)O)cc3)ncnc12]

The title compound was synthesized by the same method as described in example 1, step 2 except 4-chloro-5H-pyrrolo[3,2-d]pyrimidine-7-carbonitrile (80 mg, crude) was used. The crude product was purified by prep-HPLC with the following conditions (Column: Sunfire prep C18 column, 30*150, 5 um; Mobile Phase A: Water (0.1% FA), Mobile Phase B: ACN; Flow rate: 60 mL/min; Gradient: 5 B to 25 B in 7 min; 254/220 nm) to afford the title compound (8.1 mg) as a white solid. MS (ESI, pos. ion) m/z: 294.2 (M+1). ¹H NMR (300 MHz, DMSO/-d₆/D₂0, ppm) δ 8.24 (s, 1H), 8.16 (s, 1H), 7.70 (d, J=7.5 Hz, 2H), 7.33 (d, J=7.5 Hz, 2H), 4.69 (s, 2H).

### Example 3

### Synthesis of 4-([[7-(methylcarbamoyl)-5H-pyrrolo[3,2-d]pyrimidin-4-yl]amino]-methyl)phenylboronic acid; formic acid salt

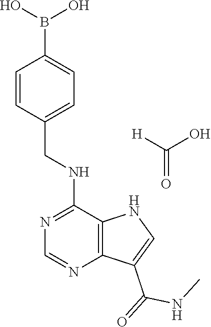

A mixture of 4-([[7-(ethoxycarbonyl)-5H-pyrrolo[3,2-d]pyrimidin-4-yl]amino]methyl)phenylboronic acid (150 mg, 1 equiv) in CH₃NH₂ (30% in MeOH) (5.00 mL) was stirred overnight at 80° C. After cooling to room temperature, the resulting mixture was concentrated under reduced pressure. The crude product was purified by prep-HPLC with the following conditions (Column: XBridge Shield RP18 OBD Column, 19*250 mm, 10 um; Mobile Phase A: Water (0.1% FA), Mobile Phase B: ACN; Flow rate: 25 mL/min; Gradient: 9 B to 19 B in 7 min; 254/220 nm;) to afford the title compound (15.1 mg, 8.3%) as yellow solid. MS (ESI, pos. ion) m/z: 326.0 (M+1). ¹H NMR (300 MHz, DMSO-d₆, ppm) δ 11.47 (s, 1H), 8.42 (d, J=4.9 Hz, 1H), 8.33 (s, 1H), 8.17 (s, 1H), 8.02 (d, J=13.7 Hz, 3H), 7.78 (d, J=7.9 Hz, 3H), 7.36 (d, J=7.7 Hz, 2H), 4.76 (d, J=5.6 Hz, 2H), 2.88 (d, J=4.8 Hz, 3H).

### Example 4

### Synthesis of 4-[(7H-purin-6-ylamino)methyl]phenylboronic acid

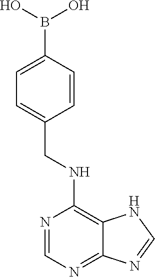 [SMILES: OB(O)c1ccc(CNc2ncnc3nc[nH]c23)cc1]

The title compound was synthesized by the same method as described in example 1, step 2 except 6-chloropurine (100 mg, 0.647 mmol) was used. The crude product was purified by prep-HPLC with the following conditions (Column: XBridge Prep OBD C18 Column, 19*250 mm, 5 um; Mobile Phase A: Water (0.1% FA), Mobile Phase B: ACN; Flow rate: 25 mL/min; Gradient: 5 B to 23 B in 9 min; 254/220 nm) to afford the title compound (82.0 mg, 46%) as a white solid. MS (ESI, pos. ion) m/z: 270.3 (M+1). ¹H NMR (300 MHz, DMSO-d₆, ppm) δ 12.93 (s, 1H), 8.13 (d, J=16.3 Hz, 3H), 7.94 (s, 2H), 7.70 (d, J=7.7 Hz, 2H), 7.29 (d, J=7.7 Hz, 2H), 4.72 (s, 2H).

### Example 5

### Synthesis of 4-[[(7-methylpurin-6-yl)amino]methyl]phenylboronic acid

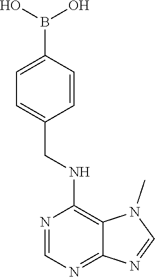 [SMILES: Cn1cnc2ncnc(NCc3ccc(B(O)O)cc3)c21]

The title compound was synthesized by the same method as described in example 1, step 2 except 6-chloro-7-methylpurine (100 mg, 0.593 mmol) was used. The crude product was purified by prep-HPLC with the following conditions (Column: XBridge Prep OBD C18 Column, 19*250 mm, 5 um; Mobile Phase A: Water (0.1% FA), Mobile Phase B: ACN; Flow rate: 25 mL/min; Gradient: 5 B to 23 B in 9 min; 254/220 nm) to afford the title compound (46.8 mg, 27%) as a white solid. MS (ESI, pos. ion) m/z: 284.2 (M+1). ¹H NMR (300 MHz, DMSO-d₆, ppm) δ 8.19 (d, J=3.3 Hz, 2H), 7.98 (s, 1H), 7.72 (d, J=7.5 Hz, 2H), 7.54 (q, J=7.7, 6.8 Hz, 1H), 7.33 (d, J=7.4 Hz, 2H), 4.74 (d, J=5.9 Hz, 2H), 4.08 (d, J=4.3 Hz, 3H).

### Example 6

### Synthesis of 4-([1H-pyrazolo[4,3-d]pyrimidin-7-ylamino]methyl)phenylboronic acid

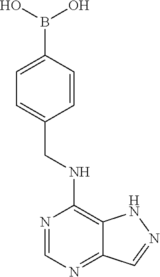 [SMILES: OB(O)c1ccc(CNc2ncnc3cn[nH]c23)cc1]

To a stirred solution of 7-chloro-1H-pyrazolo[4,3-d]pyrimidine (80 mg, 0.518 mmol, 1.00 equiv) in DMSO (4.00 mL) were added 4-(aminomethyl)phenylboronic acid hydrochloride (291 mg, 1.554 mmol, 3.00 equiv) and TEA (157 mg, 1.554 mmol, 3.00 equiv). After stirring for 16 h at 80° C., the mixture was concentrated under reduced pressure. The crude product was purified by prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 2 B to 10 B in 8 min, 254/220 nm) to afford the title compound (37.7 mg, 26%) as a white solid. MS (ESI, pos. ion) m/z: 270.2 (M+1). ¹H NMR (300 MHz, DMSO-d₆, ppm) δ 13.41 (s, 1H), 8.69 (s, 1H), 8.23 (s, 1H), 8.16 (s, 1H), 7.99 (s, 2H), 7.80-7.71 (m, 2H), 7.32 (d, J=7.7 Hz, 2H), 4.75 (d, J=5.9 Hz, 2H).

### Example 7

### Synthesis of 4-[([1-methylpyrazolo[4,3-d]pyrimidin-7-yl]amino)methyl]phenylboronic acid

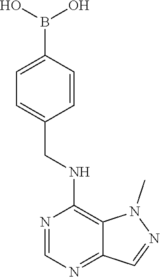 [SMILES: Cn1ncc2ncnc(NCc3ccc(B(O)O)cc3)c21]

The title compound was synthesized by the same method as described in example 1, step 2 except 7-chloro-1-methylpyrazolo[4,3-d]pyrimidine (100.00 mg, 0.593 mmol) was used. The crude product was purified by prep-HPLC with the following conditions (Column: XBridge Shield RP18 OBD Column, 19*250 mm, 10 um; Mobile Phase A: Water (0.1% FA), Mobile Phase B: ACN; Flow rate: 25 mL/min; Gradient: 5 B to 30 B in 7 min; 254/220 nm) to afford the title compound (66.5 mg, 39%) as a white solid. MS (ESI, pos. ion) m/z: 284.1 (M+1). HTEM-NMR (300 MHz, DMSO-d₆ ppm) δ 8.15 (s, 1H), 7.90 (s, 1H), 7.68 (d, J=7.8 Hz, 2H), 7.34 (d, J=7.6 Hz, 2H), 4.77 (s, 2H), 4.25 (s, 3H).

### Example 8

### Synthesis of 4-([[2-methyl-7-(methylcarbamoyl)-5H-pyrrolo[3,2-d]pyrimidin-4-yl]amino]methyl)-phenylboronic acid

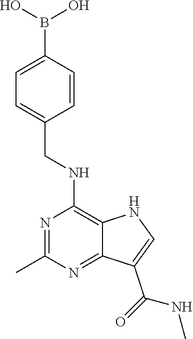

### Step 1: 2,4-diethyl 3-amino-1H-pyrrole-2,4-dicarboxylate

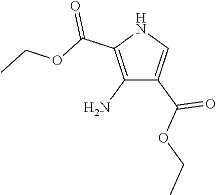 [SMILES: CCOC(=O)c1c[nH]c(C(=O)OCC)c1N]

To a solution of 1,3-diethyl 2-aminopropanedioate hydrochloride (5.00 g, 23.625 mmol) in EtOH (100.00 mL) were added ethyl (2E)-2-cyano-3-ethoxyprop-2-enoate (4.80 g, 28.350 mmol) and sodium ethoxide (3.22 g, 47.250 mmol, 2.00 equiv) at room temperature. The resulting mixture was stirred overnight at 80° C. After cooled to room temperature, the mixture was concentrated under reduced pressure and purified by CombiFlash with the following conditions (MeOH in water, 10% to 100% gradient in 30 min; detector, UV 254/220 nm.) to afford the title compound (3.1 g, 58%) as a grey solid.

### Step 2: 2,4-diethyl 3-ethanimidamido-1H-pyrrole-2,4-dicarboxylate

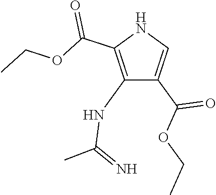 [SMILES: CCOC(=O)c1c[nH]c(C(=O)OCC)c1NC(C)=N]

A solution of 2,4-diethyl 3-amino-1H-pyrrole-2,4-dicarboxylate (2.90 g, 12.819 mmol, 1.00 equiv) in ACN (100 mL) was treated with 10 mL HCl (gas, 4 M in 1,4-dioxane) at 0° C. under nitrogen atmosphere. After stirring for 18 h at 50° C., the resulting mixture was concentrated under reduced pressure, diluted with water (30 mL) and neutralized to pH 7.0 with NaOH (2 M). The precipitated solid was collected by filtration and dried in vacuo to give 2 the title compound (2.0 g, 58%) as a white solid.

### Step 3: 2-methyl-4-oxo-3H,5H-pyrrolo[3,2-d]pyrimidine-7-carboxylic acid

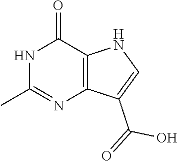

A solution of 2,4-diethyl 3-ethanimidamido-1H-pyrrole-2,4-dicarboxylate (500 mg, 1.00 equiv) in NaOH solution (6 M in water, 10.00 mL) was stirring for 4 h at 90° C. After cooling to room temperature, the resulting mixture was basified to pH 4-5 with HCl (3 M). The precipitated solid was collected by filtration and dried in vacuo to give the title compound (300 mg, 83%) as an off-white solid.

### Step 4: N,2-dimethyl-4-oxo-3H,5H-pyrrolo[3,2-d]pyrimidine-7-carboxamide

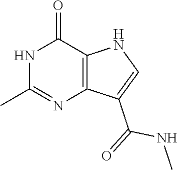

To a solution of 2-methyl-4-oxo-3H,5H-pyrrolo[3,2-d]pyrimidine-7-carboxylic acid (0.85 g, 4.400 mmol, 1.00 equiv) in DMF (20 mL) were added TEA (1.34 g, 13.200 mmol, 3.00 equiv), methanamine, hydrochloride (0.59 g, 8.801 mmol, 2.00 equiv) and T3P (5.60 g, 8.800 mmol, 2.00 equiv, 50%). The resulting mixture was stirred for 24 hours at 50° C. After cooling down to room temperature, the reaction mixture was concentrated under reduced pressure. The residue was purified by silica gel column chromatography, eluted with MeOH/DCM (1:20) to give the title compound (300 mg, 33%) as an white solid.

### Step 5: 4-chloro-N,2-dimethyl-5H-pyrrolo[3,2-d]pyrimidine-7-carboxamide

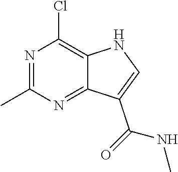

To a mixture of N,2-dimethyl-4-oxo-3H,5H-pyrrolo[3,2-d]pyrimidine-7-carboxamide (150 mg, 0.727 mmol, 1.00 equiv) in SOCl₂ (5.00 mL) was added DMF (0.10 mL). The resulting mixture was stirred for 2 hours at 80° C. After cooling down to room temperature, the reaction mixture was concentrated under reduced pressure. The residue was purified by CombiFlash with the following conditions ACN in water, 5% to 30% gradient in 20 min; detector, UV 254/220 nm.) to give the title compound (35 mg, 21%) as an off-white solid.

### Step 6: 4-([[2-methyl-7-(methylcarbamoyl)-5H-pyrrolo[3,2-d]pyrimidin-4-yl]amino]methyl)-phenyl-boronic acid

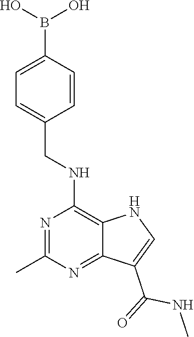

To a solution of 4-chloro-N,2-dimethyl-5H-pyrrolo[3,2-d]pyrimidine-7-carboxamide (32 mg, 0.142 mmol, 1.00 equiv) in DMSO (3.00 mL) were added TEA (43 mg, 0.426 mmol, 3.00 equiv) and 4-(aminomethyl)phenylboronic acid hydrochloride (40 mg, 0.21 mmol, 1.50 equiv). The resulting mixture was stirred for 3 h at 80° C. After cooling down to room temperature, the reaction mixture was concentrated under reduced pressure. The crude product was purified by prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 6% B to 16% B in 7 min, 16% B; Wave Length: 254; 220 nm) to afford the title compound d (22.0 mg, 43%) as a white solid. MS (ESI, pos. ion) m/z: 340.0 (M+1). ¹H NMR (400 MHz, DMSO-d₆ ppm) δ 11.33 (s, 1H), 8.51 (m, 1H), 8.21-7.85 (m, 3H), 7.84-7.74 (m, 2H), 7.66-7.55 (m, 1H), 7.36 (d, J=7.8 Hz, 2H), 4.73 (dd, J=9.2, 5.3 Hz, 2H), 2.88 (d, J=4.7 Hz, 3H), 2.49 (s, 3H).

### Example 9

### Synthesis of 4-([[7-(dimethylcarbamoyl)-5H-pyrrolo[3,2-d]pyrimidin-4-yl]amino]methyl)-phenylboronic acid

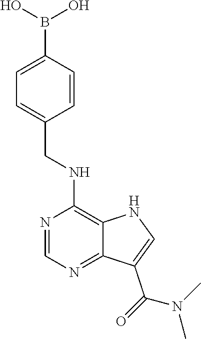 [SMILES: CN(C)C(=O)c1c[nH]c2c(NCc3ccc(B(O)O)cc3)ncnc12]

### Step 1: 4-chloro-5H1-pyrrolo[3,2-d]pyrimidine-7-carboxylic acid

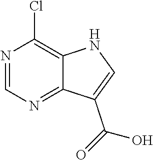 [SMILES: O=C(O)c1c[nH]c2c(Cl)ncnc12]

To a solution of ethyl 4-chloro-5H-pyrrolo[3,2-d]pyrimidine-7-carboxylate (2.00 g, 8.864 mmol, 1.00 equiv) in THE (30 mL) and water (10 mL, 777.113 mmol, 1.00 equiv) was added with lithium hydroxide (425 mg, 17.728 mmol, 2.00 equiv) at room temperature. After stirring for overnight at 60° C., the reaction mixture was cooled to 0° C. and adjusting the PH to 3-4 with 1 N HCl. The precipitated solid was collected by filtration, washed with water and dried in vacuo to give the title compound (1.13 g, 63%) as a white solid.

### Step 2: 4-chloro-N,N-dimethyl-5H-pyrrolo[3,2-d]pyrimidine-7-carboxamide

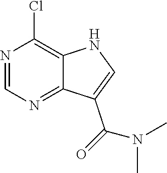

The title compound was synthesized by the same method as described in example 8, step except 4-chloro-5H-pyrrolo[3,2-d]pyrimidine-7-carboxylic acid (120 mg, 0.607 mmol) and dimethylamine (0.3 mL) were used. The title compound (70 mg, 51.30%) was obtained as a white solid.

### Step 3: 4-([[7-(dimethylcarbamoyl)-5H-pyrrolo[3,2-d]pyrimidin-4-yl]amino]methyl)phenyl-boronic acid

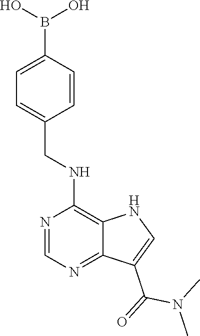 [SMILES: CN(C)C(=O)c1c[nH]c2c(NCc3ccc(B(O)O)cc3)ncnc12]

To a stirred solution of 4-chloro-N,N-dimethyl-5H-pyrrolo[3,2-d]pyrimidine-7-carboxamide (70 mg, 0.31 mmol, 1.00 equiv) in DMSO (3 mL) were added 4-(aminomethyl)phenylboronic acid hydrochloride (117 mg, 0.62 mmol, 2.00 equiv) and DIEA (121 mg, 0.94 mmol, 3.00 equiv) at room temperature and stirring for 12 h at 80° C. The crude product was purified by prep-HPLC with the following conditions (Column: XBridge Prep OBD C18 Column, 19*250 mm, 5 um; Mobile Phase A: Water (10 mmoL/L NH₄HCO₃+0.1% NH₃·H₂O), Mobile Phase B: ACN; Flow rate: 25 mL/min; Gradient: 11 B to 25 B in 7 min, 25 B to B in 10 min, 254/220 nm) to afford the title compound (35.4 mg, 32%) as a white solid. MS (ESI, pos. ion) m/z: 340.0 (M+1). ¹H NMR (300 MHz, DMSO-d₆, ppm) δ 11.33 (s, 1H), 8.25 (s, 1H), 8.01 (s, 2H), 7.82-7.73 (m, 3H), 7.60 (t, J=5.6 Hz, 1H), 7.36 (d, J=7.8 Hz, 2H), 4.74 (d, J=5.5 Hz, 2H), 3.03 (s, 6H).

### Example 10

### Synthesis of 4-([[7-(methylcarbamoyl)thieno[3,2-d]pyrimidin-4-yl]amino]methyl)-phenylboronic acid

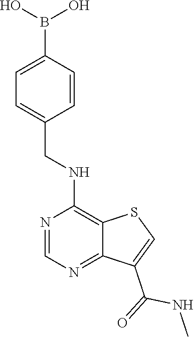 [SMILES: CNC(=O)c1csc2c(NCc3ccc(B(O)O)cc3)ncnc12]

### Step 1: 4-chloro-N-methylthieno[3,2-d]pyrimidine-7-carboxamide

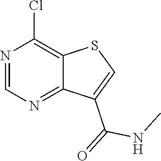

The title compound was synthesized by the same method as described in example 8, step except 4-chlorothieno[3,2-d]pyrimidine-7-carboxylic acid (150 mg, 0.70 mmol) was used. The crude product was purified by CombiFlash with the following conditions ACN in water, 5% to 20% gradient in 20 min; detector, UV 254/220 nm.) to give the title compound (70 mg, 44%) as a yellow solid. MS (ESI, pos. ion) m/z: 227.9 (M+1).

### Step 2: 4-([[7-(methylcarbamoyl)thieno[3,2-d]pyrimidin-4-yl]amino]methyl)phenylboronic acid

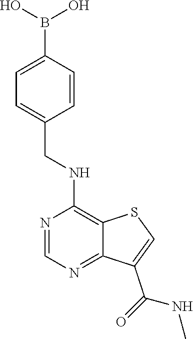 [SMILES: CNC(=O)c1csc2c(NCc3ccc(B(O)O)cc3)ncnc12]

A mixture of 4-chloro-N-methylthieno[3,2-d]pyrimidine-7-carboxamide (1 equiv.), TEA (3 equiv.) and 4-(aminomethyl)phenylboronic acid hydrochloride (1.50 equiv) was heated overnight at 80° C. After cooling the reaction mixture to room temperature, the reaction mixture was concentrated. The crude product was purified by prep-HPLC with the following conditions (Column: Sunfire prep C18 column, 30*150 mm, 5 m; Mobile Phase A: Water (0.1% FA), Mobile Phase B: ACN; Flow rate: 60 mL/min; Gradient: 15% B to 28% B in 7 min, 28% B; Wave Length: 254/220 nm;) to afford the title compound (41.6 mg, 46%) as a white solid. MS (ESI, pos. ion) m/z: 343.2 (M+1). ¹H NMR (300 MHz, DMSO-d₆, ppm) δ 9.44 (d, J=4.9 Hz, 1H), 8.82 (t, J=5.9 Hz, 1H), 8.74 (s, 1H), 8.56 (s, 1H), 7.99 (s, 2H), 7.74 (d, J=7.7 Hz, 2H), 7.31 (d, J=7.7 Hz, 2H), 4.78 (d, J=5.9 Hz, 2H), 2.92 (d, J=4.7 Hz, 3H).

### Example 11

### Synthesis of 4-([[7-(ethylcarbamoyl)-5H-pyrrolo[3,2-d]pyrimidin-4-yl]amino]methyl)-phenylboronic acid; formic acid

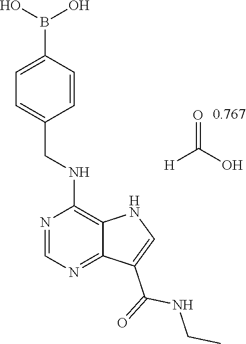 [SMILES: CCNC(=O)c1c[nH]c2c(NCc3ccc(B(O)O)cc3)ncnc12 ; O=CO]

### Step 1: 4-chloro-N-ethyl-5H-pyrrolo[3,2-d]pyrimidine-7-carboxamide

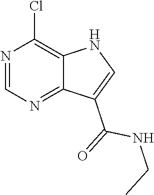

The title compound was synthesized by the same method as described in example 8, step 4 except 4-chloro-5H-pyrrolo[3,2-d]pyrimidine-7-carboxylic acid (150 mg, 0.76 mmol) and ethylamine (455 mg, 30% in EtOH) were used. The title compound (80 mg, 47%) was obtained as a white solid. MS (ESI, pos. ion) m/z: 225.1 (M+1).

### Step 2: 4-([[7-(ethylcarbamoyl)-5H-pyrrolo[3,2-d]pyrimidin-4-yl]amino]methyl)-phenylboronic acid; formic acid

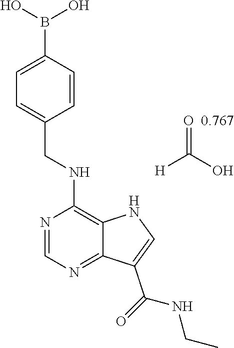 [SMILES: CCNC(=O)c1c[nH]c2c(NCc3ccc(B(O)O)cc3)ncnc12 ; O=CO]

The title compound was synthesized by the same method as described in example 9, step 3 except 4-chloro-N-ethyl-5H-pyrrolo[3,2-d]pyrimidine-7-carboxamide (75 mg, 0.334 mmol) was used. The crude product was purified by prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 4% B to 15% B in 8 min, 15% B; Wave Length: 254; 220 nm;) to afford the title compound (72.3 mg, 55%) as a white solid. MS (ESI, pos. ion) m/z: 340.0 (M+1). ¹H NMR (300 MHz, DMSO-d₆, ppm) δ 12.76 (s, 1H), 11.43 (s, 1H), 8.50 (t, J=5.7 Hz, 1H), 8.34 (s, 1H), 8.14 (s, 1H), 8.01 (d, J=7.8 Hz, 3H), 7.78 (d, J=7.9 Hz, 3H), 7.40-7.31 (m, 2H), 4.76 (d, J=5.5 Hz, 2H), 3.45-3.32 (m, 2H), 1.16 (t, J=7.2 Hz, 3H).

### Example 12

### Synthesis of 4-([[7-(isopropylcarbamoyl)-5H-pyrrolo[3,2-d]pyrimidin-4-yl]amino]methyl)-phenylboronic acid

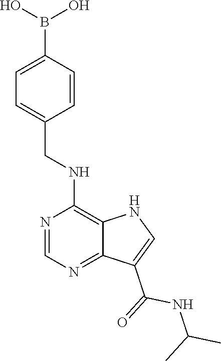 [SMILES: CC(C)NC(=O)c1c[nH]c2c(NCc3ccc(B(O)O)cc3)ncnc12]

### Step 1: 4-chloro-N-isopropyl-5H-pyrrolo[3,2-d]pyrimidine-7-carboxamide

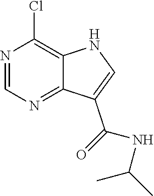 [SMILES: CC(C)NC(=O)c1c[nH]c2c(Cl)ncnc12]

Into a solution of 4-chloro-5H-pyrrolo[3,2-d]pyrimidine-7-carboxylic acid (85 mg, 0.430 mmol, 1.00 equiv) in toluene (5 mL) was added SOCl₂ (3.00 mL) and DMF (0.10 mL). The resulting mixture was stirred for 3 hours at 80° C. After cooling down to room temperature, the reaction mixture concentrated under reduced pressure to afford 4-chloro-5H-pyrrolo[3,2-d]pyrimidine-7-carbonyl chloride (100 mg, crude). A solution of 4-chloro-5H-pyrrolo[3,2-d]pyrimidine-7-carbonyl chloride (100 mg, crude) in toluene (5.00 mL) was treated with TEA (131 mg, 1.291 mmol, 3.00 equiv) and isopropylamine (51 mg, 0.860 mmol, 2.00 equiv) at 0° C. under nitrogen atmosphere. After stirring for 2 h at room temperature, the reaction was quenched with water. The resulting mixture was concentrated under reduced pressure and purified by CombiFlash with the following conditions (MeOH in water, 5% to 30% gradient in 20 min; detector, UV 254/220 nm) to afford the title compound (25 mg, 23%) as a white solid. MS (ESI, pos. ion) m/z: 239.20 (M+1).

### Step 2: 4-([5-methoxypyrrolo[3,2-b]pyridin-1-yl]methyl)phenylboronic acid

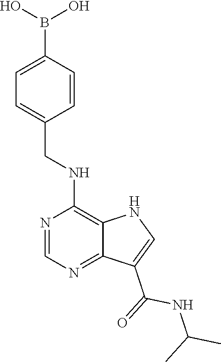 [SMILES: CC(C)NC(=O)c1c[nH]c2c(NCc3ccc(B(O)O)cc3)ncnc12]

The title compound was synthesized by the same method as described in example 6, step 3 except 4-chloro-N-isopropyl-5H-pyrrolo[3,2-d]pyrimidine-7-carboxamide (25 mg, 0.105 mmol) was used. The crude product was purified by prep-HPLC with the following conditions (Column: XSelect CSH Fluoro Phenyl, 30 mm×150 mm, Sum; Mobile Phase A: Water (0.1% FA), Mobile Phase B: ACN; Flow rate: 60 mL/min; Gradient: 3 B to 15 B in 7 min; 254; 220 nm) to afford the title compound (5.7 mg, 15%) as a white solid. MS (ESI, pos. ion) m/z: 354.3 (M+1). ¹H NMR (300 MHz, DMSO/-d₆/D₂O, ppm) δ 8.50 (s, 1H), 8.30 (s, 1H), 7.76 (d, J=7.7 Hz, 2H), 7.36 (d, J=7.7 Hz, 2H), 4.87 (s, 2H), 4.20-3.97 (m, 1H), 1.18 (d, J=6.6 Hz, 6H).

### Example 13

### Synthesis of 4-[([7-[(2-hydroxyethyl)carbamoyl]-5H-pyrrolo[3,2-d]pyrimidin-4-yl]amino)-methyl]phenylboronic acid

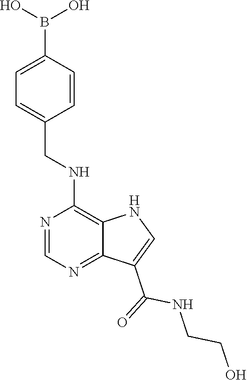 [SMILES: O=C(NCCO)c1c[nH]c2c(NCc3ccc(B(O)O)cc3)ncnc12]

### Step 1: 4-chloro-N-(2-hydroxyethyl)-5H-pyrrolo[3,2-d]pyrimidine-7-carboxamide

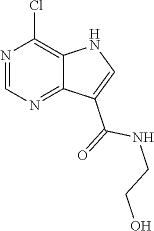 [SMILES: O=C(NCCO)c1c[nH]c2c(Cl)ncnc12]

The title compound was synthesized by the same method as described in example 12, step 1 except 4-chloro-5H-pyrrolo[3,2-d]pyrimidine-7-carboxylic acid (100 mg, 0.5 mmol) and ethanolamine (62 mg, 1.018 mmol) were used. The title compound (30 mg, 22%) was obtained as a white solid. MS (ESI, pos. ion) m/z: 241.0 (M+1).

### Step 2: 4-[([7-[(2-hydroxyethyl)carbamoyl]-5H-pyrrolo[3,2-d]pyrimidin-4-yl]amino)methyl]-phenylboronic acid

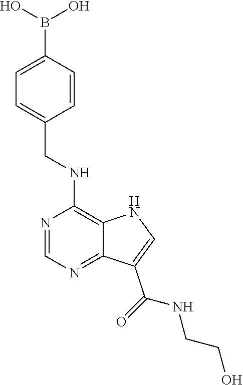 [SMILES: O=C(NCCO)c1c[nH]c2c(NCc3ccc(B(O)O)cc3)ncnc12]

The title compound was synthesized by the same method as described in example 9, step 3 except 4-chloro-N-(2-hydroxyethyl)-5H-pyrrolo[3,2-d]pyrimidine-7-carboxamide (30 mg, 0.125 mmol) was used. The crude product was purified by prep-HPLC with the following conditions (Column: XSelect CSH Fluoro Phenyl, 30 mm×150 mm, Sum; Mobile Phase A: Water (0.1% FA), Mobile Phase B: ACN; Flow rate: 60 mL/min; Gradient: 3 B to 15 B in 7 min; 254; 220 nm) to afford the title compound (8.0 mg, 17%) as a white solid. MS (ESI, pos. ion) m/z: 356.1 (M+1). ¹H NMR (300 MHz, DMSO-d₆) δ 11.48 (s, 1H), 8.67 (t, J=5.6 Hz, 1H), 8.34 (s, 1H), 8.16 (s, 1H), 8.08-7.97 (m, 3H), 7.85-7.68 (m, 3H), 7.35 (d, J=7.9 Hz, 2H), 4.76 (d, J=5.5 Hz, 2H), 3.54 (t, J=5.7 Hz, 2H), 3.43 (t, J=5.5 Hz, 2H).

### Example 14

### Synthesis of 4-[([7-acetamido-5H-pyrrolo[3,2-d]pyrimidin-4-yl]amino)methyl]phenylboronic acid

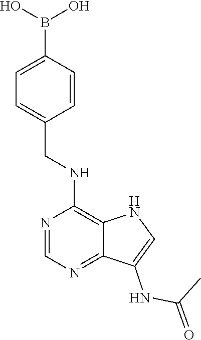 [SMILES: CC(=O)Nc1c[nH]c2c(NCc3ccc(B(O)O)cc3)ncnc12]

### Step 1: 7-nitro-3H,5H-pyrrolo[3,2-d]pyrimidin-4-one

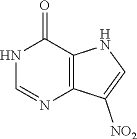 [SMILES: O=c1[nH]cnc2c([N+](=O)[O-])c[nH]c12]

Into a 100-mL round-bottom flask, was placed H₂SO₄ (10 mL, 187.61 mmol, 12.68 equiv). The solution was stirred for 30 min at 0° C. Then 3H,5H-pyrrolo[3,2-d]pyrimidin-4-one (2.00 g, 14.80 mmol, 1.00 equiv) was added and stirred for 30 min at 0° C. Then HNO₃ (5 mL, 111.49 mmol, 7.5 equiv) was added and stirred for 30 min at 0° C. The resulting mixture was allowed to warm to room temperature and stirred for 2 h. The reaction mixture was basified to pH 7 with saturated aqueous sodium bicarbonate. The precipitate was collected by filtration, washed with water and dried in vacuo to afford the title compound (2.10 g, 79%) as a brown solid.

### Step 2: 4-chloro-7-nitro-5H-pyrrolo[3,2-d]pyrimidine

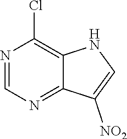 [SMILES: O=[N+]([O-])c1c[nH]c2c(Cl)ncnc12]

To a stirred solution of 7-nitro-3H,5H-pyrrolo[3,2-d]pyrimidin-4-one (2.14 g, 11.88 mmol, 1.00 equiv) in ACN (50 mL) was added POCl₃ (12 mL) at room temperature. After stirring for 12 h at 80° C. the reaction mixture was cooled to room temperature and concentrated under reduced pressure. The residue was purified by CombiFlash with the following conditions, ACN in water, 5% to 40% gradient in 13 min; detector, UV 254/220 nm.) to give the title compound (1.72 g, 73%) as a yellow solid.

### Step 3: 4-[([7-nitro-5H-pyrrolo[3,2-d]pyrimidin-4-yl]amino)methyl]phenylboronic acid

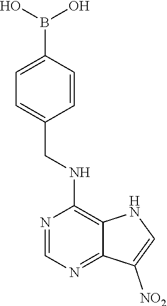 [SMILES: O=[N+]([O-])c1c[nH]c2c(NCc3ccc(B(O)O)cc3)ncnc12]

The title compound was synthesized by the same method as described in example 10, step 2 except 4-chloro-7-nitro-5H-pyrrolo[3,2-d]pyrimidine (500 mg, 2.52 mmol) was used. The title compound (380 mg, 44%) was obtained as a yellow solid.

### Step 4: 4-[([7-amino-5H-pyrrolo[3,2-d]pyrimidin-4-yl]amino)methyl]phenylboronic acid

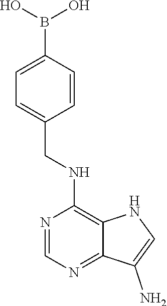 [SMILES: Nc1c[nH]c2c(NCc3ccc(B(O)O)cc3)ncnc12]

To a solution of 4-[([7-nitro-5H-pyrrolo[3,2-d]pyrimidin-4-yl]amino)methyl]-phenylboronic acid (1 equiv.) in methanol was added Pd/C and the reaction mixture was stirred under hydrogen atmosphere for 3 h. The reaction mixture was filtered through Celite and the filter cake was washed with methanol. The filtrate was concentrated to give the title compound was obtained as a brown solid.

### Step 5: 4-[([7-acetamido-5H-pyrrolo[3,2-d]pyrimidin-4-yl]amino)methyl]phenylboronic acid

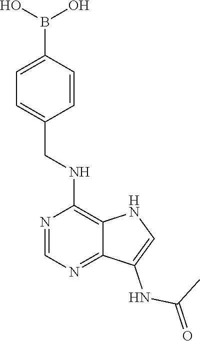 [SMILES: CC(=O)Nc1c[nH]c2c(NCc3ccc(B(O)O)cc3)ncnc12]

To a stirred solution of 4-[([7-amino-5H-pyrrolo[3,2-d]pyrimidin-4-yl]amino)methyl]-phenylboronic acid (20 mg, 0.07 mmol, 1.00 equiv) in (Ac)₂0 (1.50 mL) was added DMAP (14 mg, 0.11 mmol, 1.60 equiv) at 0° C. The resulting mixture was stirred for 0.5 h at 0° C. After concentration under reduced pressure, the crude product was purified by prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 2% B to 10% B in 8 min, 10% B; Wave Length: 254/220 nm) to afford the title compound (15.5 mg, 67%) as a white solid. MS (ESI, pos. ion) m/z: 326.3 (M+1). ¹H NMR (300 MHz, DMSO-d₆, ppm) δ 10.64 (s, 1H), 9.95 (s, 1H), 8.18 (d, J=8.3 Hz, 2H), 8.06 (s, 1H), 7.91 (d, J=2.4 Hz, 1H), 7.84-7.73 (m, 2H), 7.57 (t, J=5.6 Hz, 1H), 7.35 (d, J=7.8 Hz, 2H), 4.74 (d, J=5.6 Hz, 2H), 2.08 (s, 3H)

### Example 15

### Synthesis of 4-[([7-acetamidothieno[3,2-d]pyrimidin-4-yl]amino)methyl]phenylboronic acid

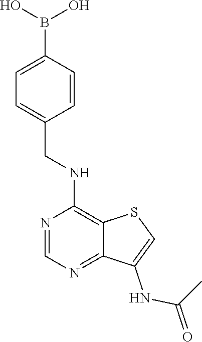 [SMILES: CC(=O)Nc1csc2c(NCc3ccc(B(O)O)cc3)ncnc12]

### Step 1: 7-nitro-3H-thieno[3,2-d]pyrimidin-4-one

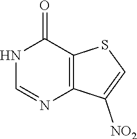 [SMILES: O=c1[nH]cnc2c([N+](=O)[O-])csc12]

The title compound was synthesized by the same method as described in example 14, step 1 except 3H-thieno[3,2-d]pyrimidin-4-one (2.00 g, 13.143 mmol) and iethyl 4-(aminomethyl)-phenylphosphonate (378 mg, 1.55 mmol) were used. The title compound (1.5 g, 56%) was obtained as a white solid.

### Step 2: 4-chloro-7-nitrothieno[3,2-d]pyrimidine

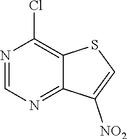 [SMILES: O=[N+]([O-])c1csc2c(Cl)ncnc12]

The title compound was synthesized by the same method as described in example 14, step 2 except 7-nitro-3H-thieno[3,2-d]pyrimidin-4-one (1.00 g, 5.072 mmol was used. The title compound (440 mg, 40%) as a white solid was obtained.

### Step 3: 4-[([7-nitrothieno[3,2-d]pyrimidin-4-yl]amino)methyl]phenylboronic acid

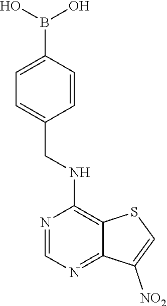 [SMILES: O=[N+]([O-])c1csc2c(NCc3ccc(B(O)O)cc3)ncnc12]

The title compound was synthesized by the same method as described in example 10, step 2 except 4-chloro-7-nitrothieno[3,2-d]pyrimidine (440 mg, 2.041 mmol) and 4-(aminomethyl)-phenylboronic acid hydrochloride (1.1 g, 6.122 mmol) were used. The title compound (250 mg, 37%) was obtained as a yellow solid. MS (ESI, pos. ion) m/z: 331.0 (M+1).

### Step 4: 4-[([7-aminothieno[3,2-d]pyrimidin-4-yl]amino)methyl]phenylboronic acid

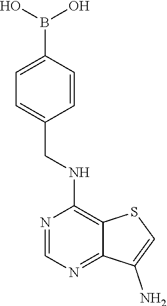 [SMILES: Nc1csc2c(NCc3ccc(B(O)O)cc3)ncnc12]

To a stirred mixture of 4-[([7-nitrothieno[3,2-d]pyrimidin-4-yl]amino)methyl]-phenylboronic acid (174 mg, 0.527 mmol, 1.00 equiv) in EtOH (3.00 mL) and H₂O (1.00 mL) were added Fe (294 mg, 5.270 mmol, 10.00 equiv) and NH₄Cl (282 mg, 5.270 mmol, 10.00 equiv) at room temperature. After stirring for 12 h at 80° C. the mixture was cooled down to room temperature and filtered through a Celite. The filter cake was washed with EtOH. The filtrate was concentrated under reduced pressure. The residue was purified by column chromatography on silica gel 80 g (eluent: dichloromethane methyl alcohol 100%, 89: 11) to give 100 mg crude product. The crude product was further purified by CombiFlash to afford the title compound (39 mg, 24%) as a white solid. MS (ESI, pos. ion) m/z: 301.2 (M+1).

### Step 5: 4-[([7-acetamidothieno[3,2-d]pyrimidin-4-yl]amino)methyl]phenylboronic acid

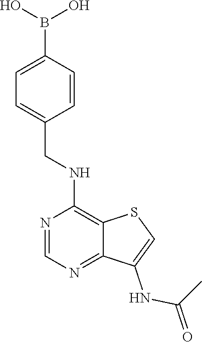 [SMILES: CC(=O)Nc1csc2c(NCc3ccc(B(O)O)cc3)ncnc12]

The title compound was synthesized by the same method as described in example 14, step 5 except 4-[([7-aminothieno[3,2-d]pyrimidin-4-yl]amino)methyl]phenylboronic acid (35.00 mg, 0.117 mmol) was used. The crude product was purified by prep-HPLC with the following conditions (Column: Xselect CSH OBD Column 30*150 mm Sum, n; Mobile Phase A: Water (10 mmolL/L NH₄HCO₃+0.1% NH3·H₂O), Mobile Phase B: ACN; Flow rate: 60 mL/min; Gradient: 5% B to 30% B in 8 min, 30% B; Wave Length: 254/220 nm;) to afford the title compound (16 mg, 70%) as a white solid. MS (ESI, pos. ion) m/z: 343.2 (M+1). ¹H NMR (300 MHz, DMSO-d₆, ppm) δ 10.20 (s, 1H), 8.51 (d, J=14.1 Hz, 2H), 8.22 (s, 1H), 7.99 (s, 2H), 7.73 (d, J=7.7 Hz, 2H), 7.30 (d, J=7.7 Hz, 2H), 4.75 (d, J=6.0 Hz, 2H), 2.17 (s, 3H).

### Example 16

### Synthesis of 4-[([1,5-dimethylpyrazolo[4,3-d]pyrimidin-7-yl]amino)methyl]phenylboronic acid

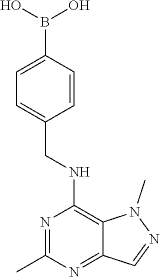 [SMILES: Cc1nc(NCc2ccc(B(O)O)cc2)c2c(cnn2C)n1]

### Step 1: methyl 4-amino-2-methylpyrazole-3-carboxylate

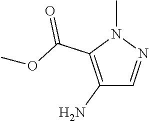

To a solution of methyl 2-methyl-4-nitropyrazole-3-carboxylate (2.00 g, 1.00 equiv) in MeOH (30.00 mL) was added Pd/C (115 mg, 1.080 mmol, 0.10 equiv) at room temperature. After stirring for overnight at room temperature under hydrogen atmosphere using a hydrogen balloon, the reaction mixture was filtered through a Celite pad. The filtrate was concentrated under reduced pressure to give the title compound (1.3 g, 77%) as a grew solid.

### Step 2: methyl 4-ethanimidamido-2-methylpyrazole-3-carboxylate

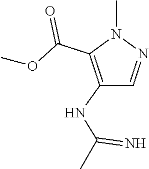

A solution of methyl 4-amino-2-methylpyrazole-3-carboxylate (660 mg, 4.254 mmol, 1.00 equiv) in ACN (10.00 mL) was treated with HCl (gas, 4 M in 1,4-dioxane, 0.78 mL) at 0° C. under nitrogen atmosphere. After stirring for 18 h at 50° C., the resulting mixture was concentrated under reduced pressure. The resulting mixture was diluted with water (30 mL) and neutralized to pH 7.0 with NaOH (2 M). The precipitated solid was collected by filtration and dried in vacuo to give the title compound (500 mg, 60%) as an off-white solid.

### Step 3: 1,5-dimethyl-6H-pyrazolo[4,3-d]pyrimidin-7-one

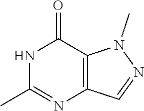

To a solution of methyl 4-ethanimidamido-2-methylpyrazole-3-carboxylate (400 mg, 2.039 mmol, 1.00 equiv) in EtOH (10 mL) was added NaOH (1.4 mL, 8.156 mmol, 6 M in water) at room temperature. The mixture was stirred for 4 h at 90° C. After cooling to room temperature, the resulting mixture was basified to pH 4-5 with HCl (3 M). The precipitated solid was collected by filtration and dried in vacuo to give the title compound (220 mg, 66%) as an off-white solid.

### Step 4: 7-chloro-1,5-dimethylpyrazolo[4,3-d]pyrimidine

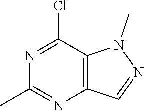

To a solution of 1,5-dimethyl-6H-pyrazolo[4,3-d]pyrimidin-7-one (220 mg, 1.340 mmol, 1.00 equiv) in SOCl₂ (5 mL) was added DMF (0.10 mL) at room temperature. After stirring for 2 h at 80° C., the reaction mixture was cooled to room temperature and concentrated under reduced pressure. The residue was purified CombiFlash with the following conditions, ACN in water, 10% to 50% gradient in 30 min; detector, UV 254/220 nm.) to afford the title compound (80 mg, 33%) as an off-white solid.

### Step 5: 4-[([1,5-dimethylpyrazolo[4,3-d]pyrimidin-7-yl]amino)methyl]phenylboronic acid

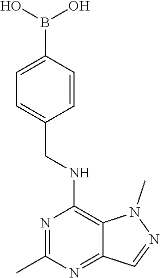 [SMILES: Cc1nc(NCc2ccc(B(O)O)cc2)c2c(cnn2C)n1]

To a solution of 7-chloro-1,5-dimethylpyrazolo[4,3-d]pyrimidine (70 mg, 0.383 mmol, 1.00 equiv) in DMSO (5 mL) were added TEA (116 mg, 1.149 mmol, 3.00 equiv) and 4-(aminomethyl)phenylboronic acid hydrochloride (144 mg, 0.767 mmol, 2.00 equiv) at room temperature. After stirring for 12 h at 80° C., the reaction mixture was cooled to room temperature and concentrated under reduced pressure. The crude product was purified by prep-HPLC with the following conditions (Column: XBridge Prep OBD C18 Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 11% B to 25% B in 7 min, 25% B; Wave Length: 254/220 nm) to afford the title compound (53.0 mg, 45%) as a white solid. MS (ESI, pos. ion) m/z: 298.2 (M+1). ¹H NMR (300 MHz, DMSO-d₆) δ 7.99 (s, 1H), 7.84 (d, J=4.1 Hz, 1H), 7.75 (dd, J=7.2, 5.6 Hz, 3H), 7.37 (d, J=7.8 Hz, 2H), 4.77 (d, J=5.8 Hz, 2H), 4.26 (s, 3H), 2.37 (s, 3H).

### Example 17

### Synthesis of 4-[([5-isopropyl-1-methylpyrazolo[4,3-d]pyrimidin-7-yl]amino)methyl]-phenylboronic acid

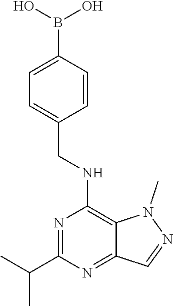 [SMILES: CC(C)c1nc(NCc2ccc(B(O)O)cc2)c2c(cnn2C)n1]

### Step 1: methyl 2-methyl-4-(2-methylpropanimidamido)pyrazole-3-carboxylate

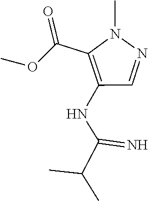 [SMILES: COC(=O)c1c(NC(=N)C(C)C)cnn1C]

The title compound was synthesized by the same method as described in example 16, step 2 except methyl 4-amino-2-methylpyrazole-3-carboxylate (1.20 g, 7.734 mmol) and isobutyronitrile (15 mL) were used. The title compound (1.3 g, 75%) was obtained as an off-white solid. MS (ESI, pos. ion) m/z: 225.2 (M+1).

### Step 2: 5-isopropyl-1-methyl-6H-pyrazolo[4,3-d]pyrimidin-7-one

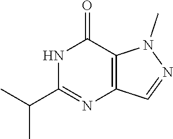

The title compound was synthesized by the same method as described in example 16, step 3 except methyl 4-amino-2-methylpyrazole-3-carboxylate (1.20 g, 7.734 mmol) was used. The title compound (0.7 g, 68%) was obtained as an off-white solid.

### Step 3: 7-chloro-5-isopropyl-1-methylpyrazolo[4,3-d]pyrimidine

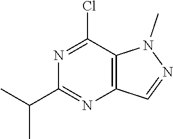

The title compound was synthesized by the same method as described in example 16, step 4 except 5-isopropyl-1-methyl-6H-pyrazolo[4,3-d]pyrimidin-7-one (200 mg, 1.040 mmol) was used. The title compound (85 mg, 39%) was obtained as an off-white solid.

### Step 4: 4-[([5-Isopropyl-1-methylpyrazolo[4,3-d]pyrimidin-7-yl]amino)methyl]-phenylboronic acid

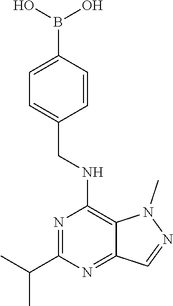 [SMILES: CC(C)c1nc(NCc2ccc(B(O)O)cc2)c2c(cnn2C)n1]

The title compound was synthesized by the same method as described in example 16, step 4 except 7-chloro-5-isopropyl-1-methylpyrazolo[4,3-d]pyrimidine (85 mg, 0.403 mmol) was used. The crude product was purified by prep-HPLC with the following conditions (Column: XBridge Shield RP18 OBD Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 26% B to 36% B in 8 min, 36% B; Wave Length: 254; 220 nm) to afford the title compound (20.8 mg, 16%) as a white solid. MS (ESI, pos. ion) m/z: 326.3 (M+1). ¹H NMR (300 MHz, DMSO-d₆) δ 7.97 (s, 1H), 7.88 (s, 1H), 7.82 (t, J=5.9 Hz, 1H), 7.74 (d, J=7.6 Hz, 2H), 7.50-7.31 (m, 2H), 4.76 (d, J=5.9 Hz, 2H), 4.27 (s, 3H), 2.89 (p, J=6.9 Hz, 1H), 1.16 (d, J=6.9 Hz, 6H).

### Example 18

### Synthesis of 4-([[3-(methylcarbamoyl)-1H-pyrazolo[4,3-d]pyrimidin-7-yl]amino]methyl)-phenylboronic acid

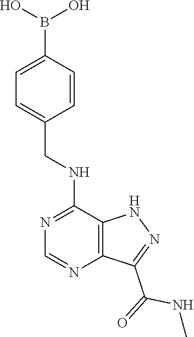 [SMILES: CNC(=O)c1n[nH]c2c(NCc3ccc(B(O)O)cc3)ncnc12]

### Step 1: 1-tert-butyl 3,5-dimethyl 4-nitropyrazole-1,3,5-tricarboxylate

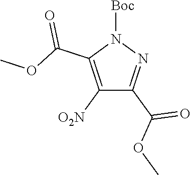 [SMILES: COC(=O)c1nn(C(=O)OC(C)(C)C)c(C(=O)OC)c1[N+](=O)[O-]]

To a solution of 3,5-dimethyl 4-nitro-1H-pyrazole-3,5-dicarboxylate (3.00 g, 13.09 mmol, 1.00 equiv) in THE (20 mL) were added TEA (3.97 g, 39.23 mmol, 3.00 equiv) and (Boc)₂O (6.86 g, 31.43 mmol, 2.40 equiv) at room temperature. After stirring overnight at 40° C., the reaction mixture was cooled to room temperature and concentrated under reduced pressure. The residue was purified by column chromatography on silica gel 120 g (eluent: petroleum ether ethyl acetate 100%, 70: 30) to give the title compound (2.5 g, 54%) as a colorless oil.

### Step 2: 1-tert-butyl 3,5-dimethyl 4-aminopyrazole-1,3,5-tricarboxylate

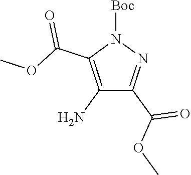 [SMILES: COC(=O)c1nn(C(=O)OC(C)(C)C)c(C(=O)OC)c1N]

The title compound was synthesized by the same method as described in example 14, step 4 except 1-tert-butyl 3,5-dimethyl 4-nitropyrazole-1,3,5-tricarboxylate (1.70 g, 5.16 mmol) was used. The title compound (1.43 g, 86%) was obtained as an off-white solid.

### Step 3: 1-tert-butyl 3-methyl 7-oxo-6H-pyrazolo[4,3-d]pyrimidine-1,3-dicarboxylate

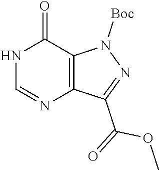 [SMILES: COC(=O)c1nn(C(=O)OC(C)(C)C)c2c(=O)[nH]cnc12]

To a stirred solution of 1-tert-butyl 3,5-dimethyl 4-aminopyrazole-1,3,5-tricarboxylate (2.00 g, 6.68 mmol, 1.00 equiv) in MeOH (40 mL) was added formamidine acetate (0.83 g, 8.02 mmol, 1.20 equiv) at room temperature. After stirring for 12 h at 105° C., the reaction mixture was cooled to room temperature and concentrated under reduced pressure. The residue was purified by CombiFlash with the following conditions, MeOH in water, 10% to 100% gradient in 30 min; detector, UV 254/220 nm.) to afford the title compound (1.2 g, 61%) as an off-white solid.

### Step 4: 1-tert-butyl 3-methyl 7-chloropyrazolo[4,3-d]pyrimidine-1,3-dicarboxylate

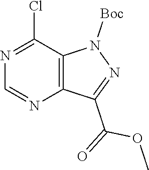 [SMILES: COC(=O)c1nn(C(=O)OC(C)(C)C)c2c(Cl)ncnc12]

To a solution of 1-tert-butyl 3-methyl 7-oxo-6H-pyrazolo[4,3-d]pyrimidine-1,3-dicarboxylate (1.20 g, 4.08 mmol, 1.00 equiv) in SO₂Cl₂ (20 mL) was added DMF (0.10 mL) at room temperature. After stirring for 2 h at 80° C., the reaction mixture was cooled to room temperature and concentrated under reduced pressure. The residue was purified CombiFlash with the following conditions, ACN in water, 10% to 50% gradient in 30 min; detector, UV 254/220 nm.) to afford the title compound (565 mg, 44%) as an off-white solid.

### Step 5: 4-([[1-(tert-butoxycarbonyl)-3-(methoxycarbonyl)pyrazolo[4,3-d]pyrimidin-7-yl]amino]-methyl)phenylboronic acid

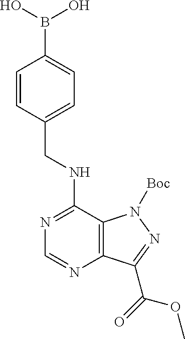 [SMILES: COC(=O)c1nn(C(=O)OC(C)(C)C)c2c(NCc3ccc(B(O)O)cc3)ncnc12]

The title compound was synthesized by the same method as described in example 10, step 2 except 1-tert-butyl 3-methyl 7-chloropyrazolo[4,3-d]pyrimidine-1,3-dicarboxylate (140 mg, 0.45 mmol) and 4-(aminomethyl)phenylboronic acid hydrochloride (126 mg, 0.50 mmol) were used. The title compound (140 mg, 73%) was obtained as an light brown solid.

### Step 6: 4-([[1-(tert-butoxycarbonyl)-3-(methylcarbamoyl)pyrazolo[4,3-d]pyrimidin-7-yl]-amino]-methyl)phenylboronic acid

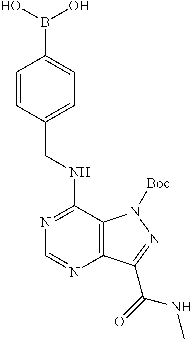 [SMILES: CNC(=O)c1nn(C(=O)OC(C)(C)C)c2c(NCc3ccc(B(O)O)cc3)ncnc12]

The title compound was synthesized by the same method as described in example 3, step 1 except 4-([[1-(tert-butoxycarbonyl)-3-(methoxycarbonyl)pyrazolo[4,3-d]pyrimidin-7-yl]amino]methyl)phenylboronic acid (100 mg, 0.23 mmol) was used. The title compound (80 mg, 74%) was obtained as a white solid.

### Step 7: 4-([[3-(methylcarbamoyl)-1H-pyrazolo[4,3-d]pyrimidin-7-yl]amino]methyl)-phenyl-boronic acid

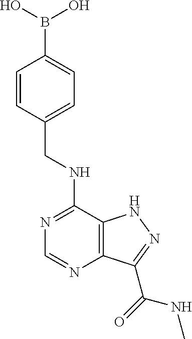 [SMILES: CNC(=O)c1n[nH]c2c(NCc3ccc(B(O)O)cc3)ncnc12]

A solution of 4-([[1-(tert-butoxycarbonyl)-3-(methylcarbamoyl)pyrazolo[4,3-d]pyrimidin-7-yl]amino]-methyl)phenylboronic acid (I equiv.) was stirred in 4 mL HCl (gas, 4 M in 1,4-dioxane) was stirred for 4 h at 50° C. After cooling, the reaction mixture to room temperature, the reaction mixture was concentrated under vacuum. The crude product was purified by prep-HPLC with the following conditions (Column: XBridge Prep OBD C18 Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 4% B to 15% B in 7 min, 15% B; Wave Length: 254/220 nm) to give the title compound (14.6 mg, 32%) as a white solid. MS (ESI, pos. ion) m/z: 327.3 (M+1). ¹H NMR (300 MHz, DMSO-d₆, ppm) δ 10.71 (s, 1H), 8.77 (s, 1H), 8.62 (s, 1H), 7.78 (d, J=7.6 Hz, 2H), 7.39 (d, J=7.6 Hz, 2H), 4.94 (d, J=5.7 Hz, 2H), 2.87 (d, J=4.6 Hz, 3H).

### Example 19

### Synthesis of 4-([[7-(2-methylpropanamido)thieno[3,2-d]pyrimidin-4-yl]amino]methyl)-phenylboronic acid

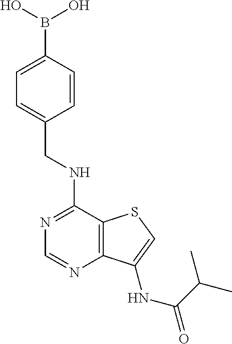 [SMILES: CC(C)C(=O)Nc1csc2c(NCc3ccc(B(O)O)cc3)ncnc12]

### Step 1: 7-nitrothieno[3,2-d]pyrimidin-4-ol

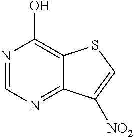 [SMILES: O=[N+]([O-])c1csc2c(O)ncnc12]

The title compound was synthesized by the same method as described in example 14, step 1 except 4-chlorothieno[3,2-d]pyrimidine (5 g, 29.31 mmol) was used. The title compound (5 g, 83%) was obtained as a white solid.

### Step 2: 4-chloro-7-nitrothieno[3,2-d]pyrimidine

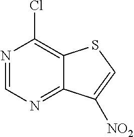 [SMILES: O=[N+]([O-])c1csc2c(Cl)ncnc12]

To a stirred solution of 7-nitrothieno[3,2-d]pyrimidin-4-ol (4.60 g, 23.33 mmol, 1.00 equiv) in thionyl chloride (120 mL) was added DMF (0.50 mL) at room temperature. After stirring for 2 h at 80° C., the reaction mixture was cooled to room temperature and concentrated under reduced pressure. The residue was purified by Combiflash with the following conditions, ACN in water, 10% to 40% gradient in 20 min; detector, UV 254/220 nm.) to give the title compound (3.26 g, 64%) as a white solid.

### Step 3: 4-chlorothieno[3,2-d]pyrimidin-7-amine

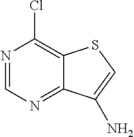 [SMILES: Nc1csc2c(Cl)ncnc12]

To a stirred solution of 4-chloro-7-nitrothieno[3,2-d]pyrimidine (3.26 g, 15.12 mmol, 1.00 equiv) in EtOH (180 mL) and H₂O (30 mL) were added Fe (8.44 g, 0.15 mmol, 10.00 equiv) and NH₄Cl (8.09 g, 0.15 mmol, 10.00 equiv) at room temperature under e nitrogen. The resulting mixture was stirred for 2 h at 80° C. After cooling down to room temperature, the reaction mixture was filtered through a Celite and the filter cake was washed with EtOH. The filtrate was concentrated under reduced pressure. The residue was purified by column chromatography on silica gel 120 g (eluent: petroleum ether ethyl acetate 100%, 64: 36) to give the title compound (1.8 g, 63%) as a yellow solid.

### Step 4: N-[4-chlorothieno[3,2-d]pyrimidin-7-yl]-2-methylpropanamide

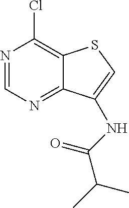 [SMILES: CC(C)C(=O)Nc1csc2c(Cl)ncnc12]

To a solution of 4-chlorothieno[3,2-d]pyrimidin-7-amine (1 equiv) in DCM was added DIEA (2 equiv) and isobutyryl chloride (1.62 mmol) at 0° C. The mixture was concentrated after stirring it for stirred for 1 h. After purification, the title compound was obtained as a white solid. (M+1).

### Step 5: 4-([[7-(2-methylpropanamido)thieno[3,2-d]pyrimidin-4-yl]amino]methyl)-phenylboronic acid

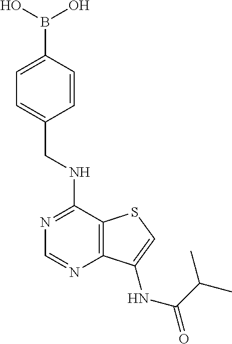 [SMILES: CC(C)C(=O)Nc1csc2c(NCc3ccc(B(O)O)cc3)ncnc12]

The title compound was synthesized by the same method as described in example 10, step 2 except N-[4-chlorothieno[3,2-d]pyrimidin-7-yl]-2-methylpropanamide (75 mg, 0.29 mmol) and 4-(aminomethyl)phenylboronic acid hydrochloride (77 mg, 0.41 mmol) were used. The crude product was purified by prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 22% B to 35% B in 8 min, 35% B; Wave Length: 254; 220 nm;) to give 4-([[7-(2-methylpropanamido)thieno[3,2-d]pyrimidin-4-yl]amino]methyl)phenylboronic acid (89.9 mg, 82%) as a white solid. MS (ESI, pos. ion) m/z: 371.2 (M+1). ¹H NMR (300 MHz, DMSO-d₆, ppm) δ 10.04 (s, 1H), 8.52 (d, J=13.9 Hz, 2H), 8.25 (s, 1H), 7.99 (s, 2H), 7.74 (d, J=7.8 Hz, 2H), 7.30 (d, J=7.8 Hz, 2H), 4.75 (d, J=5.9 Hz, 2H), 2.98 (p, J=6.7 Hz, 1H), 1.10 (d, J=6.8 Hz, 6H).

### Example 20

### Synthesis of 4-[([7-propanamidothieno[3,2-d]pyrimidin-4-yl]amino)methyl]-phenylboronic acid

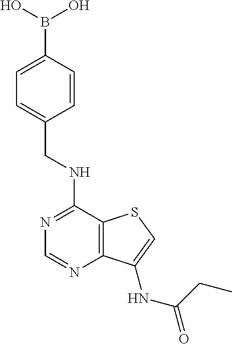 [SMILES: CCC(=O)Nc1csc2c(NCc3ccc(B(O)O)cc3)ncnc12]

### Step 1: N-[4-chlorothieno[3,2-d]pyrimidin-7-yl]propanamide

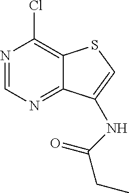

The title compound was synthesized by the same method as described in example 19, step 4 except propanoyl chloride (150 mg, 1.62 mmol) was used. The title compound (189 mg 96%) was obtained as a white solid.

### Step 2: 4-[([7-propanamidothieno[3,2-d]pyrimidin-4-yl]amino)methyl]phenylboronic acid

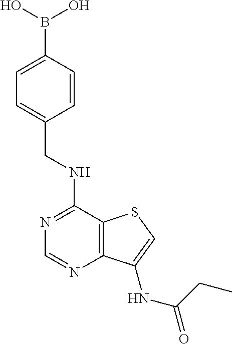 [SMILES: CCC(=O)Nc1csc2c(NCc3ccc(B(O)O)cc3)ncnc12]

The title compound was synthesized by the same method as described in example 10, step 2 except N-[4-chlorothieno[3,2-d]pyrimidin-7-yl]propanamide (80 mg, 0.33 mmol) and 4-(aminomethyl)phenylboronic acid hydrochloride (87 mg, 0.46 mmol) were used. The crude product was purified by prep-HPLC with the following conditions (Column: Sunfire prep C18 column, 30*150 mm, 5 m; Mobile Phase A: Water (0.1% FA), Mobile Phase B: ACN; Flow rate: 60 mL/min; Gradient: 17% B to 35% B in 8 min, 35% B; Wave Length: 254/220 nm;) to give the title compound (58.2 mg, 49%) as a white solid. MS (ESI, pos. ion) m/z: 357.0 (M+1). ¹H NMR (300 MHz, DMSO-d₆ ppm) δ 10.09 (s, 1H), 8.51 (d, J=14.2 Hz, 2H), 8.24 (s, 1H), 7.98 (s, 2H), 7.78-7.69 (m, 2H), 7.30 (d, J=7.8 Hz, 2H), 4.75 (d, J=5.9 Hz, 2H), 2.57-2.44 (m, 2H), 1.09 (t, J=7.5 Hz, 3H).

### Example 21

### Synthesis of 4-[([7-benzamidothieno[3,2-d]pyrimidin-4-yl]amino)methyl]-phenylboronic acid

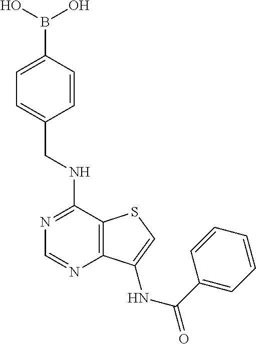 [SMILES: O=C(Nc1csc2c(NCc3ccc(B(O)O)cc3)ncnc12)c1ccccc1]

### Step 1: N-[4-chlorothieno[3,2-d]pyrimidin-7-yl]benzamide

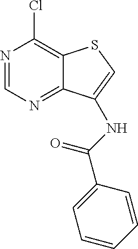 [SMILES: O=C(Nc1csc2c(Cl)ncnc12)c1ccccc1]

The title compound was synthesized by the same method as described in example 19, step 4 except benzoyl chloride (227 mg, 1.616 mmol) was used. The title compound (208 mg, 89%) was obtained as a white solid.

### Step 2: 4-[([7-benzamidothieno[3,2-d]pyrimidin-4-yl]amino)methyl]phenylboronic acid

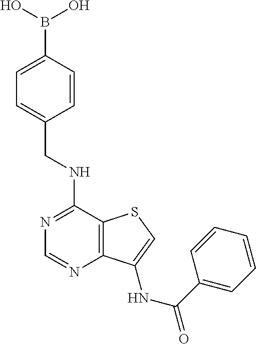 [SMILES: O=C(Nc1csc2c(NCc3ccc(B(O)O)cc3)ncnc12)c1ccccc1]

The title compound was synthesized by the same method as described in example 10, step 2 except N-[4-chlorothieno[3,2-d]pyrimidin-7-yl]benzamide (100 mg, 0.35 mmol) and 4-(aminomethyl)phenylboronic acid hydrochloride (91 mg, 0.48 mmol) were used. The crude product was purified by prep-HPLC with the following conditions (Column: XSelect CSH Fluoro Phenyl, 30*150 mm, 5 μm; Mobile Phase A: Water (0.1% FA), Mobile Phase B: ACN; Flow rate: 60 mL/min; Gradient: 25% B to 50% B in 8 min, 50% B; Wave Length: 254; 220 nm;) to give the title compound (13.5 mg, 9%) as a white solid. MS (ESI, pos. ion) m/z: 404.9 (M+1). ¹H NMR (300 MHz, DMSO-d₆ ppm) δ 9.98 (s, 1H), 8.62 (t, J=6.0 Hz, 1H), 8.52 (s, 1H), 8.34 (s, 1H), 8.07-7.96 (m, 4H), 7.79-7.70 (m, 2H), 7.70-7.51 (m, 3H), 7.32 (d, J=7.7 Hz, 2H), 4.78 (d, J=5.9 Hz, 2H).

### Example 22

### Synthesis of 4-[([7-acetamido-5-methylpyrrolo[3,2-d]pyrimidin-4-yl]amino)methyl]-phenylboronic acid

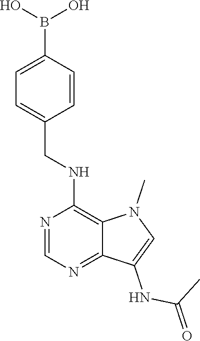 [SMILES: CC(=O)Nc1cn(C)c2c(NCc3ccc(B(O)O)cc3)ncnc12]

### Step 1: 5-methyl-7-nitropyrrolo[3,2-d]pyrimidin-4-ol

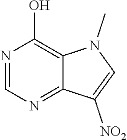

The title compound was synthesized by the same method as described in example 14, step 1 except 4-chloro-5-methylpyrrolo[3,2-d]pyrimidine (800 mg, 4.77 mmol) was used. The title compound (600 mg, 65%) was obtained as a white solid.

### Step 2: 4-chloro-5-methyl-7-nitropyrrolo[3,2-d]pyrimidine

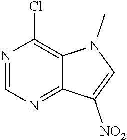

The title compound was synthesized by the same method as described in example 19, step 2 except 5-methyl-7-nitropyrrolo[3,2-d]pyrimidin-4-ol (350 mg, 1.80 mmol) was used. The title compound (130 mg, 34%) was obtained as a white solid.

### Step 3: 4-chloro-5-methylpyrrolo[3,2-d]pyrimidin-7-amine

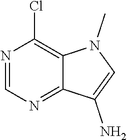

The title compound was synthesized by the same method as described in example 19, step 3 except 4-chloro-5-methyl-7-nitropyrrolo[3,2-d]pyrimidine (130 mg, 0.61 mmol) was used.

The title compound (100 mg, 80%) was obtained as a white solid.

### Step 4: N-[4-chloro-5-methylpyrrolo[3,2-d]pyrimidin-7-yl]acetamide

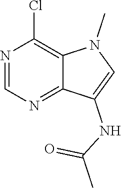

The title compound was synthesized by the same method as described in example 19, step 4 except 4-chloro-5-methylpyrrolo[3,2-d]pyrimidin-7-amine (100 mg, 0.55 mmol) was used.

The title compound (100 mg, 79%) was obtained as a white solid.

### Step 5: 4-[([7-acetamido-5-methylpyrrolo[3,2-d]pyrimidin-4-yl]amino)methyl]phenylboronic acid

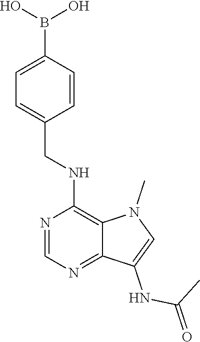 [SMILES: CC(=O)Nc1cn(C)c2c(NCc3ccc(B(O)O)cc3)ncnc12]

The title compound was synthesized by the same method as described in example 10, step 2 except N-[4-chloro-5-methylpyrrolo[3,2-d]pyrimidin-7-yl]acetamide (100 mg, 0.45 mmol) and 4-(aminomethyl)phenylboronic acid hydrochloride (116 mg, 0.62 mmol) were used. The crude product was purified by prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 5% B to 15% B in 8 min, 15% B; Wave Length: 254; 220 nm;) to give 4-[([7-acetamido-5-methylpyrrolo[3,2-d]pyrimidin-4-yl]amino)methyl]phenylboronic acid (42.5 mg, 28%) as a white solid. MS (ESI, pos. ion) m/z: 340.0 (M+1). ¹H NMR (300 MHz, DMSO-d₆ ppm) δ 9.91 (s, 1H), 8.19-8.06 (m, 2H), 7.98 (s, 1H), 7.81 (d, J=5.2 Hz, 1H), 7.73 (dd, J=7.9, 4.0 Hz, 2H), 7.33 (dd, J=7.9, 3.2 Hz, 3H), 4.75 (d, J=5.6 Hz, 2H), 4.04 (d, J=4.6 Hz, 3H), 2.08 (s, 3H).

### Example 23

### Synthesis of 4-[([7-formamidothieno[3,2-d]pyrimidin-4-yl]amino)methyl]phenylboronic acid

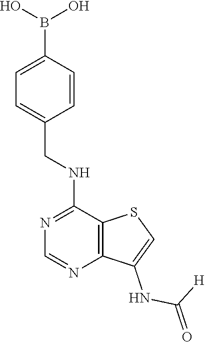 [SMILES: O=CNc1csc2c(NCc3ccc(B(O)O)cc3)ncnc12]

### Step 1: N-[4-chlorothieno[3,2-d]pyrimidin-7-yl]formamide

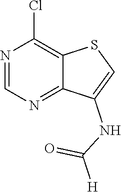 [SMILES: O=CNc1csc2c(Cl)ncnc12]

A solution of 4-chlorothieno[3,2-d]pyrimidin-7-amine (150 mg, 0.81 mmol, 1.00 equiv) in formic acid (5.00 mL) was stirred for 2 h at room temperature. The reaction mixture was concentrated under reduced pressure. The residue was purified by column chromatography on silica gel 40 g (eluent: petroleum ether ethyl acetate 100%, 50: 50) to give the title compound (145 mg, 84%) as a white solid.

### Step 2: 4-[([7-formamidothieno[3,2-d]pyrimidin-4-yl]amino)methyl]phenylboronic acid

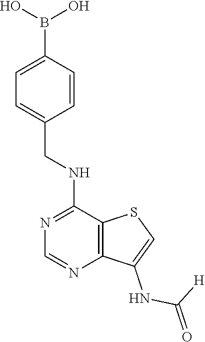 [SMILES: O=CNc1csc2c(NCc3ccc(B(O)O)cc3)ncnc12]

The title compound was synthesized by the same method as described in example 10, step 2 except N-[4-chlorothieno[3,2-d]pyrimidin-7-yl]formamide (120 mg, 0.56 mmol) and 4-(aminomethyl)phenyl-boronic acid hydrochloride (168 mg, 0.90 mmol) were used. The crude product was purified by prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 6% B to 26% B in 7 min, 26% B; Wave Length: 254; 220 nm;) to give the title compound (117.6 mg, 63%) obtained as a white solid. MS (ESI, pos. ion) m/z: 329.2 (M+1). ¹H NMR (300 MHz, DMSO-d₆ ppm) δ 10.62 (d, J=1.7 Hz, 1H), 8.58 (t, J=5.9 Hz, 1H), 8.50 (s, 1H), 8.38 (d, J=1.5 Hz, 1H), 8.25 (s, 1H), 7.98 (s, 2H), 7.84-7.69 (m, 2H), 7.30 (d, J=7.7 Hz, 2H), 4.76 (d, J=6.0 Hz, 2H).

### Example 24

### Synthesis of 4-([thieno[3,2-d]pyrimidin-4-ylamino]methyl)phenylboronic acid

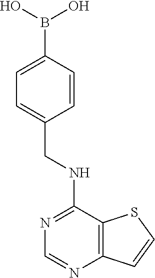 [SMILES: OB(O)c1ccc(CNc2ncnc3ccsc23)cc1]

The title compound was synthesized by the same method as described in example 10, step 2 except 4-chlorothieno[3,2-d]pyrimidine (100 mg, 0.59 mmol) and 4-(aminomethyl)phenylboronic acid hydrochloride (154 mg, 0.82 mmol) were used. The crude product was purified by prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 1% B to 9% B in 7 min, 9% B; Wave Length: 254/220 nm;) to give the title compound (104.9 mg, 63%) as a white solid. MS (ESI, pos. ion) m/z: 286.2 (M+1). ¹H NMR (300 MHz, DMSO-d₆, ppm) δ 8.44 (d, J=3.3 Hz, 2H), 8.12 (d, J=5.3 Hz, 1H), 7.98 (s, 2H), 7.83-7.69 (m, 2H), 7.40 (d, J=5.4 Hz, 1H), 7.30 (d, J=7.8 Hz, 2H), 4.75 (d, J=5.9 Hz, 2H).

### Example 25

### Synthesis of 4-([5H-pyrrolo[3,2-d]pyrimidin-4-ylamino]methyl)phenylboronic acid

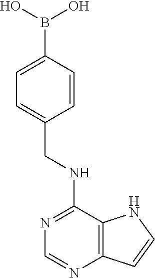 [SMILES: OB(O)c1ccc(CNc2ncnc3cc[nH]c23)cc1]

The title compound was synthesized by the same method as described in example 10, step 2 except 4-chloro-5H-pyrrolo[3,2-d]pyrimidine (100 mg, 0.65 mmol) and 4-(aminomethyl)phenyl-boronic acid hydrochloride (244 mg, 1.30 mmol) were used. The crude product was purified by prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 2% B to 10% B in 7 min, 10% B; Wave Length: 254; 220 nm;) to give the title compound (50.0 mg, 28%) as a white solid. MS (ESI, pos. ion) m/z: 269.2 (M+1). ¹H NMR (300 MHz, DMSO-d₆ and D₂O, ppm) δ 8.18 (s, 2H), 7.80-7.72 (m, 2H), 7.51 (d, J=3.0 Hz, 1H), 7.35 (d, J=7.8 Hz, 2H), 6.38 (d, J=3.0 Hz, 1H), 4.74 (s, 2H).

### Example 26

### Synthesis of 4-[([5-methylpyrrolo[3,2-d]pyrimidin-4-yl]amino)methyl]-phenyl-boronic acid formic acid

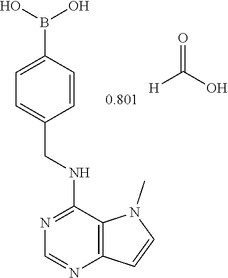 [SMILES: Cn1ccc2ncnc(NCc3ccc(B(O)O)cc3)c21 ; O=CO]

The title compound was synthesized by the same method as described in example 10, step 2 except 4-chloro-5-methylpyrrolo[3,2-d]pyrimidine (100 mg, 0.60 mmol) and 4-(aminomethyl)phenyl-boronic acid hydrochloride (157 mg, 0.84 mmol) were used. The crude product was purified by prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 2% B to 8% B in 7 min, 8% B; Wave Length: 254/220 nm;) to give The title compound (62.3 mg, 31%) was obtained as a white solid. MS (ESI, pos. ion) m/z: 283.3 (M+1). ¹H NMR (300 MHz, DMSO-d₆ ppm) δ 8.15 (s, 1H), 8.09 (s, 1H), 7.97 (s, 2H), 7.72 (d, J=7.9 Hz, 2H), 7.41 (d, J=3.0 Hz, 1H), 7.38-7.26 (m, 3H), 6.31 (d, J=3.0 Hz, 1H), 4.74 (d, J=5.8 Hz, 2H), 4.09 (s, 3H).

### Example 27

### Synthesis of 4-[([5-isopropyl-1H-pyrazolo[4,3-d]pyrimidin-7-yl]amino)methyl]phenyl boronic acid formic acid salt

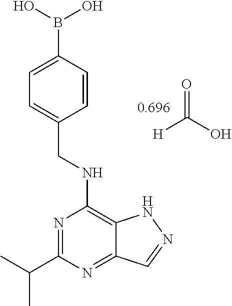 [SMILES: CC(C)c1nc(NCc2ccc(B(O)O)cc2)c2[nH]ncc2n1 ; O=CO]

### Step 1: methyl 2-benzyl-4-nitropyrazole-3-carboxylate

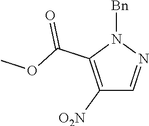 [SMILES: COC(=O)c1c([N+](=O)[O-])cnn1Cc1ccccc1]

Cesium carbonate (19.04 g, 58.44 mmol, 2.0 equiv) and benzyl bromide (7.50 g, 43.83 mmol, 1.5 equiv) were added to a solution of methyl 4-nitro-2H-pyrazole-3-carboxylate (5.00 g, 29.22 mmol, 1.0 equiv) in DMF (30 mL) at room temperature. After stirring overnight at room temperature, the reaction mixture was filtered. The filter cake was washed with ethyl acetate. The filtrate was washed with water and brine (300 ml), dried over anhydrous sodium sulfate, filtered and concentrated under reduced pressure. The residue was purified by column chromatography on silica gel (eluent: petroleum ether-ethyl acetate 100%, 70: 30) to afford the title compound (5.00 g, 66%) as a white solid.

### Step 2: methyl 4-amino-2-benzylpyrazole-3-carboxylate

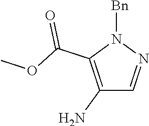 [SMILES: COC(=O)c1c(N)cnn1Cc1ccccc1]

To a solution of methyl 2-benzyl-4-nitropyrazole-3-carboxylate (450 mg, 1.72 mmol, 1.0 equiv) in ethanol (9 mL) and water (3 mL) was added Fe (962 mg, 17.23 mmol, 10.0 equiv) and NH₄Cl (921 mg, 17.23 mmol, 10.0 equiv) at room temperature under a nitrogen atmosphere. The resulting mixture was stirred at 80° C. for 2 h. After cooling to room temperature, the reaction mixture was filtered, and the filter cake was washed with EtOH and then concentrated under reduced pressure. The residue was purified by column chromatography on silica gel (eluent: petroleum ether-ethyl acetate 100%, 45: 55) to give the title compound (310 mg, 77%) as a purple solid.

### Step 3: methyl 2-benzyl-4-(2-methylpropanimidamido)pyrazole-3-carboxylate

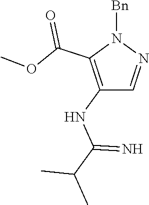 [SMILES: COC(=O)c1c(NC(=N)C(C)C)cnn1Cc1ccccc1]

To a solution of methyl 4-amino-2-benzylpyrazole-3-carboxylate (240 mg, 1.04 mmol, 1.0 equiv) in isobutyronitrile (5 mL) was added a solution of hydrogen chloride in 1,4-dioxane (2 mL, 4 M) at room temperature. The resulting mixture was stirred in a sealed tube at 100° C. for 4 h. The reaction mixture was cooled to room temperature and concentrated under reduced pressure. The residue was purified by column chromatography on silica gel 80 g (eluent: dichloromethane-methanol 100%, 85: 15) to give the title compound (268 mg, 85%) as a white solid.

### Step 4: 1-benzyl-5-isopropyl-6H-pyrazolo[4,3-d]pyrimidin-7-one

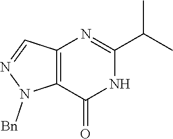 [SMILES: CC(C)c1nc2cnn(Cc3ccccc3)c2c(=O)[nH]1]

To a solution of methyl 2-benzyl-4-(2-methylpropanimidamido)pyrazole-3-carboxylate (248 mg, 0.82 mmol, 1.00 equiv) in ethanol (6 mL) was added NaOH (1 mL, 6 M in water) at room temperature. After stirring at 90° C. for 2 h, the reaction mixture was concentrated under reduced pressure. The residue was purified by column chromatography on silica gel 80 g (eluent: dichloromethane-methanol 100%, 94: 6) to the title compound (140 mg, 63%) as a white solid.

### Step 5: 5-isopropyl-1H,6H-pyrazolo[4,3-d]pyrimidin-7-one

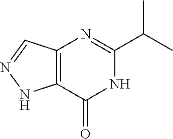

To a solution of 1-benzyl-5-isopropyl-6H-pyrazolo[4,3-d]pyrimidin-7-one (0.6 g, 2.23 mmol) in ethanol (10 mL) were added Pd/C (238 mg, 0.22 mmol) and 1N HCl aqueous solution (1 mL). After stirring overnight at 65° C. under hydrogen atmosphere, the reaction mixture was cooled to room temperature and filtered through a Celite. The filter cake was washed with ethanol and the filtrate was concentrated under reduced pressure to afford 5-isopropyl-1H,6H-pyrazolo[4,3-d]pyrimidin-7-one (205 mg, 51%) as a grey solid.

### Step 6: 7-chloro-5-isopropyl-1H-pyrazolo[4,3-d]pyrimidine

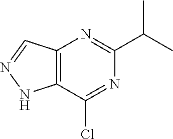

To a solution of 5-isopropyl-1H,6H-pyrazolo[4,3-d]pyrimidin-7-one (170 mg, 0.95 mmol, 1.0 equiv) in SOCl₂ (5 mL) was added DMF (0.2 mL) at room temperature. After stirring at 80° C. for 1.5 h, the reaction mixture was cooled to room temperature and concentrated under reduced pressure. The residue was purified by CombiFlash with the following conditions: ACN in water, 5% to 40% gradient in 15 min; detector, UV 254/220 nm., to give the title compound (90 mg, 45%) as a white solid.

### Step 7: 4-[([5-isopropyl-1H-pyrazolo[4,3-d]pyrimidin-7-yl]amino)methyl]phenylboronic acid formic acid salt

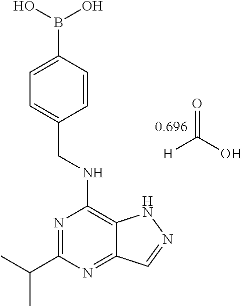 [SMILES: CC(C)c1nc(NCc2ccc(B(O)O)cc2)c2[nH]ncc2n1 ; O=CO]

To a solution of 7-chloro-5-isopropyl-1H-pyrazolo[4,3-d]pyrimidine (40 mg, 0.20 mmol, 1.0 equiv) in ethanol (5 mL) were added 4-(aminomethyl)phenylboronic acid hydrochloride (46 mg, 0.24 mmol, 1.2 equiv) and TEA (42 mg, 0.40 mmol, 2.0 equiv). After stirring overnight at 80° C., the reaction mixture was concentrated under reduced pressure. The crude product was purified by prep-HPLC with the following conditions (Column: Xselect CSH OBD Column 30*150 mm um; Mobile Phase A: Water (0.1% FA), Mobile Phase B: ACN; Flow rate: 60 mL/min; Gradient: 6% B to 19% B in 7 min, 19% B; Wave Length: 254/220 nm;) to afford the title compound (29.2 mg, 46%) as a white solid. MS (ESI, pos. ion) m/z: 312.3 (M+1). ¹H NMR (300 MHz, DMSO-d₆) δ 12.61 (brs, 1H), 8.14 (s, 0.696H), 8.01 (s, 2H), 7.76 (d, J=7.8 Hz, 2H), 7.39 (d, J=7.6 Hz, 2H), 4.75 (d, J=5.6 Hz, 2H), 2.85-3.00 (m, 1H), 1.23 (d, J=6.9 Hz, 6H).

### Example 28

### Synthesis of 4-[([5-phenyl-1H-pyrazolo[4,3-d]pyrimidin-7-yl]amino)methyl]phenylboronic acid

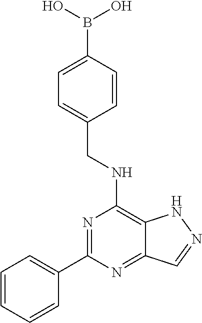 [SMILES: OB(O)c1ccc(CNc2nc(-c3ccccc3)nc3cn[nH]c23)cc1]

### Step 1: 4-nitro-2H-pyrazole-3-carboxamide

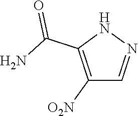 [SMILES: NC(=O)c1[nH]ncc1[N+](=O)[O-]]

A solution of methyl 4-nitro-2H-pyrazole-3-carboxylate (1.00 g, 5.84 mmol, 1.00 equiv) in NH₃—H₂O (15 mL) was stirred at 80° C. for 2 h in a sealed tube. After cooling to room temperature, the reaction mixture was concentrated under reduced pressure. The crude product was purified by column chromatography on silica gel 80 g (eluent: dichloromethane-methanol 100%, 95: 5) to afford the title compound (520 mg, 57%) as an off-white solid.

### Step 2: 4-amino-2H-pyrazole-3-carboxamide

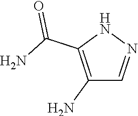

To a mixture of 4-nitro-2H-pyrazole-3-carboxamide (520 mg, 3.33 mmol) in methanol (10 mL) was added Pd/C (135 mg). After stirring at room temperature for 12 h under hydrogen atmosphere, the reaction mixture was filtered through a Celite. The filter cake was washed with methanol. The filtrate was concentrated under reduced pressure to give the title compound (350 mg, 83%) as an off-white solid.

### Step 3: 5-phenyl-1H,6H-pyrazolo[4,3-d]pyrimidin-7-one

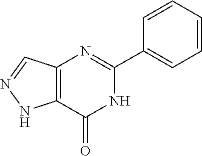 [SMILES: O=c1[nH]c(-c2ccccc2)nc2cn[nH]c12]

To a solution of 4-amino-2H-pyrazole-3-carboxamide (210 mg, 1.66 mmol, 1. equiv) in AcOH (10 mL) were added benzaldehyde (353 mg, 3.33 mmol, 2.0 equiv) and DDQ (755 mg, 3.33 mmol, 2.0 equiv). The resulting mixture was irradiated at 150° C. for 1 h in microwave reactor. After cooling to room temperature, the reaction mixture was diluted with ethyl acetate and washed with water and brine. The organic layer was dried over anhydrous sodium sulfate, filtered and concentrated under reduced pressure. The residue was purified by CombiFlash with the following conditions: methanol in water, 5% to 20% gradient in 20 min; detector, UV 254/220 nm.) to give the title compound (100 mg, 28%) as a brown solid.

### Step 4: 7-chloro-5-phenyl-1H-pyrazolo[4,3-d]pyrimidine

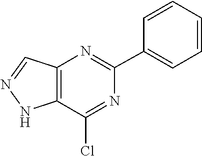 [SMILES: Clc1nc(-c2ccccc2)nc2cn[nH]c12]

This compound was synthesized by the same method as described in Example 27 Step 6, except 5-phenyl-1H,6H-pyrazolo[4,3-d]pyrimidin-7-one (100 mg, 0.47 mmol) was used. The title compound (80 mg, 74%) was obtained as a white solid.

### Step 5: 4-[([5-phenyl-1H-pyrazolo[4,3-d]pyrimidin-7-yl]amino)methyl]phenylboronic acid

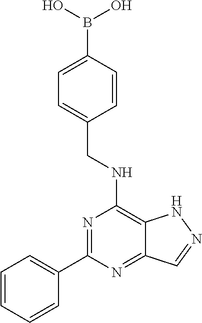 [SMILES: OB(O)c1ccc(CNc2nc(-c3ccccc3)nc3cn[nH]c23)cc1]

The title compound was synthesized by proceeding as described in Example 27, Step 7 except 7-chloro-5-phenyl-1H-pyrazolo[4,3-d]pyrimidine (80 mg, 0.35 mmol)) was used. The crude product was purified by prep-HPLC with the following conditions: (Column: Xselect CSH OBD Column 30*150 mm, Sum; Mobile Phase A: Water (0.1I % FA), Mobile Phase B: ACN; Flow rate: 60 mL/min; Gradient: 10% B to 28% B in 7 min, 28% B; Wave Length: 254; 220 nm). The collected fractions were combined and concentrated to give the title compound (27.2 mg, 22%) as a white solid. MS (ESI, pos. ion) m/z: 346.3 (M+1). ¹H NMR (400 MHz, DMSO-d₆, ppm) δ 12.77 (brs, 1H), 8.39-8.37 (m, 2H), 8.14 (s, 2H), 8.06-7.95 (m, 2H), 7.78-7.70 (m, 2H), 7.46-4.38 (m, 5H), 4.90 (s, 2H).

### Example 29

### Synthesis of 4-[([1-methyl-5-phenylpyrazolo[4,3-d]pyrimidin-7-yl]amino)-methyl]phenylboronic acid

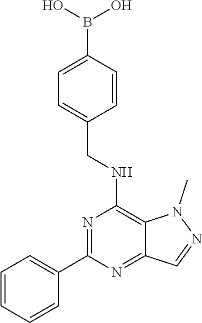 [SMILES: Cn1ncc2nc(-c3ccccc3)nc(NCc3ccc(B(O)O)cc3)c21]

### Step 1: 2-methyl-4-nitropyrazole-3-carboxamide

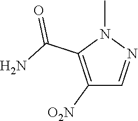

The title compound was synthesized by proceeding as described in Example 28, Step 1 except methyl 2-methyl-4-nitropyrazole-3-carboxylate (18.00 g, 97.22 mmol) was used. The title compound (12.50 g, 75%) was obtained as an off-white solid.

### Step 2: 4-amino-2-methylpyrazole-3-carboxamide

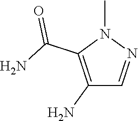

The title compound was synthesized by proceeding as described in Example 28, Step 2 except 2-methyl-4-nitropyrazole-3-carboxamide (12.50 g, 73.47 mmol) was used. The title compound (9.00 g, 87%) was obtained as an off-white solid.

### Step 3: 1-methyl-5-phenyl-6H-pyrazolo[4,3-d]pyrimidin-7-one

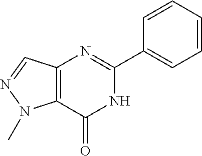 [SMILES: Cn1ncc2nc(-c3ccccc3)[nH]c(=O)c21]

The title compound was synthesized by proceeding as described in Example 28, Step 3 except 4-amino-2-methylpyrazole-3-carboxamide (300 mg, 2.14 mmol) was used and the reaction mixture was stirred at 120° C. for 1 h. The title compound (450 mg, 92%) was obtained as an off-white solid.

### Step 4: 7-chloro-1-methyl-5-phenylpyrazolo[4,3-d]pyrimidine

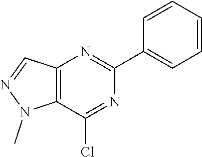 [SMILES: Cn1ncc2nc(-c3ccccc3)nc(Cl)c21]

The title compound was synthesized by proceeding as described in Example 27, Step 6 except 1-methyl-5-phenyl-6H-pyrazolo[4,3-d]pyrimidin-7-one (430 mg, 1.90 mmol) was used. The title compound (287 mg, 61%) was obtained as a brown yellow solid.

### Step 5: 4-[([1-methyl-5-phenylpyrazolo[4,3-d]pyrimidin-7-yl]amino)methyl]phenylboronic acid

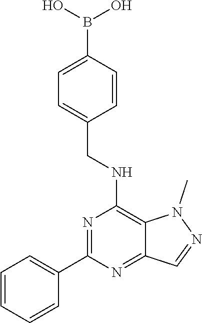 [SMILES: Cn1ncc2nc(-c3ccccc3)nc(NCc3ccc(B(O)O)cc3)c21]

The title compound was synthesized by proceeding as described in Example 27, Step 7 except 7-chloro-1-methyl-5-phenylpyrazolo[4,3-d]pyrimidine (110 mg, 0.45 mmol) was used. The crude product was purified by prep-HPLC with the following conditions (Column: Xselect CSH C18 OBD Column 30*150 mm, 5 m; Mobile Phase A: Water (0.1% FA), Mobile Phase B: ACN; Flow rate: 60 mL/min; Gradient: 15% B to 30% B in 7 min, 30% B; Wave Length: 254/220 nm). The collected fractions were combined and concentrated to afford the title compound (93.5 mg, 57%) as an off-white solid. MS (ESI, pos. ion) m/z: 360.0 (M+1). ¹H NMR (400 MHz, DMSO-d₆, ppm) δ 8.39-8.27 (m, 2H), 8.09-8.02 (m, 2H), 7.96 (s, 2H), 7.75-7.73 (m, 2H), 7.47-7.30 (m, 5H), 4.89-4.88 (m, 2H), 4.33 (s, 3H).

### Example 30

### Synthesis of 4-([[1-methyl-5-(pyridin-2-yl)pyrazolo[4,3-d]pyrimidin-7-yl]amino]-methyl)phenylboronic acid

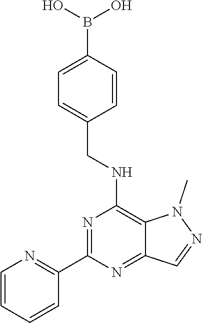 [SMILES: Cn1ncc2nc(-c3ccccn3)nc(NCc3ccc(B(O)O)cc3)c21]

### Step 1: 1-methyl-5-(pyridin-2-yl)-6H-pyrazolo[4,3-d]pyrimidin-7-one

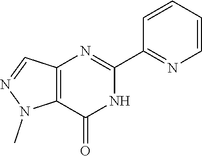 [SMILES: Cn1ncc2nc(-c3ccccn3)[nH]c(=O)c21]

The title compound was synthesized by proceeding as described in Example 28, Step 3 except 4-amino-2-methylpyrazole-3-carboxamide (300 mg, 2.14 mmol) and 2-formylpyridine (229 mg, 2.13 mmol) were used. The title compound (384 mg, 78%) was obtained as a brown solid.

### Step 2: 2-[7-chloro-1-methylpyrazolo[4,3-d]pyrimidin-5-yl]pyridine

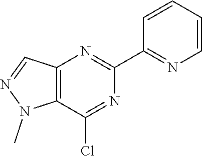 [SMILES: Cn1ncc2nc(-c3ccccn3)nc(Cl)c21]

To a mixture of 1-methyl-5-(pyridin-2-yl)-6H-pyrazolo[4,3-d]pyrimidin-7-one (62 mg, 0.27 mmol) in POCl₃ (3 mL) was added DMF (0.1 mL). After stirring at 90° C. for 5 h, the reaction mixture was cooled to room temperature and concentrated under reduced pressure. The residue was purified by reversed-phase flash chromatography with the following conditions: column, C18; mobile phase, ACN in water, 5% to 30% gradient in 20 min; detector, UV 254/220 nm to afford the title compound (32 mg, 47%) as an off-white solid.

### Step 3: 4-([[1-methyl-5-(pyridin-2-yl)pyrazolo[4,3-d]pyrimidin-7-yl]amino]methyl)-phenylboronic acid

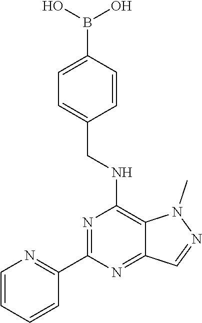 [SMILES: Cn1ncc2nc(-c3ccccn3)nc(NCc3ccc(B(O)O)cc3)c21]

The compound was synthesized by proceeding as described in Example 27, Step 7 except 2-[7-chloro-1-methylpyrazolo[4,3-d]pyrimidin-5-yl]pyridine (32 mg, 0.13 mmol) was used. The crude product was purified by prep-HPLC with the following conditions (Column: Sunfire prep C18 column, 30*150 mm, 5 m; Mobile Phase A: Water (0.1% FA), Mobile Phase B: ACN; Flow rate: 60 mL/min; Gradient: 3% B to 25% B in 9 min, 25% B; Wave Length: 254/220 nm). The collected fractions were combined and concentrated to afford the title compound (15.9 mg, 33%) as an off-white solid. (ESI, pos. ion) m/z: 361.1 (M+1). ¹H NMR (300 MHz, DMSO-d₆, ppm) 8.66 (m, 1H), 8.24-8.22 (m, 1H), 8.11-7.93 (m, 4H), 7.88-7.83 (m, 1H), 7.78-7.69 (m, 2H), 7.52-7.36 (m, 3H), 4.90 (d, J=5.5 Hz, 2H), 4.35 (s, 3H).

### Example 31

### Synthesis of 4-([[1-methyl-5-(pyridin-3-yl)pyrazolo[4,3-d]pyrimidin-7-yl]amino]methyl)-phenylboronic acid

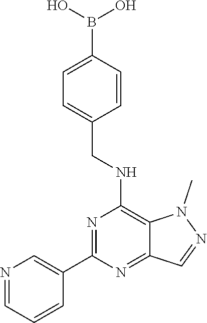 [SMILES: Cn1ncc2nc(-c3cccnc3)nc(NCc3ccc(B(O)O)cc3)c21]

### Step 1: 1-methyl-5-(pyridin-3-yl)-6H-pyrazolo[4,3-d]pyrimidin-7-one

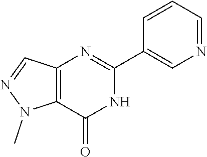 [SMILES: Cn1ncc2nc(-c3cccnc3)[nH]c(=O)c21]

The compound was synthesized by proceeding as described in Example 28, Step 3 except 4-amino-2-methylpyrazole-3-carboxamide (300 mg, 2.14 mmol) and 3-pyridinecarboxaldehyde (229 mg, 2.13 mmol) were used. The title compound (260 mg, 56%) was obtained as a light brown solid.

### Step 2: 3-[7-chloro-1-methylpyrazolo[4,3-d]pyrimidin-5-yl]pyridine

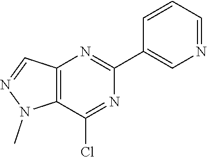 [SMILES: Cn1ncc2nc(-c3cccnc3)nc(Cl)c21]

The compound was synthesized by proceeding as described in Example 27, Step 6 except 1-methyl-5-(pyridin-3-yl)-6H-pyrazolo[4,3-d]pyrimidin-7-one (250 mg, 1.10 mmol) was used. The title compound (108 mg, 39%) was obtained as a brown solid.

### Step 3: 4-([[1-methyl-5-(pyridin-3-yl)pyrazolo[4,3-d]pyrimidin-7-yl]amino]methyl)phenyl-boronic acid

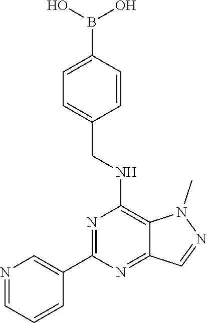 [SMILES: Cn1ncc2nc(-c3cccnc3)nc(NCc3ccc(B(O)O)cc3)c21]

The title compound was synthesized by proceeding as described in Example 27, Step 7 except 3-[7-chloro-1-methylpyrazolo[4,3-d]pyrimidin-5-yl]pyridine (60 mg, 0.24 mmol) was used. The crude product was purified by prep-HPLC with the following conditions (Column: XBridge Prep OBD C18 Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 18% B to 32% B in 7 min, 32% B; Wave Length: 254/220 nm). The collected fractions were combined and concentrated to afford the title compound (50.4 mg, 57%) as a white solid. MS (ESI, pos. ion) m/z: 361.0 (M+1). ¹H NMR (400 MHz, DMSO-d₆, ppm) δ 9.39-9.37 (m, 1H), 8.61-8.49 (m, 2H), 8.14 (t, J=5.8 Hz, 1H), 8.08 (s, 1H), 7.95 (s, 2H), 7.77-7.71 (m, 2H), 7.52-7.40 (m, 3H), 4.89 (d, J=5.8 Hz, 2H), 4.35 (s, 3H).

### Example 32

### Synthesis of 4-([[1-methyl-5-(pyridin-4-yl)pyrazolo[4,3-d]pyrimidin-7-yl]amino]methyl)phenylboronic acid

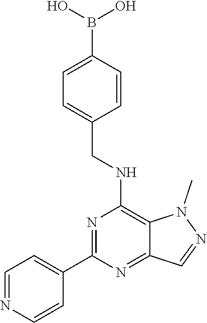 [SMILES: Cn1ncc2nc(-c3ccncc3)nc(NCc3ccc(B(O)O)cc3)c21]

### Step 1: 1-methyl-5-(pyridin-4-yl)-6H-pyrazolo[4,3-d]pyrimidin-7-one

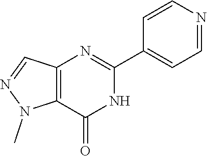 [SMILES: Cn1ncc2nc(-c3ccncc3)[nH]c(=O)c21]

The title compound was synthesized by proceeding as described in Example 28, Step 3 except 4-amino-2-methylpyrazole-3-carboxamide (300 mg, 2.14 mmol) and 4-formylpyridine (229 mg, 2.13 mmol) were used. The reaction mixture was stirred at 120° C. for 1 h. 1-Methyl-5-(pyridin-4-yl)-6H-pyrazolo[4,3-d]pyrimidin-7-one (470 mg, 58.%) was obtained as a yellow brown solid.

### Step 2: 4-[7-chloro-1-methylpyrazolo[4,3-d]pyrimidin-5-yl]pyridine

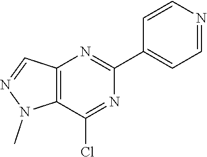 [SMILES: Cn1ncc2nc(-c3ccncc3)nc(Cl)c21]

The title compound was synthesized by proceeding as described in Example 27, Step 6 except 1-methyl-5-(pyridin-4-yl)-6H-pyrazolo[4,3-d]pyrimidin-7-one (450 mg, 1.98 mmol) was used. The title compound (212 mg, 43%) was obtained as a brown solid.

### Step 3: 4-([[1-methyl-5-(pyridin-4-yl)pyrazolo[4,3-d]pyrimidin-7-yl]amino]methyl)phenyl-boronic acid

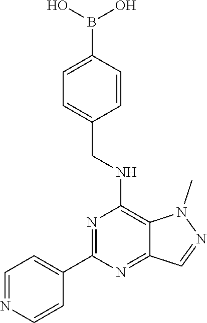 [SMILES: Cn1ncc2nc(-c3ccncc3)nc(NCc3ccc(B(O)O)cc3)c21]

The title compound was synthesized by proceeding as described in Example 27, Step 7 except 4-[7-chloro-1-methylpyrazolo[4,3-d]pyrimidin-5-yl]pyridine (110 mg, 0.44 mmol) was used. The residue was purified by prep-HPLC with the following conditions (Column: XBridge Prep OBD C18 Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 17% B to 32% B in 8 min, 32% B; Wave Length: 254/220 nm). The collected fractions were combined and concentrated to afford the title compound (69.7 mg, 43%) as an off-white solid. MS (ESI, pos. ion) m/z: 361.0 (M+1). ¹H NMR (400 MHz, DMSO-d₆, ppm) δ 8.67-8.63 (m, 2H), 8.21-8.11 (m, 4H), 7.98 (s, 2H), 7.74 (d, J=7.9 Hz, 2H), 7.47 (d, J=7.9 Hz, 2H), 4.90 (d, J=6.0 Hz, 2H), 4.35 (s, 3H).

### Example 33

### Synthesis of 4-([[1-methyl-5-(pyrazin-2-yl)pyrazolo[4,3-d]pyrimidin-7-yl]amino]methyl)-phenylboronic acid

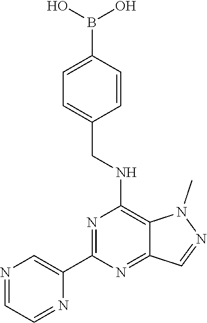 [SMILES: Cn1ncc2nc(-c3cnccn3)nc(NCc3ccc(B(O)O)cc3)c21]

### Step 1: 1-methyl-5-(pyrazin-2-yl)-6H-pyrazolo[4,3-d]pyrimidin-7-one

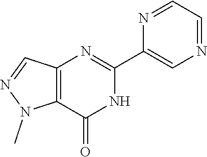 [SMILES: Cn1ncc2nc(-c3cnccn3)[nH]c(=O)c21]

The title compound was synthesized by proceeding as described in Example 28, Step 3 except 4-amino-2-methylpyrazole-3-carboxamide (300 mg, 2.14 mmol) and pyrazine-2-carbaldehyde (231 mg, 2.13 mmol) were used. The title compound (450 mg, 92%) was obtained as a yellow brown solid.

### Step 2: 2-[7-chloro-1-methylpyrazolo[4,3-d]pyrimidin-5-yl]pyrazine

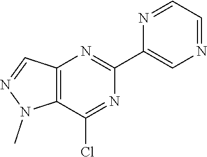 [SMILES: Cn1ncc2nc(-c3cnccn3)nc(Cl)c21]

A mixture of 1-methyl-5-(pyrazin-2-yl)-6H-pyrazolo[4,3-d]pyrimidin-7-one (200 mg, 0.87 mmol) in POCl₃ (10 mL) was stirred at 80 C for 2 h. The reaction mixture was cooled to room temperature and concentrated under reduced pressure. The residue was purified by reverse flash chromatography with the following conditions: column, C18; mobile phase, ACN in water, 5% to 100% gradient in 20 min; detector, UV 254/220 nm to afford the title compound (71 mg, 22%) as a brown yellow solid.

### Step 3: 4-([[1-methyl-5-(pyrazin-2-yl)pyrazolo[4,3-d]pyrimidin-7-yl]amino]methyl)-phenylboronic acid

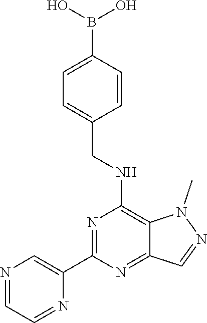 [SMILES: Cn1ncc2nc(-c3cnccn3)nc(NCc3ccc(B(O)O)cc3)c21]

The title compound was synthesized by proceeding as described in Example 27, Step 7 except 2-[7-chloro-1-methylpyrazolo[4,3-d]pyrimidin-5-yl]pyrazine (60 mg, 0.24 mmol) was used. The residue was purified by prep-HPLC with the following conditions (Column: Xselect CSH C18 OBD Column 30*150 mm, 5 m; Mobile Phase A: Water (0.1% FA), Mobile Phase B: ACN; Flow rate: 60 mL/min; Gradient: 5% B to 20% B in 7 min, 20% B; Wave Length: 254/220 nm). The collected fractions were combined and concentrated to afford the title compound (20.8 mg, 29%) as a white solid. MS (ESI, pos. ion) m/z: 362.0 (M+1). ¹H NMR (400 MHz, DMSO-d₆, ppm) δ 9.35 (d, J=1.5 Hz, 1H), 8.72 (s, 1H), 8.65 (s, 1H), 8.17-8.12 (m, 2H), 7.94 (s, 2H), 7.74 (d, J=7.9 Hz, 2H), 7.47 (d, J=7.7 Hz, 2H), 4.90 (d, J=5.7 Hz, 2H), 4.36 (s, 3H).

### Example 34

### Synthesis of 4-[([1-methyl-5-[2-phenylethenyl]pyrazolo[4,3-d]pyrimidin-7-yl]amino)-methyl]phenylboronic acid

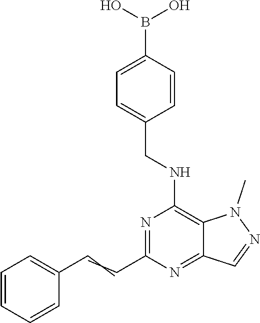 [SMILES: Cn1ncc2nc(C=Cc3ccccc3)nc(NCc3ccc(B(O)O)cc3)c21]

### Step 1: 2-methyl-4-[3-phenylprop-2-enamido]pyrazole-3-carboxamide

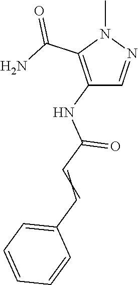 [SMILES: Cn1ncc(NC(=O)C=Cc2ccccc2)c1C(N)=O]

To a stirred solution of 4-amino-2-methylpyrazole-3-carboxamide (300 mg, 2.14 mmol, 1.0 equiv), cinnamic acid (317 mg, 2.14 mmol, 1.00 equiv) and HOBT (347 mg, 2.56 mmol, 1.2 equiv) in DMF (10 mL) were added EDCI (492 mg, 2.56 mmol, 1.2 equiv) and DIEA (829 mg, 6.42 mmol, 3 equiv) at room temperature. After stirring at room temperature for 3 h, the reaction mixture was concentrated under reduced pressure. The residue was purified by silica gel column chromatography, eluted with MeOH/DCM (1:20) to afford the title compound (450 mg, 77%) as an off-white solid.

### Step 2: 1-methyl-5-[2-phenylethenyl]-6H-pyrazolo[4,3-d]pyrimidin-7-one

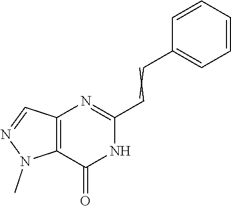 [SMILES: Cn1ncc2nc(C=Cc3ccccc3)[nH]c(=O)c21]

A mixture of 2-methyl-4-[3-phenylprop-2-enamido]pyrazole-3-carboxamide (200 mg, 0.74 mmol, 1.0 equiv) and NaOEt (201 mg, 2.96 mmol, 4.0 equiv) in ethanol (5 mL) was stirred overnight at 85° C. After cooling to room temperature, the reaction mixture was concentrated under reduced pressure, diluted ethyl acetate and washed with water and brine. The organic layer was dried over anhydrous sodium sulfate, filtered and concentrated under reduced pressure. The residue was purified by silica gel column chromatography, eluted with MeOH/DCM (1:20) to afford the title compound (160 mg, 74%) as a grey solid.

### Step 3: 7-chloro-1-methyl-5-[2-phenylethenyl]pyrazolo[4,3-d]pyrimidine

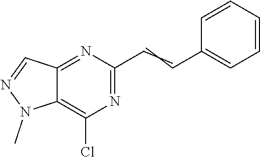 [SMILES: Cn1ncc2nc(C=Cc3ccccc3)nc(Cl)c21]

The title compound was synthesized by proceeding as described in Example 27, Step 6 except 1-methyl-5-[2-phenylethenyl]-6H-pyrazolo[4,3-d]pyrimidin-7-one (150 mg, 0.59 mmol) was used. The title compound (80 mg, 49%) was obtained as a light yellow solid.

### Step 4: 4-[([1-methyl-5-[2-phenylethenyl]pyrazolo[4,3-d]pyrimidin-7-yl]amino)methyl]-phenylboronic acid

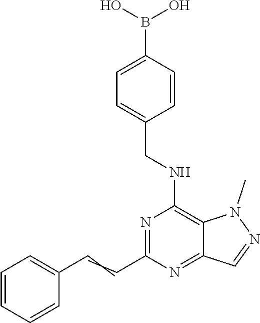 [SMILES: Cn1ncc2nc(C=Cc3ccccc3)nc(NCc3ccc(B(O)O)cc3)c21]

The compound was synthesized by proceeding as described in Example 27, Step 7 except 7-chloro-1-methyl-5-[2-phenylethenyl]pyrazolo[4,3-d]-pyrimidine (80 mg, 0.29 mmol) was used. The residue was purified by prep-HPLC with the following conditions (Column: Xselect CSH C18 OBD Column 30*150 mm, 5 m; Mobile Phase A: Water (0.1% FA), Mobile Phase B: ACN; Flow rate: 60 mL/min; Gradient: 14% B to 29% B in 7 min, 29% B; Wave Length: 254/220 nm). The collected fractions were combined and concentrated to afford the title compound as a mixture of E/Z isomers (20 mg, 17%) as a white solid, the E/Z-ratio was about 10:1. MS (ESI, pos. ion) m/z: 386.1 (M+1). ¹H NMR (300 MHz, DMSO-d₆, ppm) δ 7.99-7.94 (m, 4H), 7.77-7.74 (m, 2H), 7.70-7.57 (m, 3H), 7.52-7.28 (m, 5H), 7.23-6.91 (m, 1H), 4.84 (d, J=5.6 Hz, 2H), 4.32 (s, 3H).

### Example 35

### Synthesis of 4-[([1-methyl-5-[(E)-2-(pyridin-3-yl)ethenyl]pyrazolo[4,3-d]pyrimidin-7-yl]amino)methyl]phenylboronic acid

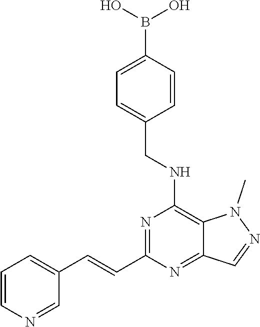 [SMILES: Cn1ncc2nc(/C=C/c3cccnc3)nc(NCc3ccc(B(O)O)cc3)c21]

### Step 1: 2-methyl-4-[(2E)-3-(pyridin-3-yl)prop-2-enamido]pyrazole-3-carboxamide

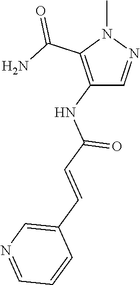 [SMILES: Cn1ncc(NC(=O)/C=C/c2cccnc2)c1C(N)=O]

The title compound was synthesized by proceeding as described in Example 34, Step 1 except (2E)-3-(pyridin-3-yl)prop-2-enoic acid (319 mg, 2.14 mmol) was used. The title compound (480 mg, 82%) was obtained as a grey solid.

### Step 2: 1-methyl-5-[(E)-2-(pyridin-3-yl)ethenyl]-6H-pyrazolo[4,3-d]pyrimidin-7-one

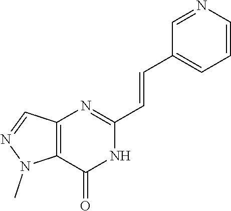 [SMILES: Cn1ncc2nc(/C=C/c3cccnc3)[nH]c(=O)c21]

The title compound was synthesized by proceeding as described in Example 34, Step 2 except 2-methyl-4-[(2E)-3-(pyridin-3-yl)prop-2-enamido]pyrazole-3-carboxamide (450 mg, 1.65 mmol) was used. The title compound (367 mg, 83%) was obtained as an off-white solid.

### Step 3: 3-[(E)-2-[7-chloro-1-methylpyrazolo[4,3-d]pyrimidin-5-yl]ethenyl]pyridine

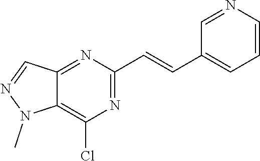 [SMILES: Cn1ncc2nc(/C=C/c3cccnc3)nc(Cl)c21]

The title compound was synthesized by proceeding as described in Example 27, Step 6 except 1-methyl-5-[(E)-2-(pyridin-3-yl)ethenyl]-6H-pyrazolo[4,3-d]pyrimidin-7-one (335 mg, 1.32 mmol) was used. The title compound (176 mg, 48%) was obtained as a yellow solid.

### Step 4: 4-[([1-methyl-5-[(E)-2-(pyridin-3-yl)ethenyl]pyrazolo[4,3-d]pyrimidin-7-yl]amino)-methyl]phenylboronic acid

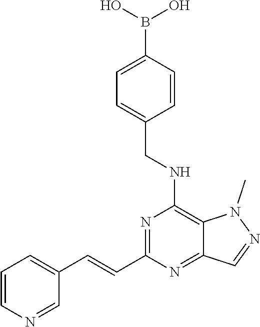 [SMILES: Cn1ncc2nc(/C=C/c3cccnc3)nc(NCc3ccc(B(O)O)cc3)c21]

The title compound was synthesized by proceeding as described in example 27 step 7 except 3-[(E)-2-[7-chloro-1-methylpyrazolo[4,3-d]pyrimidin-5-yl]-ethenyl]pyridine (77 mg, 0.28 mmo) was used. The residue was purified by prep-HPLC with the following conditions (Column: Sunfire prep C18 column, 30*150 mm, 5 m; Mobile Phase A: Water (0.1% FA), Mobile Phase B: MeOH—Preparative; Flow rate: 60 mL/min; Gradient: 15% B to 37% B in 10 min, 37% B; Wave Length: 254/220 nm). The collected fractions were combined and concentrated to afford the title compound (30.0 mg, 26%) as a white solid. MS (ESI, pos. ion) m/z: 387.2 (M+1). ¹H NMR (300 MHz, DMSO-d₆, ppm) δ 8.79 (d, J=2.2 Hz, 1H), 8.52 (d, J=4.8 Hz, 1H), 8.16-8.05 (m, 2H), 8.01-7.94 (m, 3H), 7.80-7.63 (m, 3H), 7.50-7.39 (m, 3H), 7.19-7.14 (m, 1H), 4.86 (d, J=5.5 Hz, 2H), 4.33 (s, 3H).

### Example 36

### Synthesis of 4-(2-[7-formamidothieno[3,2-d]pyrimidin-4-yl]ethynyl)phenylboronic acid

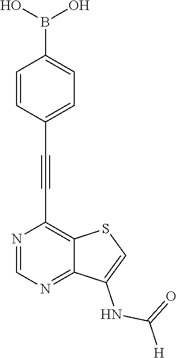 [SMILES: O=CNc1csc2c(C#Cc3ccc(B(O)O)cc3)ncnc12]

### Step 1: 4-bromo-7-nitrothieno[3,2-d]pyrimidine

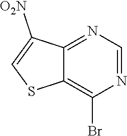 [SMILES: O=[N+]([O-])c1csc2c(Br)ncnc12]

To a solution of 7-nitrothieno[3,2-d]pyrimidin-4-ol (500 mg, 2.53 mmol, 1.0 equiv) in acetonitrile (50 mL) was added POBr₃ (7.27 g, 25.36 mmol, 10.0 equiv) at room temperature. The resulting mixture was stirred overnight at 110° C. After cooling the mixture to room temperature, the reaction mixture was concentrated under reduced pressure, then saturated sodium bicarbonate solution (50 mL) was added at 0° C. The resulting mixture was extracted with DCM. The combined organic layers were dried over anhydrous sodium sulfate. After filtration, the filtrate was concentrated under reduced pressure to afford the title compound (410 mg, 62%) as a light yellow solid.

### Step 2: 4-bromothieno[3,2-d]pyrimidin-7-amine

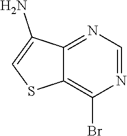 [SMILES: Nc1csc2c(Br)ncnc12]

To a stirred mixture of 4-bromo-7-nitrothieno[3,2-d]pyrimidine (380 mg, 1.46 mmol, 1.0 equiv) in ethanol (10 mL) and water (3 mL) were added Fe (815 mg, 14.61 mmol, 10.0 equiv) and NH₄Cl (781 mg, 14.61 mmol, 10.0 equiv) at room temperature. After stirring at 80° C. for 2 h, the reaction mixture was cooled to room temperature, filtered and the filter cake was washed with ethanol. The filtrate was concentrated under reduced pressure. The residue was purified by silica gel column chromatography, eluted with PE/EA (1:1) to afford the title compound (260 mg, 77%) as a brown solid.

### Step 3: N-[4-bromothieno[3,2-d]pyrimidin-7-yl]formamide

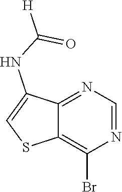 [SMILES: O=CNc1csc2c(Br)ncnc12]

A mixture of 4-bromothieno[3,2-d]pyrimidin-7-amine (240 mg, 1.04 mmol, 1.0 equiv) in formic acid (10 mL) was stirred at room temperature for 2 h. The reaction mixture was poured to a saturated sodium bicarbonate solution (50 mL) at 0° C. The resulting mixture was extracted with DCM. The combined organic layers were washed with saturated sodium chloride solution, dried over anhydrous sodium sulfate. After filtration, the filtrate was concentrated under reduced pressure to afford the title compound (220 mg, 89%) as a brown solid.

### Step 4: 4-(2-[7-formamidothieno[3,2-d]pyrimidin-4-yl]ethynyl)phenylboronic acid

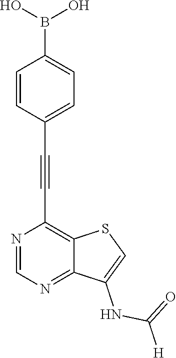 [SMILES: O=CNc1csc2c(C#Cc3ccc(B(O)O)cc3)ncnc12]

To a mixture of N-[4-bromothieno[3,2-d]pyrimidin-7-yl]formamide (90 mg, 0.34 mmol, 1.0 equiv) and 2-(4-ethynylphenyl)-4,4,5,5-tetramethyl-1,3,2-dioxaborolane (119 mg, 0.52 mmol, 1.5 equiv) in TEA (5 mL) were added CuI (7 mg, 0.03 mmol, 0.1 equiv) and dichloropalladium bis(triphenylphosphane) (24 mg, 0.03 mmol, 0.1 equiv) at room temperature. After stirring overnight at room temperature under nitrogen atmosphere, the reaction mixture was extracted with DCM. The combined organic layers were washed with saturated sodium chloride solution and dried over anhydrous sodium sulfate. After filtration, the filtrate was concentrated under reduced pressure. The crude product was purified by prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 25% B to 45% B in 10 min, 45% B; Wave Length: 254/220 nm;) to afford the title compound (4.7 mg, 4%) as a white solid. MS (ESI, pos. ion) m/z: 324.1 (M+1). ¹H NMR (400 MHz, DMSO-d₆, ppm) δ 11.02 (d, J=1.5 Hz, 1H), 9.29 (s, 1H), 8.68 (s, 1H), 8.45 (d, J=1.5 Hz, 1H), 8.34 (s, 2H), 7.93 (d, J=8.4 Hz, 2H), 7.72 (d, J=8.0 Hz, 2H).

### Example 37

### Synthesis of 4-([1H-pyrazolo[4,3-b]pyridin-7-ylamino]methyl)phenylboronic acid

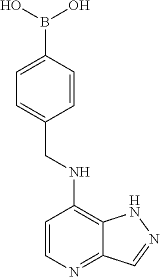 [SMILES: OB(O)c1ccc(CNc2ccnc3cn[nH]c23)cc1]

To a stirred mixture of 7-chloro-1H-pyrazolo[4,3-b]pyridine (100 mg, 0.65 mmol, 1.0 equiv) in NMP (6 mL) were added 1-[4-(4,4,5,5-tetramethyl-1,3,2-dioxaborolan-2-yl)phenyl]methanamine hydrochloride (364 mg, 1.35 mmol, 2.0 equiv) and CsF (148 mg, 0.97 mmol, 1.5 equiv) at room temperature. The resulting mixture was stirred overnight at 110° C. After cooling to room temperature, the mixture was purified by reverse flash chromatography with the following conditions: column, C18 silica gel; mobile phase, ACN in water, 50% to 100% gradient in 5 min; detector, UV 254 nm and 220 nm to afford 26 mg crude material as a brown solid, which was further purified by prep-HPLC with the following conditions (Column: XBridge Prep OBD C18 Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 9% B to 24% B in 8 min, 24% B; Wave Length: 254 & 220 nm;) to afford the title compound (2.9 mg, 1.6%) as a white solid. MS (ESI, pos. ion) m/z: 269.0 (M+1). ¹H NMR (400 MHz, DMSO-d₆, ppm) δ 12.63 (s, 1H), 8.20-8.05 (m, 4H), 7.78 (d, J=7.7 Hz, 2H), 7.38 (d, J=7.7 Hz, 2H), 7.00 (s, 1H), 6.31 (s, 1H), 4.52 (d, J=5.6 Hz, 2H).

### Example 38

### Synthesis of 4-([[7-(methoxycarbonyl)thieno[3,2-d]pyrimidin-4-yl]amino]methyl)-phenylboronic acid

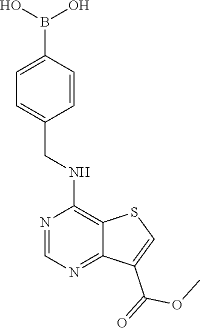 [SMILES: COC(=O)c1csc2c(NCc3ccc(B(O)O)cc3)ncnc12]

The title compound was synthesized by proceeding as described in Example 27, Step 7 except methyl 4-chlorothieno[3,2-d]pyrimidine-7-carboxylate (300 mg, 1.31 mmol) was used. The residue was purified by silica gel column chromatography, eluted with EA/PE (3:1) to afford the title compound (330 mg, 73%) as a light-yellow solid. MS (ESI, pos. ion) m/z: 344.0 (M+1). ¹H NMR (400 MHz, DMSO-d₆, ppm) δ 8.89 (s, 1H), 8.64 (t, J=5.9 Hz, 1H), 8.52 (s, 1H), 7.99 (s, 2H), 7.73 (d, J=8.0 Hz, 2H), 7.30 (J=8.4 Hz, 2H), 4.75 (d, J=5.9 Hz, 2H), 3.85 (s, 3H).

### Example 39

### Synthesis of 4-[([7-carbamoylthieno[3,2-d]pyrimidin-4-yl]amino)methyl]phenylboronic acid

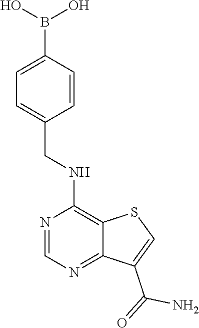 [SMILES: NC(=O)c1csc2c(NCc3ccc(B(O)O)cc3)ncnc12]

The title compound was synthesized by proceeding analogously as described in Example 28, Step 1 except 4-([[7-(methoxycarbonyl)thieno[3,2-d]pyrimidin-4-yl]amino]methyl)-phenylboronic acid (100 mg, 0.29 mmol) was used. The residue was purified by prep-HPLC with the following conditions (Column: Xselect CSH C18 OBD Column 30*150 mm, 5 m; Mobile Phase A: Water (0.1% FA), Mobile Phase B: ACN; Flow rate: 60 mL/min; Gradient: 8% B to 23% B in 7 min, 23% B; Wave Length: 254/220 nm) to afford the title compound (69.3 mg, 72%) as a white solid. MS (ESI, pos. ion) m/z: 329.0 (M+1). ¹H NMR (400 MHz, DMSO-d₆, ppm) δ 8.99 (d, J=3.6 Hz, 1H), 8.80 (t, J=5.9 Hz, 1H), 8.76 (s, 1H), 8.55 (s, 1H), 7.99 (s, 2H), 7.80 (t, J=4.4 Hz, 1H), 7.77-7.71 (m, 2H), 7.34-7.28 (m, 2H), 4.78 (d, J=5.9 Hz, 2H).

### Example 40

### Synthesis of 4-[([7-cyanothieno[3,2-d]pyrimidin-4-yl]amino)methyl]phenylboronic acid

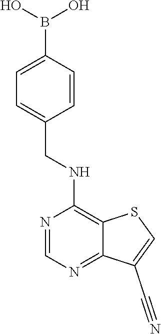 [SMILES: N#Cc1csc2c(NCc3ccc(B(O)O)cc3)ncnc12]

A solution of 4-[([7-carbamoylthieno[3,2-d]pyrimidin-4-yl]amino)methyl]phenylboronic acid (80 mg, 0.24 mmol) in POCl₃ (4 mL) was stirred at 80° C. for 2 h. After cooling to room temperature, the reaction mixture was concentrated under reduced pressure. The crude product was purified by prep-HPLC with the following conditions (Column: Xselect CSH C18 OBD Column 30*150 mm, 5 m; Mobile Phase A: Water (0.1% FA), Mobile Phase B: ACN; Flow rate: 60 mL/min; Gradient: 16% B to 27% B in 7 min, 27% B; Wave Length: 254/220 nm) to afford the title compound (9.9 mg, 12%) as a yellow solid. MS (ESI, pos. ion) m/z: 311.0 (M+1). ¹H NMR (300 MHz, DMSO-d₆, ppm) δ 9.12 (s, 1H), 8.93 (s, 1H), 8.54 (s, 1H), 7.74 (d, J=7.7 Hz, 2H), 7.31 (d, J=7.7 Hz, 2H), 4.76 (d, J=5.8 Hz, 2H)

### BIOLOGICAL EXAMPLES

### Example 1

### Measurement of pNP-TMP Hydrolysis by ENPP1

p-Nitrophenyl thymidine 5′-monophosphate (pNP-TMP) is a synthesized substrate for ENPP1. The ENPP1 enzyme activity assay with pNP-TMP substrate was conducted as follows:

First, in a 60 μl reaction, 7.5 ng purified ENPP1 was mixed with compounds of Formula (I) (test compound) ranging from 13.7 μM to 10 μM. Incubation of ENPP1 with compounds was set at 25° C. for 10 min. Reactions with DMSO only (with ENPP1 but no compound) gave the fastest reaction (MAX Activity). For each compound dilution, wells with assay buffer (50 mM Tris-HCl, pH8.8, 250 mM NaCl, 0.1 mg/ml BSA, 1% DMSO) only but no ENPP1 were included as controls for subtraction of test compound derived absorbance at 405 nm.

Second, after the 10 minutes ENPP1 and test compound incubation the assay was initiated by transferring 50 μl of the above mentioned ENPP1/test compound reaction into 50 μl of 1 mM pNP-TMP in assay buffer results in a 100 μl total reaction in clear bottom 96 well plates. Absorbance at 405 nm was recorded immediately in kinetic mode by PerkinElmer 2300 Enspire multimode plate reader.

For each inhibitor, the specific ENPP1 activity was calculated using the following equation: ENPP1 activity (pmol/min/g)=Adjusted Vmax (OD405 nm/min) X conversion factor (pmol/OD405 nm)/amount of enzyme (g)

Adjusted Vmax=V₀×(Km+(S))/(S). In this assay, Km=232 μM, (S)=500 μM. Adjusted Vmax=1.464×V₀.

V₀=(OD405 nm with ENPP1−OD405 nm ENPP1 blank)/minutes. OD405 nm was plotted, with blank subtracted, against time (minutes), the initial linear rate is V₀. blank subtracted, against time (minutes), the initial linear rate is V₀.

The conversion factor (pmol/OD405 nm), was determined by plotting the amount of standard, 4-Nitrophenol (Sigma-Aldrich, Catalog #241326), against absorbance at 405 nm. The slope is the conversion factor. The percent ENPP1 activity for each sample was calculated using the following equation:

% enzyme activity=sample enzyme activity/MAX Activity×100%.

To determine the IC₅₀ for each compound, compound concentration values and percent enzyme activity values were inserted into GraphPad Prism (GraphPad Prism version 7.0 for Windows, GraphPad Software, La Jolla California USA, www.graphpad.com), and Prism's Transform analysis was used to convert the x-axis values (compound concentration) to logarithms. A sigmoidal variable slope nonlinear regression analysis was performed using the following equation: Y=Bottom+(Top−Bottom)/(1+10\hat{ }((LogIC₅₀−X)*HillSlope)).

Kᵢ values for each compound were calculated from the observed IC₅₀ from GraphPad analysis using the Cheng-Prusoff equation: Kᵢ=IC₅₀/(1+(S)/K_{M}). (S) here is 500 μM and K_{M} is determined to be 232 μM.

Ki for a representative compound of Formula (I) in Compound Table 1 above is provided in Table 2 below:

TABLE 2
Ki (pNP-TMP)

| Group A | Group B | Group C | Group D |
|---|---|---|---|
| (<0.10 μM) | (<1 μM) | (<10 μM) | (>10 μM) |
| Cpd. 8, 25, 1, 6, | Cpd. 2, 3, 5, 7, | Cpd. 4, 13, | Cpd. 22, 23, 24 |
| 9, 10, 12, 15, 17, | 11, 14, 16, 27, 28, | 20, 21, | |
| 18, 19, 26, 30, | 29, 31, 34, 36, | | |
| 32, 33, 35, 37, | | | |
| 38, 39, 40 | | | |

### Example 2

### Measurement of 2′3′-cGAMP Hydrolysis by ENPP1

ENPP1 catalyzes the hydrolysis of 2′3′-cGAMP into 5′-AMP and 5′-GMP, and hence the ENPP1 enzyme activity with 2′3′-cGAMP as substrate is monitored by measurement of the product 5′-AMP. The AMP-Glo assay kit from Promega (catalog number V5012) is used for measurement of 5′-AMP production.

First, an ENPP1 and test compound incubation is set up in assay buffer (50 mM Tris-HCl, pH8.8, 250 mM NaCl, 0.1 mg/ml BSA, 1% DMSO) with following conditions: ENPP1 concentration: 1.25 nM; test compound concentration ranging from 68 μM to 20 μM. This incubation is carried out at 25° C. for 10 min.

Second, after the 10 minute ENPP1 and test compound incubation, prepare on a separate plate, 15 μl of the substrate 2′3′-cGAMP at 200 μM in assay buffer. Then, 15 μl of the ENPP1/Compound incubation is transferred to the 200 μM 2′3′-cGAMP solution to initiate the reaction. The 30 μl mixture is incubated for 30 min at 25° C. In all these assays a DMSO control without compound is included which gave the maximum 5′-AMP production (MAX RLU). After 30 min the reaction is stopped by heating at 90° C. for 3 min.

Third, the Promega AMP-Glo kit is used to detect 5′-AMP production as a measurement of ENPP1 enzyme activity. To do this 10 μl of the above mentioned 30 μl total reaction per sample is transferred into 384 well white solid assay plate for measurement of 5′-AMP production. For each well, 10 μl of AMP-Glo Reagent I is added, mixed well, and incubated for 1 hour at 25° C. At this time AMP detection solution is prepared and 20 μl is added per well, and the resulting solution is incubated for 1 hr at 25° C. Duplicates are run for each inhibitor concentration. Luminescence signal (relative luminescence units, RLU) is recorded using a PerkinElmer 2300 Enspire multimode plate reader.

The % inhibition is calculated using the following equation: % inhibition=(MAX RLU−sample RLU)/MAX RLU X 100%.

IC₅₀ values of compounds are determined by loading compound concentration data and percent inhibition values into GraphPad Prism (GraphPad Prism version 7.0 for Windows, GraphPad Software, La Jolla California USA, www.graphpad.com) and conducted a Sigmoidal variable slope nonlinear regression fitting.

Kᵢ values for each compound are calculated from the observed IC₅₀ from GraphPad analysis using the Cheng-Prusoff equation: Kᵢ=IC₅₀/(1+(S)/K_{M}). (S) here is 100 μM and K_{M} is 32 M.

### Formulation Examples

The following are representative pharmaceutical formulations containing a compound of the present disclosure.

### Tablet Formulation

The following ingredients are mixed intimately and pressed into single scored tablets.

| | Quantity per tablet |
|---|---|
| Ingredient | mg |
| compound of this disclosure | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per capsule |
|---|---|
| Ingredient | mg |
| compound of this disclosure | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL.

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 ul of spray for each application.

What is claimed:

1. A compound of Formula (I): 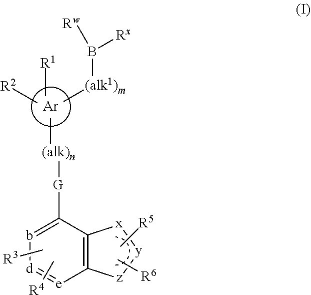
wherein: [symbol]
dashed line is a bond between x and y or y and z;
b, d, and e are CH; or one or two of b, d, and e are N and remaining of b, d, and e are CH;
one of x and z is NH, 0, or S and the other of x and z is CH or N; and y is CH or N;
provided that, at least one of y and the x or z that is CH or N, is CH;
G is a bond, NR (where R is hydrogen or alkyl), O, S, or SO₂;
alk is alkylene optionally substituted with one, two, or three halo, or alkynylene, provided that when alk is alkynylene G is a bond and n is 1;
alk¹ is alkylene optionally substituted with one, two, or three halo;
m and n are independently 0 or 1; provided that at least one of m and n is 1;
Ar is aryl or heteroaryl;
R^{w} and R^{x} are independently selected from hydroxy, alkoxy, —Oaryl (where aryl is optionally substituted with one to three substituents independently selected from alkyl, alkenyl, alkoxy, halo, haloalkyl, amino, alkylamino, dialkylamino, cyano, and nitro), —O—(CH₂)OCOR^{a}(where R^{a} is alkyl), —O-(alk²)OR (where alk² is alkylene and R^{b} is alkyl), —S—(CH₂)₂SCOR^{c} (where R^{c} is alkyl), or —NR^{e}—(CHR^{d})OCOR^{f} (where R^{d} is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl either alone or as part of phenylalkyl is optionally substituted with one to three substituents independently selected from alkyl, alkoxy, halo, hydroxy, cyano and nitro), R is alkyl or benzyl and R^{e} is hydrogen or R^{e} together with R^{d} forms —(CH₂)₃—); or
R^{w} and R^{x} together with the boron atom to which they are attached can form a ring selected from: 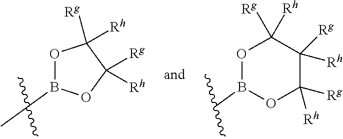
wherein each R⁹ and R^{h} is independently hydrogen or methyl;
R¹ and R² are independently absent, alkyl, alkoxy, halo, haloalkyl, haloalkoxy, or cyano;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl, either alone or part of heterocyclyloxy, and heterocyclylamino is optionally substituted with Rⁱ, R^{j}, or R^{k} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyl, heteroaryl, phenylalkenyl, heteroarylalkenyl, phenyloxy, or heteroaryloxy (where phenyl, by itself or as part of phenylalkenyl and phenyloxy and heteroaryl, by itself or as part of heteroarylalkenyl and heteroaryloxy, are optionally substituted with one, two, or three substituents where two of the optional substituents are independently selected from alkyl, hydroxy, alkoxy, halo, haloalkyl, haloalkoxy, and cyano); provided that (i) when two of b, d, and e are N, then at least one of R³ and R⁴ is absent;
R⁵ is absent, alkyl, hydroxy, alkoxy, alkoxycarbonyl, halo, acylamino, hydroxyalkylamino, alkoxyalkylamino, cyano, aminocarbonyl, alkylaminocarbonyl, dialkylaminocarbonyl, alkoxyalkyloxycarbonyl, aminoalkyloxycarbonyl, or hydroxyalkylaminocarbonyl; and
R⁶ is absent or alkyl; provided that one of R⁵ and R⁶ is absent when two of x, y, and z are other than CH; or
a pharmaceutically acceptable salt thereof,
provided that: (i) when b, e, x, and z are N, d and y are CH, G is S, n is 1, m is 0, R⁵ and R⁶ are absent, and one of R³ and R⁴ is absent, then the other of R³ and R⁴ is present; and (ii) the compound of Formula (I) is not (3-(((2-amino-7H-purin-6-yl)oxy)methyl)phenyl)boronic acid or 7-((4-(4,4,5,5-tetramethyl-1,3,2-dioxaborolan-2-yl)-1H-pyrazol-1-yl)methyl)-1H-indazole.

2. A compound of Formula (I): 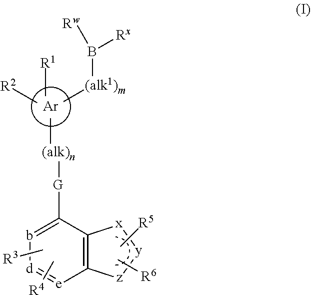
wherein: [symbol]
dashed line is a bond between x and y or y and z;
b, d, and e are CH; or one or two of b, d, and e are N and remaining of b, d, and e are CH;
one of x and z is NH, O, or S and the other of x and z is CH or N; and y is CH or N; provided that, at least one of y and the x or z that is CH or N, is CH;
G is a bond, NR (where R is hydrogen or alkyl), O, S, or SO₂;
alk is alkylene optionally substituted with one, two, or three halo;
alk¹ is alkylene optionally substituted with one, two, or three halo;
m and n are independently 0 or 1; provided that at least one of m and n is 1;
Ar is aryl or heteroaryl;
R^{w} and R^{x} are independently selected from hydroxy, alkoxy, —Oaryl (where aryl is optionally substituted with one to three substituents independently selected from alkyl, alkenyl, alkoxy, halo, haloalkyl, amino, alkylamino, dialkylamino, cyano, and nitro), —O—(CH₂)OCOR^{a}(where R^{a} is alkyl), —O-(alk²)OR (where alk² is alkylene and R^{b} is alkyl), —S—(CH₂)₂SCOR^{c} (where R^{c} is alkyl), or —NR^{e}—(CHR^{d})OCOR^{f} (where R^{d} is hydrogen, alkyl, hydroxymethyl, thiomethyl, methylthiomethyl, amidinopropyl, indol-3-ylmethyl, indol-4-ylmethyl, carboxymethyl, carboxyethyl, aminocarbonylmethyl, aminocarbonylethyl, phenyl or phenylalkyl (wherein phenyl either alone or as part of phenylalkyl is optionally substituted with one to three substituents independently selected from alkyl, alkoxy, halo, hydroxy, cyano and nitro), R is alkyl or benzyl and R^{e} is hydrogen or R^{e} together with R^{d} forms —(CH₂)₃—); or
R^{w} and R^{x} together with the boron atom to which they are attached can form a ring selected from: 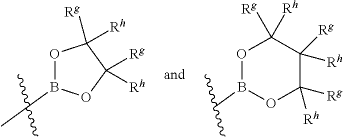
wherein each R^{g} and R^{h} is independently hydrogen or methyl;
R¹ and R² are independently absent, alkyl, alkoxy, halo, haloalkyl, haloalkoxy, or cyano;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl, either alone or part of heterocyclyloxy, and heterocyclylamino is optionally substituted with Rⁱ, R^{j}, or R^{k} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents where two of the optional substituents are independently selected from alkyl, hydroxy, alkoxy, halo, haloalkyl, haloalkoxy, and cyano); provided that (i) when two of b, d, and e are N, then at least one of R³ and R⁴ is absent and (ii) when G is S and one of R³ and R⁴ is absent, then the other of R³ and R⁴ is present and is other than amino;
R⁵ is absent, alkyl, hydroxy, halo, acylamino, hydroxyalkylamino, alkoxyalkylamino, cyano, aminocarbonyl, alkylaminocarbonyl, or dialkylaminocarbonyl; and
R⁶ is absent or alkyl; provided that one of R⁵ and R⁶ is absent when two of x, y, and z are other than CH; or
a pharmaceutically acceptable salt thereof,
provided that: (i) when b, e, x, and z are N, d and y are CH, G is S, n is 1, m is 0, R⁵ and R⁶ are absent, and one of R³ and R⁴ is absent, then the other of R³ and R⁴ is present; and (ii) the compound of Formula (I) is not (3-(((2-amino-7H-purin-6-yl)oxy)methyl)phenyl)boronic acid or 7-((4-(4,4,5,5-tetramethyl-1,3,2-dioxaborolan-2-yl)-1H-pyrazol-1-yl)methyl)-1H-indazole.

3. The compound of claim 1, or a pharmaceutically acceptable salt thereof, wherein. x is NH, O, or S;
G is NR (where R is hydrogen or alkyl), O, or S;
alk is alkylene optionally substituted with one, two, or three halo;
R³ and R⁴ are independently absent, alkyl, alkoxy, hydroxy, halo, haloalkyl, haloalkoxy, alkylsulfonyl, hydroxyalkyl, alkoxyalkyl, hydroxyalkoxy, alkoxyalkoxy, hydroxyalkylamino, alkoxyalkylamino, amino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with Rⁱ, R^{j}, or R^{k} independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents where two of the optional substituents are independently selected from alkyl, hydroxy, alkoxy, halo, haloalkyl, haloalkoxy, and cyano); provided that (i) when two of b, d, and e are N, then one of R³ and R⁴ is absent and (ii) when G is S and one of R³ and R⁴ is absent, then the other of R³ and R⁴ is present and other than amino;
R⁵ is absent, alkyl, hydroxy, halo, acylamino, hydroxyalkylamino, alkoxyalkylamino, aminocarbonyl, alkylaminocarbonyl, or dialkylaminocarbonyl.

4. The compound of claim 3, or pharmaceutically acceptable salt thereof, wherein:
x is NH, O, or S;
y and z are independently CH or N; provided that at least one of y and z is CH; and
G is NR (where R is hydrogen or alkyl) or O.

5. The compound of any one of claim 3 or 4, or a pharmaceutically acceptable salt thereof, having a structure of formula (Ia) or (Ib):

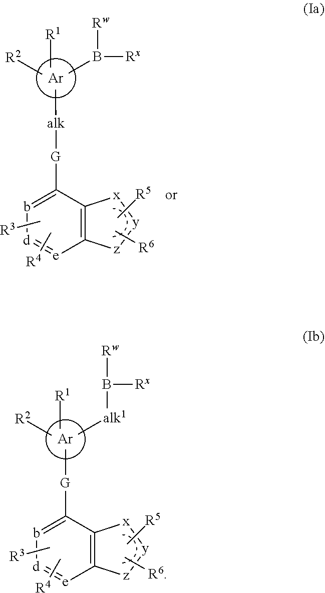

6. The compound of claim 5, or a pharmaceutically acceptable salt thereof, having a structure of formula (Ia).

7. The compound of claim 5, or a pharmaceutically acceptable salt thereof, having a structure of formula (Ib).

8. The compound of any one of claim 3 or 4, or a pharmaceutically acceptable salt thereof, having a structure of formula (Ic) or (Id): 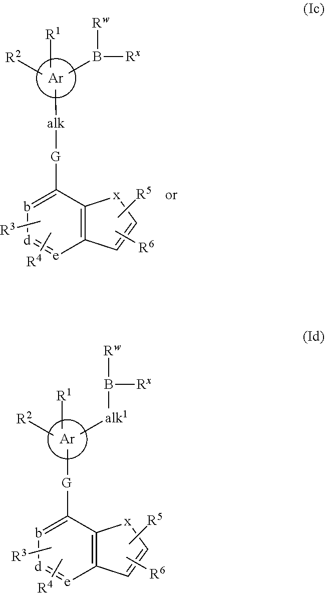
wherein x is NH, O, or S.

9. The compound of claim 8, or a pharmaceutically acceptable salt thereof, having a structure of formula (Ic).

10. The compound of claim 8, or a pharmaceutically acceptable salt thereof, having a structure of formula (Id).

11. The compound of any one of claims 8 to 10, or a pharmaceutically acceptable salt thereof, wherein x is NH.

12. The compound of any one of claims 8 to 10, or a pharmaceutically acceptable salt thereof, wherein x is O.

13. The compound of any one of claims 8 to 10, or a pharmaceutically acceptable salt thereof, wherein x is S.

14. The compound of claim 11, or a pharmaceutically acceptable salt thereof, wherein R⁶ is either attached to the nitrogen of NH or the carbon of the 5-membering ring that is adjacent to x, and R⁵ when present is attached to carbon of the 5-membered ring that is adjacent to a bridgehead carbon.

15. The compound of claim 12 or 13, or a pharmaceutically acceptable salt thereof, wherein R⁶ is attached to the carbon of the 5-membered ring that is adjacent to x and R⁵ when present is attached to carbon of the 5-membered ring that is adjacent to a bridgehead carbon.

16. The compound of any one of claim 3 or 4, or a pharmaceutically acceptable salt thereof, having a structure of formula (Ie) or (If): 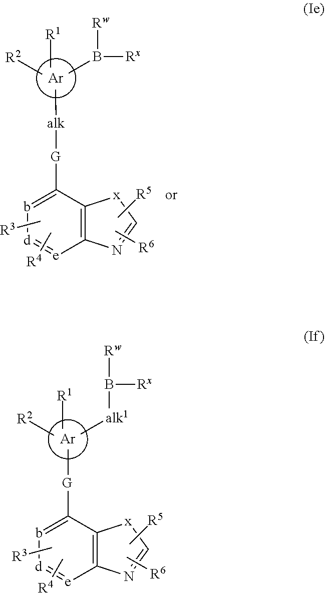
wherein x is NH, O, or S.

17. The compound of claim 16, or a pharmaceutically acceptable salt thereof, having a structure of formula (Ie).

18. The compound of claim 16, or a pharmaceutically acceptable salt thereof, having a structure of formula (If).

19. The compound of any one of claims 16 to 18, or a pharmaceutically acceptable salt thereof, wherein x is NH.

20. The compound of any one of claims 16 to 18, or a pharmaceutically acceptable salt thereof, wherein x is O.

21. The compound of any one of claims 16 to 18, or a pharmaceutically acceptable salt thereof, wherein x is S.

22. The compound of claim 19, or a pharmaceutically acceptable salt thereof, wherein R⁶ is attached to the nitrogen of NH and R⁵ is attached to carbon of the 5-membered ring that is adjacent to x.

23. The compound of claim 20 or 21, or a pharmaceutically acceptable salt thereof, wherein R⁶ is absent and R⁵ is attached to carbon of the 5-membered ring that is adjacent to x.

24. The compound of any one of claim 3 or 4, or a pharmaceutically acceptable salt thereof, having a structure of formula (Ig) or (Ih): 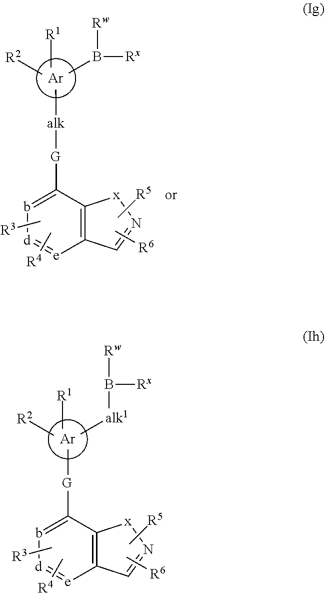
wherein x is NH, O, or S.

25. The compound of claim 24, or a pharmaceutically acceptable salt thereof, having a structure of formula (Ig).

26. The compound of claim 24, or a pharmaceutically acceptable salt thereof, having a structure of formula (Ih).

27. The compound of any one of claims 24 to 26, or a pharmaceutically acceptable salt thereof, wherein x is NH.

28. The compound of any one of claims 24 to 26, or a pharmaceutically acceptable salt thereof, wherein x is O.

29. The compound of any one of claims 24 to 26, or a pharmaceutically acceptable salt thereof, wherein x is S.

30. The compound of any one of claims 27 to 29, or a pharmaceutically acceptable salt thereof, wherein R⁶ is absent and R⁵ is attached to carbon of the 5-membered ring that is adjacent to a bridgehead carbon.

31. The compound of any one of claims 3 to 30, or a pharmaceutically acceptable salt thereof, wherein G is NR.

32. The compound of any one of claims 3 to 30, or a pharmaceutically acceptable salt thereof, wherein G is O.

33. The compound of any one of claims 3 to 32, or a pharmaceutically acceptable salt thereof, wherein b, d, and e are CH.

34. The compound of any one of claims 3 to 32, or a pharmaceutically acceptable salt thereof, wherein b is N and d, and e are CH.

35. The compound of any one of claims 3 to 32, or a pharmaceutically acceptable salt thereof, wherein a and d is N and b and e are CH.

36. The compound of any one of claims 3 to 32, or a pharmaceutically acceptable salt thereof, wherein e is N and b and d are CH.

37. The compound of any one of claims 3 to 32, or a pharmaceutically acceptable salt thereof, wherein b and e are N and d is CH.

38. The compound of any one of claims 3 to 37, or a pharmaceutically acceptable salt thereof, wherein —B(R^{x})(R^{w}) is —B(OH)₂.

39. The compound of any one of claims 3 to 38, or a pharmaceutically acceptable salt thereof, wherein Ar is phenyl.

40. The compound of any one of claims 3 to 38, or a pharmaceutically acceptable salt thereof, wherein Ar is phenyl and —B(R^{w})(R^{x}) is attached to carbon on the phenyl ring that is para to the carbon attaching the phenyl ring to remaining compound of Formula (I), (IA), (IB), and (Ia) to (Ih).

41. The compound of any one of claims 3 to 40, or a pharmaceutically acceptable salt thereof, wherein alk and alk¹ are independently methylene, ethylene, or propylene.

42. The compound of any one of claims 3 to 40, or a pharmaceutically acceptable salt thereof, wherein alk and alk¹ are methylene.

43. The compound of any one of claims 3 to 42, or a pharmaceutically acceptable salt thereof, wherein R⁶ is absent, methyl, or isopropyl.

44. The compound of any one of claims 3 to 43, or a pharmaceutically acceptable salt thereof, wherein R⁵ is absent, alkyl, hydroxy, halo, acylamino, alkoxyalkylamino, cyano, aminocarbonyl, alkylaminocarbonyl, or dialkylaminocarbonyl.

45. The compound of any one of claims 3 to 43, or a pharmaceutically acceptable salt thereof, wherein R⁵ is aminocarbonyl, methylaminocarbonyl, ethylaminocarbonyl, dimethylaminocarbonyl, or isopropylcarbonyl.

46. The compound of any one of claims 3 to 45, or a pharmaceutically acceptable salt thereof, wherein R¹ and R² are independently absent, methyl, ethyl, methoxy, fluoro, trifluoromethyl, trifluoromethoxy, or cyano.

47. The compound of claim 46, or a pharmaceutically acceptable salt thereof, wherein R¹ and R² are absent.

48. The compound of any one of claims 3 to 47, or a pharmaceutically acceptable salt thereof, wherein R³ and R⁴ are independently absent, alkyl, alkoxy, hydroxy, amino, halo, haloalkyl, or haloalkoxy.

49. A pharmaceutical composition comprising a compound any one of claims 1 to 47, and a pharmaceutically acceptable excipient.

50. A method of treating a disease or condition mediated by ENPP1 in a patient comprising administering to the patient a compound of any one of claims 1 to 48, or a pharmaceutical composition of claim 49 in a therapeutically effective amount.

51. The method of claim 50, wherein the disease or condition is a cancer, an inflammatory disease, a metabolic disease, or a viral disease.

52. The method of claim 51, wherein the disease or condition is a cancer.

53. The method of claim 52, wherein the disease or condition is a cancer wherein the cancer is hepatocellular carcinomas, glioblastomas, melanomas, testicular, pancreatic, thyroid or breast cancer.

54. The method of claim 52 or 53, wherein the compound of any one of claims 1 to 47 is administered in combination with another anticancer agent.
